(19) United States
(12) Patent Application Publication
(10) Pub. No.: US 2026/0337616 A1
(43) Pub. Date: Oct. 29, 2026

(54) NOVEL SCN10A RNAi AGENTS AND USES THEREOF

(71) Applicant: ELI LILLY AND COMPANY, Indianapolis, IN (US)

(72) Inventors: Jennifer Rowe CULLEN, Indianapolis, IN (US); MariJean EGGEN, Brownsburg, IN (US); Cheng-Chiu HUANG, Cambridge, MA (US); Theodore Curtis JESSOP, Zionsville, IN (US); Douglas Raymond PERKINS, New Palestine, IN (US); Taisia SHMUSHKOVICH, Cambridge, MA (US); Jibo WANG, Carmel, IN (US)

(21) Appl. No.: 19/484,330

(22) PCT Filed: May 23, 2024
(86) PCT No.: PCT/US2024/030708

§ 371 Date: Nov. 13, 2025

Related U.S. Application Data
(60) Provisional application No. 63/504,654, filed on May 26, 2023.
(60) Provisional application No. 63/571,173, filed on Mar. 28, 2024.

(51) Int. Cl. C12N 15/113; A61K 9/00; A61K 31/712; A61K 31/7125; A61K 31/713

(52) U.S. Cl. CPC C12N 15/1138; A61K 9/0085; A61K 31/712; A61K 31/7125; A61K 31/713; C12N 2310/11; C12N 2310/14; C12N 2310/312; C12N 2310/315; C12N 2310/321; C12N 2310/322; C12N 2310/3515

(57) ABSTRACT

The present invention relates to therapeutic compounds, novel RNA interference (RNAi) agents, that decrease expression of the Voltage-Gated Sodium Channel Alpha Subunit 10 (SCN10A) gene, which encodes a subunit of the Nav 1.8 or Nav 1.8 sodium channel. Such RNAi agents decrease levels of intact Nav 1.8 sodium channels and are useful in the treatment of diseases involving the regulation of SCN10A expression and function, such as chronic pain.

### REFERENCE TO A SEQUENCE LISTING

The present application is being filed along with a Sequence Listing in ST.26 XML The Sequence Listing is provided as a file titled “30493_WO.xml” created May 8, 2024 and is 1.7 megabytes in size. The Sequence Listing information in the ST.26 XML format is incorporated herein by references in its entirety.

The present invention relates to therapeutic compounds, novel RNA interference (RNAi) agents, that decrease expression of the Voltage-Gated Sodium Channel Alpha Subunit 10 (SCN10A) gene, which encodes a subunit of the Nav 1.8 or Nav 1.8 sodium channel. Such RNAi agents decrease levels of intact Nav 1.8 sodium channels and are useful in the treatment of diseases involving the regulation of SCN10A expression and function, such as chronic pain.

Pain stems from neural pathways in response to tissue damaging or potentially tissue damaging signals and may become neuropathic and not occur in response to stimuli. Neuropathic pain tends to relate to dysfunction within the nervous system and is often chronic.

Voltage-gated sodium channels (VGSCs) play an important role in action potential generation and propagation. The rapid influx of sodium ions via VGSCs creates the rising phase of the action potential in sensory nociceptive neurons (Blair and Bean, The Journal of Neuroscience, Dec. 1, 2002, 22 (23): 10277-10290). Nav1.8 channels in particular play a major role in regulating the firing properties of DRG neurons.

Chronic pain is a significant unmet need affecting about 25% of the global population. Current treatments result in tolerance and/or nonselective inhibition of multiple VGSCs.

There remains a need for the treatment of pain, including chronic pain, such as a treatment that exhibits one or more of: improved pain management compared to one or more pain therapies, including a larger decrease in pain levels or a longer duration of pain alleviation; therapy that requires fewer doctor visits or fewer doses as compared to one or more pain therapies, therapy that reduces tolerance; an improved toxicity profile; improved safety profile; fewer side effects; and/or improved tolerability.

### SUMMARY

The RNAi agents disclosed herein for the treatment of pain, including without limitation chronic pain may exhibit improved target knockdown, safety and/or tolerability. The RNAi agents herein may further exhibit additional improved properties, or any combination of two or more of any of the preceding properties.

Accordingly, in some embodiments disclosed herein are SCN10A RNAi agents comprising a sense strand and an antisense strand, wherein the antisense strand comprises a sequence complementary to a sequence selected from any one of SEQ ID NOs: 2-32, wherein the sense strand and the antisense strand form a duplex. In some embodiments, the duplex comprises a sense strand and an antisense strand that are complementary across at least 15, 16, 17, 18, 19, 20 or 21 consecutive nucleotides, or complementary across 15, 16, 17, 18, 19, 20 or 21 consecutive nucleotides. In some embodiments of the duplex, the antisense strand comprises a sequence complementary to at least 15, 16, 17 or 18 consecutive nucleotides from any one of SEQ ID Nos: 2-32. In some embodiments of the duplex, the antisense strand comprises a sequence complementary to 15, 16, 17 or 18 consecutive nucleotides from any one of SEQ ID Nos: 2-32. In some embodiments of the duplex, the sense strand is 21 nucleotides in length and the sense strand comprises a sequence of any one of 33 to 63. In some embodiments of the duplex, the antisense strand is 23 nucleotides in length and the antisense strand comprises a sequence of any one of SEQ ID NOs: 64 to 93.

In some embodiments the RNAi agents comprising a sense strand and an antisense strand, wherein the sense strand and the antisense strand form a duplex, formed by a pair of oligonucleotide sequences selected from the group consisting of:
a. the sense strand has at least 90% sequence identity to SEQ ID NO: 33; and the antisense strand has at least 90% sequence identity to SEQ ID NO:64;
b. the sense strand has at least 90% sequence identity to SEQ ID NO: 34; and the antisense strand has at least 90% sequence identity to SEQ ID NO:65;
c. the sense strand has at least 90% sequence identity to SEQ ID NO: 35; and the antisense strand has at least 90% sequence identity to SEQ ID NO:66;
d. the sense strand has at least 90% sequence identity to SEQ ID NO: 36; and the antisense strand has at least 90% sequence identity to SEQ ID NO:67;
e. the sense strand has at least 90% sequence identity to SEQ ID NO: 37; and the antisense strand has at least 90% sequence identity to SEQ ID NO:68;
f. the sense strand has at least 90% sequence identity to SEQ ID NO: 38; and the antisense strand has at least 90% sequence identity to SEQ ID NO:69;
g. the sense strand has at least 90% sequence identity to SEQ ID NO: 39; and the antisense strand has at least 90% sequence identity to SEQ ID NO:70;
h. the sense strand has at least 90% sequence identity to SEQ ID NO: 40; and the antisense strand has at least 90% sequence identity to SEQ ID NO:71;
i. the sense strand has at least 90% sequence identity to SEQ ID NO: 41; and the antisense strand has at least 90% sequence identity to SEQ ID NO:72;
j. the sense strand has at least 90% sequence identity to SEQ ID NO: 42; and the antisense strand has at least 90% sequence identity to SEQ ID NO:73;
k. the sense strand has at least 90% sequence identity to SEQ ID NO: 43; and the antisense strand has at least 90% sequence identity to SEQ ID NO:74;
l. the sense strand has at least 90% sequence identity to SEQ ID NO: 44; and the antisense strand has at least 90% sequence identity to SEQ ID NO:75;
m. the sense strand has at least 90% sequence identity to SEQ ID NO: 45; and the antisense strand has at least 90% sequence identity to SEQ ID NO:76;
n. the sense strand has at least 90% sequence identity to SEQ ID NO: 46; and the antisense strand has at least 90% sequence identity to SEQ ID NO:77;
0, the sense strand has at least 90% sequence identity to SEQ ID NO: 47; and the antisense strand has at least 90% sequence identity to SEQ ID NO:78;
p. the sense strand has at least 90% sequence identity to SEQ ID NO: 48; and the antisense strand has at least 90% sequence identity to SEQ ID NO:79;
q. the sense strand has at least 90% sequence identity to SEQ ID NO: 49; and the antisense strand has at least 90% sequence identity to SEQ ID NO:80;
r. the sense strand has at least 90% sequence identity to SEQ ID NO: 50; and the antisense strand has at least 90% sequence identity to SEQ ID NO:81;
s. the sense strand has at least 90% sequence identity to SEQ ID NO: 51; and the antisense strand has at least 90% sequence identity to SEQ ID NO:82;
t. the sense strand has at least 90% sequence identity to SEQ ID NO: 52; and the antisense strand has at least 90% sequence identity to SEQ ID NO:83;
u. the sense strand has at least 90% sequence identity to SEQ ID NO: 53; and the antisense strand has at least 90% sequence identity to SEQ ID NO:84;
v. the sense strand has at least 90% sequence identity to SEQ ID NO: 54; and the antisense strand has at least 90% sequence identity to SEQ ID NO:85;
w. the sense strand has at least 90% sequence identity to SEQ ID NO: 55; and the antisense strand has at least 90% sequence identity to SEQ ID NO:86;
x. the sense strand has at least 90% sequence identity to SEQ ID NO: 56; and the antisense strand has at least 90% sequence identity to SEQ ID NO:87;
y. the sense strand has at least 90% sequence identity to SEQ ID NO: 57; and the antisense strand has at least 90% sequence identity to SEQ ID NO:88;
z. the sense strand has at least 90% sequence identity to SEQ ID NO: 58; and the antisense strand has at least 90% sequence identity to SEQ ID NO:89;
aa. the sense strand has at least 90% sequence identity to SEQ ID NO: 59; and the antisense strand has at least 90% sequence identity to SEQ ID NO:90;
bb. the sense strand has at least 90% sequence identity to SEQ ID NO: 60; and the antisense strand has at least 90% sequence identity to SEQ ID NO:91;
cc. the sense strand has at least 90% sequence identity to SEQ ID NO: 61; and the antisense strand has at least 90% sequence identity to SEQ ID NO:92;
dd. the sense strand has at least 90% sequence identity to SEQ ID NO: 62; and the antisense strand has at least 90% sequence identity to SEQ ID NO:93; or
ee. the sense strand has at least 90% sequence identity to SEQ ID NO: 63; and the antisense strand has at least 90% sequence identity to SEQ ID NO:66.

In some embodiments the RNAi agents comprise a pair of a sense strand and an antisense strand as described in Table 2, Table 3A and Table 3B.

In some embodiments, the RNAi agents comprise a delivery moiety. In some embodiments, the delivery moiety is a lipophilic delivery moiety. In some embodiments, the delivery moiety is conjugated to a nucleotide at position 12 or 13 from the 5′end of the sense strand. In some embodiments, the lipophilic delivery moiety is optionally of Formula I or 2′-O-hexadecyl. In some embodiments, the sense and antisense strand each independently comprise one or more modified nucleotides, and optionally one or more internucleotide linkages of the sense strand or the antisense strand are modified internucleotide linkages. In some embodiments, the one or more modified nucleotides are independently a 2′-fluoro modified nucleotide or a 2′-O-methyl modified nucleotide. In some embodiments, each nucleotide of the sense and each nucleotide of the antisense strand is modified.

Additional embodiments provide the use of RNAi agents of the invention in pain therapy, including where the therapy is for chronic pain. Embodiments also provide methods of treating pain, including without limitation chronic pain comprising administering a therapeutic dose of an RNAi agent of the invention or a pharmaceutical composition thereof to a subject.

### DETAILED DESCRIPTION

The RNAi agents herein comprise a sense strand and an antisense strand, wherein each is an oligonucleotide. As used herein, “nucleotide” means an organic compound having a nucleoside (a nucleobase such as, for example, adenine, cytosine, guanine, thymine, or uracil; and a pentose sugar such as, for example, ribose or 2′-deoxyribose) and a phosphate group. A “nucleotide” can be a “modified nucleotide.” A “nucleotide” can serve as a monomeric unit of nucleic acid polymers such as deoxyribonucleic acid (DNA) and ribonucleic acid (RNA).

As used herein, an “oligonucleotide” means a short nucleic acid compound (e.g., less than about 100 nucleotides in length). An oligonucleotide may be single-stranded (ss) or double stranded (ds). An oligonucleotide may or may not have duplex regions. As a set of non-limiting examples, an oligonucleotide may be, but is not limited to, a small interfering RNA (siRNA), microRNA (miRNA), short hairpin RNA (shRNA), Dicer substrate interfering RNA (DsiRNA), or antisense oligonucleotide (ASO).

As used herein, “ribonucleotide” means a nucleotide having a ribose as its pentose sugar, which contains a hydroxyl group at its 2′ position. A modified ribonucleotide is a ribonucleotide having one or more modifications or substitutions of atoms other than hydrogen at the 2′ position, including modifications or substitutions in or of the nucleobase, sugar, or phosphate group.

As used herein, a “modified internucleotide linkage” means an internucleotide linkage having one or more chemical modifications when compared with a reference internucleotide linkage having a phosphodiester bond. A modified internucleotide linkage can be a non-naturally occurring linkage.

As used herein, a “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can be a non-naturally occurring nucleotide. A modified nucleotide can have, for example, one or more chemical modification in its sugar, nucleobase, and/or phosphate group, or any combination thereof. Additionally, or alternatively, a modified nucleotide can have one or more chemical moieties conjugated to a corresponding reference nucleotide.

As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate. A 5′ phosphate analog can include a phosphatase-resistant linkage. Examples of phosphate analogs include, but are not limited to, 5′ phosphonates, such as 5′ methylene phosphonate (5′-MP) and 5′-(E)-vinylphosphonate (5′-VP). An oligonucleotide can have a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e g., Intl. Patent Application Publication No. WO 2018/045317.

Other modifications have been developed for the 5′ end of oligonucleotides (see, e.g., Intl. Patent Application No. WO 2011/133871; U.S. Pat. No. 8,927,513; and Prakash et al. (2015) Nuc. Acids Res. 43:2993-3011).

The term “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN or Megalign (DNASTAR), Clustal W2.0 or Clustal X2.0 software. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence. In some embodiments, percent sequence identity is the percent of nucleotide residues that are identical between two strands using the PID3 calculation, which is the number of identical nucleotide residues divided by the total number of nucleotides of the shortest of the two sequences, multiplied by 100. See, e.g., Raghava, G., Barton, G. J. Quantification of the variation in percentage identity for protein sequence alignments. BMC Bioinformatics 7, 415 (2006). Herein, in one embodiment, the alignment is done using Clustal W2.0. In another embodiment, the alignment is done using Clustal X2.0. In either of the preceding embodiments, following the Clustal alignment, the percentage of nucleotide residues that are identical between two strands is done using the PID3 calculation.

As used herein, “complementary” or “complementarity” means a structural relationship between two nucleotides, nucleosides, or nucleobases (e.g., on two opposing nucleic acids or on opposing regions of a single nucleic acid strand e.g., a hairpin) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the canonical Watson-Crick manner, which means adenine pairing with thymine or uracil, and guanine pairing with cytosine, or in any other manner that allows for the formation of stable duplexes. Likewise, two nucleic acids may have regions of multiple nucleotides that are complementary with each other to form regions of complementarity. As used herein, “region of complementarity” means a nucleotide sequence of a nucleic acid that is complementary to an antiparallel nucleotide sequence to permit hybridization between the two sequences of nucleotides under appropriate hybridization conditions (e.g., in a phosphate buffer, in a cell, etc.). In some embodiments, an antisense oligonucleotide includes a region of complementarity to a mRNA target sequence.

As used herein, a “duplex,” means a structure formed through hydrogen bonds of complementary base pairing of two antiparallel sequences of nucleotides under suitable conditions to promote such a structure. A duplex may form despite not having full complementarity between the two strands, or when an abasic nucleotide is present.

As used herein, “iRNA,” “iRNA agent,” “RNAi,” “RNAi agent,” “siRNA” and “RNA interference agent” means an agent that contains RNA and mediates the targeted cleavage of an RNA transcript via RNA interference, e.g., through a RNA-induced silencing complex (RISC) pathway. In some embodiments, the sense and antisense strands of RNAi agent are 21-23 nucleotides in length. In other embodiments, the sense and antisense strands can be longer, for example 25-30 nucleotides in length, in which case the longer RNAi sequences are first processed by the Dicer enzyme. The iRNA attenuates, inhibits, modulates, or reduces the target gene expression in a cell.

As used herein, “conjugated to”, “conjugate”, or “conjugated” means a covalent bond, whether indirect or direct. For example, the sense or antisense strands disclosed herein may be covalently bonded to a delivery moiety to facilitate entry into the cells of CNS tissues. In some embodiments, the sense or antisense strand is covalently bonded directly to the delivery moiety. In other embodiments, the sense or the antisense strand is conjugated indirectly via a linker; that is the sense strand or the antisense strand is covalently bonded to an atom of the linker, and the linker is in turn covalently bonded to the delivery moiety.

As used herein, “linker” means a structure used to conjugate a nucleotide (e.g., oligonucleotide) to a targeting ligand or a delivery moiety. In some embodiments, a linker can be “labile” or “cleavable” meaning a linker that can be cleaved (e.g., by acidic pH or enzyme). In other embodiments, a linker can be “stable” or “non-cleavable” meaning a linker that is not cleaved in physiological conditions.

As used herein, “reduced expression,” “reduction of expression,” or “reducing expression”, such as with respect to a gene, means a decrease in the amount or level of RNA transcript and/or protein encoded by the gene and/or a decrease in the level of activity of the gene in a cell, a population of cells, a sample, or a subject, when compared to an appropriate reference (e.g., a reference cell, population of cells, sample, or subject). For example, introducing an RNAi agent herein (e.g., an oligonucleotide having an antisense strand having a nucleotide sequence that is complementary to SCN10A mRNA or a portion thereof) into a cell may result in a decrease in the amount or level of mRNA, protein, and/or activity when compared to a cell that is not treated with the RNAi agent. Specifically, and as used herein, “reduction of SCN10A expression” means a decrease in the amount or level of SCN10A mRNA, SCN10A protein level, and/or SCN10A protein activity in a cell, a population of cells, a sample, or a subject when compared to an appropriate reference that is not treated with RNAi agent of the invention (e.g., a reference cell, population of cells, a tissue, or a subject).

As used herein, “patient” means any mammal, including cats, dogs, mice, rats, and primates, and humans that express or are engineered to express a human Nav 1.8 gene. Moreover, “patient” and “subject” may be used interchangeably.

As used herein, “treatment” or “treating” refers to all processes wherein there may be a slowing, controlling, delaying, or stopping of the progression of the disorders or disease disclosed herein, or ameliorating disorder or disease symptoms, and need not indicate a total elimination of all disorder or disease symptoms. Treatment includes administration of an RNAi agent or pharmaceutical composition thereof for treatment of a disease or condition in a mammal including a human.

Disclosed herein are RNAi agents having a sense strand and an antisense strand that are engineered to decrease the expression of the SCN10A gene. One embodiment is an RNAi agent having a sense strand and an antisense strand, wherein the antisense strand is complementary to the mRNA transcript of the SCN10A gene, and wherein the antisense strand and the sense strand form a duplex.

In certain embodiments, the RNAi agent is capable of decreasing expression of the SCN10A gene in a cell. In other embodiments, the RNAi agent is capable of decreasing expression of the SCN10A gene in human DRG cells. In other embodiments, the RNAi agent is capable of decreasing expression of the SCN10A gene in vivo.

As described above, the RNAi agents can be modified. Accordingly, in further embodiments, the antisense strand or the sense strand optionally comprise one or more modified nucleotides, and optionally one or more internucleotide linkages of the sense strand or the antisense strand are modified internucleotide linkages.

In one embodiment an RNAi agent comprises a sense strand and an antisense strand, wherein the antisense strand comprises a sequence complementary to a sequence selected from any one of SEQ ID NOs: 2-32, wherein the sense strand and the antisense strand form a duplex, and optionally wherein the sense and antisense strand each independently comprise one or more modified nucleotides, and optionally wherein one or more internucleotide linkages of the sense strand or the antisense strand are modified internucleotide linkages.

In a further embodiment, the antisense strand comprises a 3′ overhang of two nucleotides. In a further embodiment, the antisense strand and the sense strand are independently 15 to 50 nucleotides. In a further embodiment, the antisense strand and the sense strand are independently 18 to 23 nucleotides. In a further embodiment, the antisense strand is 23 nucleotides in length. In a further embodiment, the sense strand is 21 nucleotides in length. In a further embodiment, the antisense strand is 23 nucleotides in length and the sense strand is 21 nucleotides in length.

In some embodiments, an RNAi comprises a sense strand and an antisense strand that form a duplex, the antisense strand is 23 nucleotides in length and the sense strand is 21 nucleotides in length. In some embodiments of the duplex, the antisense strand comprises a sequence that is complementary to at least 15, 16, 17 or 18 consecutive nucleotides of a sequence selected from any one of SEQ ID NOs: 2-32. In some embodiments, the duplex comprises a sense strand and an antisense strand that are complementary across at least 15, 16, 17, 18, 19, 20 or 21 consecutive nucleotides, or complementary across 15, 16, 17, 18, 19, 20 or 21 consecutive nucleotides. In further embodiments, the antisense strand comprises a sequence of any one of SEQ ID NOS: 64 to 93. In a further embodiment, the sense strand comprises a sequence of any one of SEQ ID NOs: 33 to 63. In some embodiments, the RNAi agent comprises a duplex comprising the sense strand and the antisense strand pair listed in Table 2, Table 3A or Table 3B. In some embodiments of the duplex, the sense strand comprises SEQ ID NO: 35 and the antisense strand comprises SEQ ID NO:66. In some embodiments, the sense strand comprises SEQ ID NO: 39 and the antisense strand comprises SEQ ID NO: 70.

In some embodiments of the RNAi agents, the sense strand and antisense strand form a duplex, and wherein:
a. the sense strand has at least 90% sequence identity to SEQ ID NO: 33; and the antisense strand has at least 90% sequence identity to SEQ ID NO:64;
b. the sense strand has at least 90% sequence identity to SEQ ID NO: 34; and the antisense strand has at least 90% sequence identity to SEQ ID NO:65;
c. the sense strand has at least 90% sequence identity to SEQ ID NO: 35; and the antisense strand has at least 90% sequence identity to SEQ ID NO:66;
d. the sense strand has at least 90% sequence identity to SEQ ID NO: 36; and the antisense strand has at least 90% sequence identity to SEQ ID NO:67;
e. the sense strand has at least 90% sequence identity to SEQ ID NO: 37; and the antisense strand has at least 90% sequence identity to SEQ ID NO:68;
f. the sense strand has at least 90% sequence identity to SEQ ID NO: 38; and the antisense strand has at least 90% sequence identity to SEQ ID NO:69;
g. the sense strand has at least 90% sequence identity to SEQ ID NO: 39; and the antisense strand has at least 90% sequence identity to SEQ ID NO:70;
h. the sense strand has at least 90% sequence identity to SEQ ID NO: 40; and the antisense strand has at least 90% sequence identity to SEQ ID NO:71;
i. the sense strand has at least 90% sequence identity to SEQ ID NO: 41; and the antisense strand has at least 90% sequence identity to SEQ ID NO: 72;
j. the sense strand has at least 90% sequence identity to SEQ ID NO: 42; and the antisense strand has at least 90% sequence identity to SEQ ID NO:73;
k. the sense strand has at least 90% sequence identity to SEQ ID NO: 43; and the antisense strand has at least 90% sequence identity to SEQ ID NO:74;
l. the sense strand has at least 90% sequence identity to SEQ ID NO: 44; and the antisense strand has at least 90% sequence identity to SEQ ID NO:75;
m. the sense strand has at least 90% sequence identity to SEQ ID NO: 45; and the antisense strand has at least 90% sequence identity to SEQ ID NO:76;
n. the sense strand has at least 90% sequence identity to SEQ ID NO: 46; and the antisense strand has at least 90% sequence identity to SEQ ID NO:77;
o, the sense strand has at least 90% sequence identity to SEQ ID NO: 47; and the antisense strand has at least 90% sequence identity to SEQ ID NO:78;
p. the sense strand has at least 90% sequence identity to SEQ ID NO: 48; and the antisense strand has at least 90% sequence identity to SEQ ID NO:79;
q. the sense strand has at least 90% sequence identity to SEQ ID NO: 49; and the antisense strand has at least 90% sequence identity to SEQ ID NO:80;
r. the sense strand has at least 90% sequence identity to SEQ ID NO: 50; and the antisense strand has at least 90% sequence identity to SEQ ID NO:81;
s. the sense strand has at least 90% sequence identity to SEQ ID NO: 51; and the antisense strand has at least 90% sequence identity to SEQ ID NO:82;
t. the sense strand has at least 90% sequence identity to SEQ ID NO: 52; and the antisense strand has at least 90% sequence identity to SEQ ID NO:83;
u. the sense strand has at least 90% sequence identity to SEQ ID NO: 53; and the antisense strand has at least 90% sequence identity to SEQ ID NO:84;
v. the sense strand has at least 90% sequence identity to SEQ ID NO: 54; and the antisense strand has at least 90% sequence identity to SEQ ID NO:85;
w. the sense strand has at least 90% sequence identity to SEQ ID NO: 55; and the antisense strand has at least 90% sequence identity to SEQ ID NO:86;
x. the sense strand has at least 90% sequence identity to SEQ ID NO: 56; and the antisense strand has at least 90% sequence identity to SEQ ID NO:87;
y. the sense strand has at least 90% sequence identity to SEQ ID NO: 57; and the antisense strand has at least 90% sequence identity to SEQ ID NO:88;
z. the sense strand has at least 90% sequence identity to SEQ ID NO: 58; and the antisense strand has at least 90% sequence identity to SEQ ID NO:89;
aa. the sense strand has at least 90% sequence identity to SEQ ID NO: 59; and the antisense strand has at least 90% sequence identity to SEQ ID NO:90;
bb. the sense strand has at least 90% sequence identity to SEQ ID NO: 60; and the antisense strand has at least 90% sequence identity to SEQ ID NO:91;
cc. the sense strand has at least 90% sequence identity to SEQ ID NO: 61; and the antisense strand has at least 90% sequence identity to SEQ ID NO:92;
dd. the sense strand has at least 90% sequence identity to SEQ ID NO: 62; and the antisense strand has at least 90% sequence identity to SEQ ID NO:93; or
ee. the sense strand has at least 90% sequence identity to SEQ ID NO: 63; and the antisense strand has at least 90% sequence identity to SEQ ID NO:66.

In some embodiments of the RNAi agents, the duplex comprises a sense strand and an antisense strand wherein:
a. the sense strand comprises SEQ ID NO:94 and the antisense strand comprises SEQ ID NO:132;
b. the sense strand comprises SEQ ID NO:95 and the antisense strand comprises SEQ ID NO:133;
c. the sense strand comprises SEQ ID NO:96 and the antisense strand comprises SEQ ID NO:134;
d. the sense strand comprises SEQ ID NO:97 and the antisense strand comprises SEQ ID NO:135;
e. the sense strand comprises SEQ ID NO:98 and the antisense strand comprises SEQ ID NO:136;
f. the sense strand comprises SEQ ID NO:99 and the antisense strand comprises SEQ ID NO:137;
g. the sense strand comprises SEQ ID NO: 100 and the antisense strand comprises SEQ ID NO:138;
h. the sense strand comprises SEQ ID NO:101 the antisense strand comprises SEQ ID NO:139;
i. the sense strand comprises SEQ ID NO: 102 and the antisense strand comprises SEQ ID NO:140;
j. the sense strand comprises SEQ ID NO: 103 and the antisense strand comprises SEQ ID NO:141;
k. the sense strand comprises SEQ ID NO: 104 and the antisense strand comprises SEQ ID NO:142;
l. the sense strand comprises SEQ ID NO: 105 and the antisense strand comprises SEQ ID NO:143;
m. the sense strand comprises SEQ ID NO: 106 and the antisense strand comprises SEQ ID NO:144;
n. the sense strand comprises SEQ ID NO: 107 and the antisense strand comprises SEQ ID NO:145;
o. the sense strand comprises SEQ ID NO: 108 and the antisense strand comprises SEQ ID NO:146;
p. the sense strand comprises SEQ ID NO: 109 and the antisense strand comprises SEQ ID NO:147;
q. the sense strand comprises SEQ ID NO:110 and the antisense strand comprises SEQ ID NO:148;
r. the sense strand comprises SEQ ID NO:111 and the antisense strand comprises SEQ ID NO:149;
s. the sense strand comprises SEQ ID NO: 112 and the antisense strand comprises SEQ ID NO:150;
t. the sense strand comprises SEQ ID NO: 113 and the antisense strand comprises SEQ ID NO:151;
u. the sense strand comprises SEQ ID NO: 114 and the antisense strand comprises SEQ ID NO:152;
v. the sense strand comprises SEQ ID NO: 115 and the antisense strand comprises SEQ ID NO:153;
w. the sense strand comprises SEQ ID NO: 116 and the antisense strand comprises SEQ ID NO:154;
x. the sense strand comprises SEQ ID NO: 117 and the antisense strand comprises SEQ ID NO:155;
y. the sense strand comprises SEQ ID NO: 118 and the antisense strand comprises SEQ ID NO:156;
z. the sense strand comprises SEQ ID NO:119 and the antisense strand comprises SEQ ID NO:157;
aa. the sense strand comprises SEQ ID NO: 120 and the antisense strand comprises SEQ ID NO:158;
bb. the sense strand comprises SEQ ID NO:121 and the antisense strand comprises SEQ ID NO:159;
cc. the sense strand comprises SEQ ID NO: 122 and the antisense strand comprises SEQ ID NO:160;
dd. the sense strand comprises SEQ ID NO:123 and the antisense strand comprises SEQ ID NO:161;
ee. the sense strand comprises SEQ ID NO:96 and the antisense strand comprises SEQ ID NO:162;
ff. the sense strand comprises SEQ ID NO:96 and the antisense strand comprises SEQ ID NO:163;
gg. the sense strand comprises SEQ ID NO:96 and the antisense strand comprises SEQ ID NO:164;
hh. the sense strand comprises SEQ ID NO:96 and the antisense strand comprises SEQ ID NO:165;
ii. the sense strand comprises SEQ ID NO:96 and the antisense strand comprises SEQ ID NO:166;
jj. the sense strand comprises SEQ ID NO: 124 and the antisense strand comprises SEQ ID NO:162;
kk. the sense strand comprises SEQ ID NO: 124 and the antisense strand comprises SEQ ID NO:163;
ll. the sense strand comprises SEQ ID NO:124 and the antisense strand comprises SEQ ID NO:164;
mm. the sense strand comprises SEQ ID NO:12; and the antisense strand comprises SEQ ID NO:165;
nn. the sense strand comprises SEQ ID NO:124 and the antisense strand comprises SEQ ID NO:166;
oo. the sense strand comprises SEQ ID NO: 124 and the antisense strand comprises SEQ ID NO:134;
pp. the sense strand comprises SEQ ID NO: 125 and the antisense strand comprises SEQ ID NO:162;
qq. the sense strand comprises SEQ ID NO: 125 and the antisense strand comprises SEQ ID NO:163;
rr. the sense strand comprises SEQ ID NO:125 and the antisense strand comprises SEQ ID NO:164;
ss. the sense strand comprises SEQ ID NO: 125 and the antisense strand comprises SEQ ID NO:165;
tt. the sense strand comprises SEQ ID NO: 125 and the antisense strand comprises SEQ ID NO:166;
uu. the sense strand comprises SEQ ID NO:125 and the antisense strand comprises SEQ ID NO: 134;
vv. the sense strand comprises SEQ ID NO: 126 and the antisense strand comprises SEQ ID NO: 162;
ww. the sense strand comprises SEQ ID NO: 126 and the antisense strand comprises SEQ ID NO:163;
xx. the sense strand comprises SEQ ID NO: 126 and the antisense strand comprises SEQ ID NO:164;
yy. the sense strand comprises SEQ ID NO:126 and the antisense strand comprises SEQ ID NO:165;
zz. the sense strand comprises SEQ ID NO:126 and the antisense strand comprises SEQ ID NO:166;
aaa. the sense strand comprises SEQ ID NO: 126 and the antisense strand comprises SEQ ID NO: 134;
bbb. the sense strand comprises SEQ ID NO: 127 and the antisense strand comprises SEQ ID NO:167;
ccc. the sense strand comprises SEQ ID NO: 127 and the antisense strand comprises SEQ ID NO:168;
ddd. the sense strand comprises SEQ ID NO: 127 and the antisense strand comprises SEQ ID NO:169;
eee. the sense strand comprises SEQ ID NO: 128 and the antisense strand comprises SEQ ID NO:170;
fff. the sense strand comprises SEQ ID NO: 128 and the antisense strand comprises SEQ ID NO:171;
ggg. the sense strand comprises SEQ ID NO:128 and the antisense strand comprises SEQ ID NO:172;
hhh. the sense strand comprises SEQ ID NO: 129 and the antisense strand comprises SEQ ID NO:173;
iii. the sense strand comprises SEQ ID NO:129 and the antisense strand comprises SEQ ID NO:174;
jjj. the sense strand comprises SEQ ID NO: 129 and the antisense strand comprises SEQ ID NO:175;
kkk. the sense strand comprises SEQ ID NO:130 and the antisense strand comprises SEQ ID NO:176;
lll. the sense strand comprises SEQ ID NO: 130 and the antisense strand comprises SEQ ID NO:177;
mmm. the sense strand comprises SEQ ID NO: 130 and the antisense strand comprises SEQ ID NO: 178;
nnn. the sense strand comprises SEQ ID NO:131 and the antisense strand comprises SEQ ID NO:179;
ooo. the sense strand comprises SEQ ID NO: 131 and the antisense strand comprises SEQ ID NO:180;
ppp. the sense strand comprises SEQ ID NO: 131 and the antisense strand comprises SEQ ID NO:181;
qqq. the sense strand comprises SEQ ID NO:232 and the antisense strand comprises SEQ ID NO:233;
rrr. the sense strand comprises SEQ ID NO:234 and the antisense strand comprises SEQ ID NO:235;
sss. the sense strand comprises SEQ ID NO:236 and the antisense strand comprises SEQ ID NO:233;
ttt. the sense strand comprises SEQ ID NO:237 and the antisense strand comprises SEQ ID NO:233;
uuu. the sense strand comprises to SEQ ID NO:238 and the antisense strand comprises SEQ ID NO:239;
vvv. the sense strand comprises SEQ ID NO:240 and the antisense strand comprises SEQ ID NO:239;
www. the sense strand comprises SEQ ID NO:238 and the antisense strand comprises SEQ ID NO:235;
xxx. the sense strand comprises SEQ ID NO:240 and the antisense strand comprises SEQ ID NO:235;
yyy. the sense strand comprises SEQ ID NO:241 and the antisense strand comprises SEQ ID NO:235;
zzz. the sense strand comprises SEQ ID NO:238 and the antisense strand comprises SEQ ID NO:242;
aaaa. the sense strand comprises SEQ ID NO:232 and the antisense strand comprises SEQ ID NO:239;
bbbb. the sense strand comprises SEQ ID NO:240 and the antisense strand comprises SEQ ID NO:242;
cccc. the sense strand comprises SEQ ID NO:94 and the antisense strand comprises SEQ ID NO:182;
dddd. the sense strand comprises SEQ ID NO:95 and the antisense strand comprises SEQ ID NO: 183;
eeee. the sense strand comprises SEQ ID NO:96 and the antisense strand comprises SEQ ID NO: 184;
ffff. the sense strand comprises SEQ ID NO:97 and the antisense strand comprises SEQ ID NO:185;
gggg. the sense strand comprises SEQ ID NO:98 and the antisense strand comprises SEQ ID NO:186;
hhhh. the sense strand comprises SEQ ID NO:99 and the antisense strand comprises SEQ ID NO:187;
iiii. the sense strand comprises SEQ ID NO:100 and the antisense strand comprises SEQ ID NO:188;
jjjj. the sense strand comprises SEQ ID NO:101 and the antisense strand comprises SEQ ID NO:189;
kkkk. the sense strand comprises SEQ ID NO: 102 and the antisense strand comprises SEQ ID NO:190;
llll. the sense strand comprises SEQ ID NO: 103 and the antisense strand comprises SEQ ID NO:191;
mmmm. the sense strand comprises SEQ ID NO: 104 and the antisense strand comprises SEQ ID NO:192;
nnnn. the sense strand comprises SEQ ID NO: 105 and the antisense strand comprises SEQ ID NO:193;
oooo. the sense strand comprises SEQ ID NO:106 and the antisense strand comprises SEQ ID NO:194;
pppp. the sense strand comprises SEQ ID NO:107 and the antisense strand comprises SEQ ID NO:195;
qqqq. the sense strand comprises SEQ ID NO:108 and the antisense strand comprises SEQ ID NO:196;
rrrr. the sense strand comprises SEQ ID NO:109 and the antisense strand comprises SEQ ID NO:197;
ssss. the sense strand comprises SEQ ID NO:110 and the antisense strand comprises SEQ ID NO:198;
tttt. the sense strand comprises SEQ ID NO:111 and the antisense strand comprises SEQ ID NO: 199;
uuuu. the sense strand comprises SEQ ID NO:112 and the antisense strand comprises SEQ ID NO:200;
vvvv. the sense strand comprises o SEQ ID NO:113 and the antisense strand comprises SEQ ID NO:201;
wwww. the sense strand comprises SEQ ID NO: 114 and the antisense strand comprises SEQ ID NO:202;
xxxx. the sense strand comprises SEQ ID NO: 115 and the antisense strand comprises SEQ ID NO:203;
yyyy, the sense strand comprises SEQ ID NO:116 and the antisense strand comprises SEQ ID NO:204;
zzzz. the sense strand comprises SEQ ID NO: 117 and the antisense strand comprises SEQ ID NO:205;
aaaaa. the sense strand comprises SEQ ID NO:118 and the antisense strand comprises SEQ ID NO:206;
bbbbb. the sense strand comprises SEQ ID NO:119 and the antisense strand comprises SEQ ID NO:207;
ccccc. the sense strand comprises SEQ ID NO: 120 and the antisense strand comprises SEQ ID NO:208;
ddddd. the sense strand comprises SEQ ID NO: 121 and the antisense strand comprises SEQ ID NO:209;
eeeee. the sense strand comprises SEQ ID NO: 122 and the antisense strand comprises SEQ ID NO:210;
fffff. the sense strand comprises SEQ ID NO: 123 and the antisense strand comprises SEQ ID NO:211;
ggggg. the sense strand comprises SEQ ID NO:96 and the antisense strand comprises SEQ ID NO:212;
hhhhh. the sense strand comprises SEQ ID NO:96 and the antisense strand comprises SEQ ID NO:213;
iiiii. the sense strand comprises SEQ ID NO:96 and the antisense strand comprises SEQ ID NO:214;
jjjjj. the sense strand comprises SEQ ID NO:96 and the antisense strand comprises SEQ ID NO:215;
kkkkk. the sense strand comprises SEQ ID NO:96 and the antisense strand comprises SEQ ID NO:216;
lllll. the sense strand comprises SEQ ID NO:124 and the antisense strand comprises SEQ ID NO:212;
mmmmm. the sense strand comprises SEQ ID NO: 124 and the antisense strand comprises SEQ ID NO:213;
nnnnn. the sense strand comprises SEQ ID NO: 124 and the antisense strand comprises SEQ ID NO:214;
ooooo. the sense strand comprises SEQ ID NO:124 and the antisense strand comprises SEQ ID NO:215;
ppppp. the sense strand comprises SEQ ID NO: 124 and the antisense strand comprises SEQ ID NO:216;
qqqqq. the sense strand comprises SEQ ID NO: 124 and the antisense strand comprises SEQ ID NO:184;
rrrrr. the sense strand comprises SEQ ID NO: 125 and the antisense strand comprises SEQ ID NO:212;
sssss. the sense strand comprises SEQ ID NO: 125 and the antisense strand comprises SEQ ID NO:213;
ttttt, the sense strand comprises SEQ ID NO:125 and the antisense strand comprises SEQ ID NO:214;
uuuuu, the sense strand comprises SEQ ID NO: 125 and the antisense strand comprises SEQ ID NO:215
vvvvv. the sense strand comprises SEQ ID NO: 125 and the antisense strand comprises SEQ ID NO:216;
wwwww. the sense strand comprises SEQ ID NO: 125 and the antisense strand comprises SEQ ID NO:184;
xxxxx. the sense strand comprises SEQ ID NO: 126 and the antisense strand comprises SEQ ID NO:212;
yyyyy. the sense strand comprises SEQ ID NO:126 and the antisense strand comprises SEQ ID NO:213;
zzzzz. the sense strand comprises SEQ ID NO:126 and the antisense strand comprises SEQ ID NO:214;
aaaaaa. the sense strand comprises SEQ ID NO:126 and the antisense strand comprises SEQ ID NO:215;
bbbbbb. the sense strand comprises SEQ ID NO:126 and the antisense strand comprises SEQ ID NO:216;
cccccc. the sense strand comprises SEQ ID NO: 126 and the antisense strand comprises SEQ ID NO:184;
dddddd. the sense strand comprises SEQ ID NO: 127 and the antisense strand comprises SEQ ID NO:217;
eeeeee. the sense strand comprises SEQ ID NO: 127 and the antisense strand comprises SEQ ID NO:218;
ffffff. the sense strand comprises SEQ ID NO: 127 and the antisense strand comprises SEQ ID NO:219;
gggggg. the sense strand comprises SEQ ID NO: 128 and the antisense strand comprises SEQ ID NO:220;
hhhhhh. the sense strand comprises SEQ ID NO: 128 and the antisense strand comprises SEQ ID NO:221;
iiiiii. the sense strand comprises SEQ ID NO: 128 and the antisense strand comprises SEQ ID NO:222;
jjjjjj. the sense strand comprises SEQ ID NO: 129 and the antisense strand comprises SEQ ID NO:223;
kkkkkk. the sense strand comprises SEQ ID NO:129 and the antisense strand comprises SEQ ID NO:224;
llllll. the sense strand comprises SEQ ID NO: 129 and the antisense strand comprises SEQ ID NO:225;
mmmmmm. the sense strand comprises SEQ ID NO:130 and the antisense strand comprises SEQ ID NO:226;
nnnnnn. the sense strand comprises SEQ ID NO:130 and the antisense strand comprises SEQ ID NO:227;
oooooo. the sense strand comprises SEQ ID NO: 130 and the antisense strand comprises SEQ ID NO:228;
pppppp. the sense strand comprises SEQ ID NO: 131 and the antisense strand comprises SEQ ID NO:229;
qqqqqq. the sense strand comprises SEQ ID NO: 131 and the antisense strand comprises SEQ ID NO:230;
rrrrrr. the sense strand comprises SEQ ID NO: 131 and the antisense strand comprises SEQ ID NO:231;
ssssss. the sense strand comprises SEQ ID NO:232 and the antisense strand comprises SEQ ID NO: 184;
tttttt. the sense strand comprises SEQ ID NO:234 and the antisense strand comprises SEQ ID NO:215;
the sense strand comprises SEQ ID NO:236 and the antisense strand comprises SEQ ID NO:184;
vvvvvv. the sense strand comprises SEQ ID NO:237 and the antisense strand comprises SEQ ID NO:184;
wwwwww. the sense strand comprises SEQ ID NO:238 and the antisense strand comprises SEQ ID NO:213;
xxxxxx. the sense strand comprises SEQ ID NO:240 and the antisense strand comprises SEQ ID NO:213;
yyyyyy. the sense strand comprises SEQ ID NO:238 and the antisense strand comprises SEQ ID NO:215;
zzzzzz. the sense strand comprises SEQ ID NO:240 and the antisense strand comprises SEQ ID NO:215;
aaaaaaa. the sense strand comprises SEQ ID NO:241 and the antisense strand comprises SEQ ID NO:215;
bbbbbbb. the sense strand comprises SEQ ID NO:238 and the antisense strand comprises SEQ ID NO:212;
ccccccc. the sense strand comprises SEQ ID NO:232 and the antisense strand comprises SEQ ID NO:213;
ddddddd. the sense strand comprises SEQ ID NO:240 and the antisense strand comprises SEQ ID NO:214.

In some embodiments of the RNAi agents, the duplex comprises a sense strand and an antisense strand wherein:
a. the sense strand consists of SEQ ID NO:94 and the antisense strand consists of SEQ ID NO:132;
b. the sense strand consists of SEQ ID NO:95 and the antisense strand consists of SEQ ID NO:133;
c. the sense strand consists of SEQ ID NO:96 and the antisense strand consists of SEQ ID NO:134;
d. the sense strand consists of SEQ ID NO:97 and the antisense strand consists of SEQ ID NO:135;
e. the sense strand consists of SEQ ID NO:98 and the antisense strand consists of SEQ ID NO:136;
f. the sense strand consists of SEQ ID NO:99 and the antisense strand consists of SEQ ID NO: 137;
g. the sense strand consists of SEQ ID NO: 100 and the antisense strand consists of SEQ ID NO: 138;
h. the sense strand consists of SEQ ID NO: 101 the antisense strand consists of SEQ ID NO: 139;
i. the sense strand consists of SEQ ID NO: 102 and the antisense strand consists of SEQ ID NO:140;
j. the sense strand consists of SEQ ID NO: 103 and the antisense strand consists of SEQ ID NO:141;
k. the sense strand consists of SEQ ID NO: 104 and the antisense strand consists of SEQ ID NO: 142;
l. the sense strand consists of SEQ ID NO: 105 and the antisense strand consists of SEQ ID NO: 143;
m. the sense strand consists of SEQ ID NO: 106 and the antisense strand consists of SEQ ID NO:144;
n. the sense strand consists of SEQ ID NO: 107 and the antisense strand consists of SEQ ID NO: 145;
o. the sense strand consists of SEQ ID NO: 108 and the antisense strand consists of SEQ ID NO:146;
p. the sense strand consists of SEQ ID NO:109 and the antisense strand consists of SEQ ID NO:147;
q. the sense strand consists of SEQ ID NO:110 and the antisense strand consists of SEQ ID NO:148;
r. the sense strand consists of SEQ ID NO:111 and the antisense strand consists of SEQ ID NO:149;
s. the sense strand consists of SEQ ID NO:112 and the antisense strand consists of SEQ ID NO: 150;
t. the sense strand consists of SEQ ID NO: 113 and the antisense strand consists of SEQ ID NO:151;
u. the sense strand consists of SEQ ID NO:114 and the antisense strand consists of SEQ ID NO:152;
v. the sense strand consists of SEQ ID NO:115 and the antisense strand consists of SEQ ID NO:153;
w. the sense strand consists of SEQ ID NO: 116 and the antisense strand consists of SEQ ID NO:154;
x. the sense strand consists of SEQ ID NO:117 and the antisense strand consists of SEQ ID NO:155;
y. the sense strand consists of SEQ ID NO:118 and the antisense strand consists of SEQ ID NO:156;
z. the sense strand consists of SEQ ID NO:119 and the antisense strand consists of SEQ ID NO:157;
aa. the sense strand consists of SEQ ID NO: 120 and the antisense strand consists of SEQ ID NO:158;
bb. the sense strand consists of SEQ ID NO: 121 and the antisense strand consists of SEQ ID NO:159;
cc. the sense strand consists of SEQ ID NO: 122 and the antisense strand consists of SEQ ID NO:160;
dd. the sense strand consists of SEQ ID NO:123 and the antisense strand consists of SEQ ID NO:161;
ee. the sense strand consists of SEQ ID NO:96 and the antisense strand consists of SEQ ID NO:162;
ff. the sense strand consists of SEQ ID NO:96 and the antisense strand consists of SEQ ID NO:163;
gg. the sense strand consists of SEQ ID NO:96 and the antisense strand consists of SEQ ID NO:164;
hh. the sense strand consists of SEQ ID NO:96 and the antisense strand consists of SEQ ID NO:165;
ii. the sense strand consists of SEQ ID NO:96 and the antisense strand consists of SEQ ID NO:166;
jj. the sense strand consists of SEQ ID NO:124 and the antisense strand consists of SEQ ID NO: 162;
kk. the sense strand consists of SEQ ID NO:124 and the antisense strand consists of SEQ ID NO:163;
ll. the sense strand consists of SEQ ID NO:124 and the antisense strand consists of SEQ ID NO:164;
mm. the sense strand consists of SEQ ID NO:124 and the antisense strand consists of SEQ ID NO:165;
nn. the sense strand consists of SEQ ID NO: 124 and the antisense strand consists of SEQ ID NO:166;
oo. the sense strand consists of SEQ ID NO:124 and the antisense strand consists of SEQ ID NO:134;
pp. the sense strand consists of SEQ ID NO: 125 and the antisense strand consists of SEQ ID NO:162;
qq. the sense strand consists of SEQ ID NO:125 and the antisense strand consists of SEQ ID NO:163;
rr. the sense strand consists of SEQ ID NO: 125 and the antisense strand consists of SEQ ID NO:164;
ss. the sense strand consists of SEQ ID NO:125 and the antisense strand consists of SEQ ID NO:165;
tt. the sense strand consists of SEQ ID NO: 125 and the antisense strand consists of SEQ ID NO:166;
uu. the sense strand consists of SEQ ID NO: 125 and the antisense strand consists of SEQ ID NO:134;
vv. the sense strand consists of SEQ ID NO: 126 and the antisense strand consists of SEQ ID NO:162;
ww. the sense strand consists of SEQ ID NO:126 and the antisense strand consists of SEQ ID NO: 163;
xx. the sense strand consists of SEQ ID NO: 126 and the antisense strand consists of SEQ ID NO:164;
yy. the sense strand consists of SEQ ID NO: 126 and the antisense strand consists of SEQ ID NO:165;
zz. the sense strand consists of SEQ ID NO: 126 and the antisense strand consists of SEQ ID NO:166;
aaa. the sense strand consists of SEQ ID NO: 126 and the antisense strand consists of SEQ ID NO:134;
bbb. the sense strand consists of SEQ ID NO: 127 and the antisense strand consists of SEQ ID NO:167;
ccc. the sense strand consists of SEQ ID NO: 127 and the antisense strand consists of SEQ ID NO:168;
ddd. the sense strand consists of SEQ ID NO: 127 and the antisense strand consists of SEQ ID NO:169;
eee. the sense strand consists of SEQ ID NO:128 and the antisense strand consists of SEQ ID NO:170;
fff. the sense strand consists of SEQ ID NO: 128 and the antisense strand consists of SEQ ID NO:171;
ggg. the sense strand consists of SEQ ID NO: 128 and the antisense strand consists of SEQ ID NO:172;
hhh the sense strand consists of SEQ ID NO: 129 and the antisense strand consists of SEQ ID NO:173;
iii. the sense strand consists of SEQ ID NO:129 and the antisense strand consists of SEQ ID NO:174;
jjj. the sense strand consists of SEQ ID NO: 129 and the antisense strand consists of SEQ ID NO:175;
kkk. the sense strand consists of SEQ ID NO:130 and the antisense strand consists of SEQ ID NO:176;
lll. the sense strand consists of SEQ ID NO: 130 and the antisense strand consists of SEQ ID NO:177;
mmm. the sense strand consists of SEQ ID NO:130 and the antisense strand consists of SEQ ID NO:178;
nnn. the sense strand consists of SEQ ID NO: 131 and the antisense strand consists of SEQ ID NO:179;
ooo. the sense strand consists of SEQ ID NO:131 and the antisense strand consists of SEQ ID NO:180;
ppp. the sense strand consists of SEQ ID NO:131 and the antisense strand consists of SEQ ID NO:181;
qqq. the sense strand consists of SEQ ID NO:232 and the antisense strand consists of SEQ ID NO:233;
rrr. the sense strand consists of SEQ ID NO:234 and the antisense strand consists of SEQ ID NO:235;
sss. the sense strand consists of SEQ ID NO:236 and the antisense strand consists of SEQ ID NO:233;
ttt. the sense strand consists of SEQ ID NO:237 and the antisense strand consists of SEQ ID NO:233;
uuu. the sense strand consists of SEQ ID NO:238 and the antisense strand consists of SEQ ID NO:239;
vvv. the sense strand consists of SEQ ID NO:240 and the antisense strand consists of SEQ ID NO:239;
www. the sense strand consists of SEQ ID NO:238 and the antisense strand consists of SEQ ID NO:235;
xxx. the sense strand consists of SEQ ID NO:240 and the antisense strand consists of SEQ ID NO:235;
yyy. the sense strand consists of SEQ ID NO:241 and the antisense strand consists of SEQ ID NO:235;
zzz. the sense strand consists of SEQ ID NO:238 and the antisense strand consists of SEQ ID NO:242;
aaaa. the sense strand consists of SEQ ID NO:232 and the antisense strand consists of SEQ ID NO:239;
bbbb. the sense strand consists of SEQ ID NO:240 and the antisense strand consists of SEQ ID NO:242;
cccc. the sense strand consists of SEQ ID NO:94 and the antisense strand consists of SEQ ID NO: 182;
dddd. the sense strand consists of SEQ ID NO:95 and the antisense strand consists of SEQ ID NO: 183;
eeee. the sense strand consists of SEQ ID NO:96 and the antisense strand consists of SEQ ID NO: 184;
ffff. the sense strand consists of SEQ ID NO:97 and the antisense strand consists of SEQ ID NO: 185;
gggg. the sense strand consists of SEQ ID NO:98 and the antisense strand consists of SEQ ID NO: 186;
hhhh. the sense strand consists of SEQ ID NO:99 and the antisense strand consists of SEQ ID NO:187;
iiii. the sense strand consists of SEQ ID NO: 100 and the antisense strand consists of SEQ ID NO:188;
jjjj. the sense strand consists of SEQ ID NO: 101 and the antisense strand consists of SEQ ID NO:189;
kkkk. the sense strand consists of SEQ ID NO: 102 and the antisense strand consists of SEQ ID NO:190;
llll. the sense strand consists of SEQ ID NO: 103 and the antisense strand consists of SEQ ID NO:191;
mmmm. the sense strand consists of SEQ ID NO: 104 and the antisense strand consists of SEQ ID NO: 192;
nnnn. the sense strand consists of SEQ ID NO:105 and the antisense strand consists of SEQ ID NO:193;
oooo. the sense strand consists of SEQ ID NO:106 and the antisense strand consists of SEQ ID NO: 194;
pppp. the sense strand consists of SEQ ID NO:107 and the antisense strand consists of SEQ ID NO: 195;
qqqq. the sense strand consists of SEQ ID NO: 108 and the antisense strand consists of SEQ ID NO: 196;
rrrr. the sense strand consists of SEQ ID NO: 109 and the antisense strand consists of SEQ ID NO: 197;
ssss. the sense strand consists of SEQ ID NO:110 and the antisense strand consists of SEQ ID NO: 198;
tttt. the sense strand consists of SEQ ID NO:111 and the antisense strand consists of SEQ ID NO:199;
uuuu. the sense strand consists of SEQ ID NO:112 and the antisense strand consists of SEQ ID NO:200;
vvvv. the sense strand consists of o SEQ ID NO:113 and the antisense strand consists of SEQ ID NO:201;
wwww. the sense strand consists of SEQ ID NO:114 and the antisense strand consists of SEQ ID NO:202;
xxxx. the sense strand consists of SEQ ID NO:115 and the antisense strand consists of SEQ ID NO:203;
yyyy. the sense strand consists of SEQ ID NO:116 and the antisense strand consists of SEQ ID NO:204;
zzzz. the sense strand consists of SEQ ID NO:117 and the antisense strand consists of SEQ ID NO:205;
aaaaa. the sense strand consists of SEQ ID NO:118 and the antisense strand consists of SEQ ID NO:206;
bbbbb. the sense strand consists of SEQ ID NO:119 and the antisense strand consists of SEQ ID NO:207;
ccccc. the sense strand consists of SEQ ID NO: 120 and the antisense strand consists of SEQ ID NO:208;
ddddd. the sense strand consists of SEQ ID NO: 121 and the antisense strand consists of SEQ ID NO:209;
eeeee. the sense strand consists of SEQ ID NO: 122 and the antisense strand consists of SEQ ID NO:210;
fffff. the sense strand consists of SEQ ID NO:123 and the antisense strand consists of SEQ ID NO:211;
ggggg. the sense strand consists of SEQ ID NO:96 and the antisense strand consists of SEQ ID NO:212;
hhhhh. the sense strand consists of SEQ ID NO:96 and the antisense strand consists of SEQ ID NO:213;
iiiii. the sense strand consists of SEQ ID NO:96 and the antisense strand consists of SEQ ID NO:214;
the sense strand consists of SEQ ID NO:96 and the antisense strand consists of SEQ ID NO:215;
kkkkk. the sense strand consists of SEQ ID NO:96 and the antisense strand consists of SEQ ID NO:216;
lllll. the sense strand consists of SEQ ID NO:124 and the antisense strand consists of SEQ ID NO:212;
mmmmm. the sense strand consists of SEQ ID NO: 124 and the antisense strand consists of SEQ ID NO:213;
nnnnn. the sense strand consists of SEQ ID NO: 124 and the antisense strand consists of SEQ ID NO:214;
ooooo. the sense strand consists of SEQ ID NO: 124 and the antisense strand consists of SEQ ID NO:215;
ppppp. the sense strand consists of SEQ ID NO: 124 and the antisense strand consists of SEQ ID NO:216;
qqqqq. the sense strand consists of SEQ ID NO:124 and the antisense strand consists of SEQ ID NO: 184;
rrrrr. the sense strand consists of SEQ ID NO:125 and the antisense strand consists of SEQ ID NO:212;
sssss. the sense strand consists of SEQ ID NO:125 and the antisense strand consists of SEQ ID NO:213;
ttttt. the sense strand consists of SEQ ID NO:125 and the antisense strand consists of SEQ ID NO:214;
uuuuu. the sense strand comprises SEQ ID NO: 125 and the antisense strand comprises SEQ ID NO:215;
vvvvv. the sense strand consists of SEQ ID NO:125 and the antisense strand consists of SEQ ID NO:216;
wwwww. the sense strand consists of SEQ ID NO:125 and the antisense strand consists of SEQ ID NO: 184;
xxxxx. the sense strand consists of SEQ ID NO:126 and the antisense strand consists of SEQ ID NO:212;
yyyyy. the sense strand consists of SEQ ID NO:126 and the antisense strand consists of SEQ ID NO:213;
zzzzz. the sense strand consists of SEQ ID NO:126 and the antisense strand consists of SEQ ID NO:214;
aaaaaa. the sense strand consists of SEQ ID NO:126 and the antisense strand consists of SEQ ID NO:215;
bbbbbb. the sense strand consists of SEQ ID NO: 126 and the antisense strand consists of SEQ ID NO:216;
cccccc. the sense strand consists of SEQ ID NO:126 and the antisense strand consists of SEQ ID NO:184;
dddddd. the sense strand consists of SEQ ID NO:127 and the antisense strand consists of SEQ ID NO:217;
eeeeee. the sense strand consists of SEQ ID NO: 127 and the antisense strand consists of SEQ ID NO:218;
ffffff. the sense strand consists of SEQ ID NO:127 and the antisense strand consists of SEQ ID NO:219;
gggggg. the sense strand consists of SEQ ID NO:128 and the antisense strand consists of SEQ ID NO:220;
hhhhhh. the sense strand consists of SEQ ID NO: 128 and the antisense strand consists of SEQ ID NO:221;
iiiiii. the sense strand consists of SEQ ID NO:128 and the antisense strand consists of SEQ ID NO:222;
jjjjjj. the sense strand consists of SEQ ID NO:129 and the antisense strand consists of SEQ ID NO:223;
kkkkkk. the sense strand consists of SEQ ID NO: 129 and the antisense strand consists of SEQ ID NO:224;
llllll. the sense strand consists of SEQ ID NO:129 and the antisense strand consists of SEQ ID NO:225;
mmmmmm. the sense strand consists of SEQ ID NO:130 and the antisense strand consists of SEQ ID NO:226;
nnnnnn. the sense strand consists of SEQ ID NO:130 and the antisense strand consists of SEQ ID NO:227;
oooooo. the sense strand consists of SEQ ID NO:130 and the antisense strand consists of SEQ ID NO:228;
pppppp. the sense strand consists of SEQ ID NO:131 and the antisense strand consists of SEQ ID NO:229;
qqqqqq. the sense strand consists of SEQ ID NO: 131 and the antisense strand consists of SEQ ID NO:230;
rrrrrr. the sense strand consists of SEQ ID NO:131 and the antisense strand consists of SEQ ID NO:231;
ssssss. the sense strand consists of SEQ ID NO:232 and the antisense strand consists of SEQ ID NO: 184;
tttttt. the sense strand consists of SEQ ID NO:234 and the antisense strand consists of SEQ ID NO:215;
the sense strand consists of SEQ ID NO:236 and the antisense strand consists of SEQ ID NO:184;
vvvvvv. the sense strand consists of SEQ ID NO:237 and the antisense strand consists of SEQ ID NO: 184;
wwwwww. the sense strand consists of SEQ ID NO:238 and the antisense strand consists of SEQ ID NO:213;
xxxxxx. the sense strand consists of SEQ ID NO:240 and the antisense strand consists of SEQ ID NO:213;
yyyyyy. the sense strand consists of SEQ ID NO:238 and the antisense strand consists of SEQ ID NO:215;
zzzzzz. the sense strand consists of SEQ ID NO:240 and the antisense strand consists of SEQ ID NO:215;
aaaaaaa. the sense strand consists of SEQ ID NO:241 and the antisense strand consists of SEQ ID NO:215;
bbbbbbb. the sense strand consists of SEQ ID NO:238 and the antisense strand consists of SEQ ID NO:212;
ccccccc. the sense strand consists of SEQ ID NO:232 and the antisense strand consists of SEQ ID NO:213;
ddddddd. the sense strand consists of SEQ ID NO:240 and the antisense strand consists of SEQ ID NO:214.

In some embodiments of the RNAi agents the antisense strand consists essentially of SEQ ID NO: 233 and the sense strand consists essentially of SEQ ID NO: 232.

In some embodiments of the RNAi agents the antisense strand consists essentially of SEQ ID NO: 235 and the sense strand consists essentially of SEQ ID NO: 234.

In some embodiments of the RNAi agents the antisense strand consists essentially of SEQ ID NO: 233 and the sense strand consists essentially of or SEQ ID NO: 236.

In some embodiments, the RNAi agent comprises a sense strand that further comprises a delivery moiety. In some embodiments, the RNAi agent comprises an antisense strand that further comprises a delivery moiety.

In some embodiments of the RNAi agent, the delivery moiety is a lipophilic delivery moiety. In one embodiment, the deliver moiety is conjugated to a nucleobase in the sense strand as represented by Formula Ib. In one embodiment, the deliver moiety is conjugated to a nucleobase in the sense strand as represented by Formulae in Table 10. In some embodiments, the delivery moiety is conjugated to a nucleobase at position 12 or 13 from the 5′ strand of the sense strand.

In some embodiments, the RNAi agents comprise modifications of the nucleotides. Accordingly, in certain embodiments, the RNAi agents comprise modified nucleotides that are independently a 2′-fluoro modified nucleotide or a 2′-O-methyl modified nucleotide. In some embodiments, each nucleotide of the sense and each nucleotide of the antisense strand is modified.

In certain embodiments, the 2′ fluoro-modified nucleotides occur at particular positions, selected from the following:
a. Positions 2, 3, 7, 14, and 16 from the 5′ end of the antisense strand;
b. Positions 2, 5, 7, 14, and 16 from the 5′ end of the antisense strand;
c. Positions 2, 3, 8, 14, and 16 from the 5′ end of the antisense strand;
d. Positions 2, 5, 8, 14, and 16 from the 5′ end of the antisense strand;
e. Positions 2, 14 and 16 from the 5′ end of the antisense strand; or
f. Positions 2, 6, 14, and 16 from the 5′ end of the antisense strand.

In certain embodiments, the 2′ fluoro-modified nucleotides occur at particular positions, selected from the following:
a. positions 9, 10, and 11 from to the 5′-end of the sense strand;
b. positions 7, 9, and 11 from to the 5′-end of the sense strand;
c. positions 7, 9, and 10 from to the 5′-end of the sense strand;
d. positions 7, 10, and 11 from to the 5′-end of the sense strand; or
e. positions 7, 9, 10 and 11 from the 5′ end of the sense strand.

In further embodiments, the 2′ fluoro-modified nucleotides occur at particular positions of the antisense strand, selected from the following:
a. Positions 2, 3, 7, 14, and 16 from the 5′ end of the antisense strand;
b. Positions 2, 5, 7, 14, and 16 from the 5′ end of the antisense strand;
c. Positions 2, 3, 8, 14, and 16 from the 5′ end of the antisense strand;
d. Positions 2, 5, 8, 14, and 16 from the 5′ end of the antisense strand;
e. Positions 2, 14, and 16 from the 5′ end of the antisense strand; or
f. Positions 2, 6, 14, and 16 from the 5′ end of the antisense strand; and the 2′ fluoro-modified nucleotides occur at particular positions of the sense strand, selected from the following:
a. positions 9, 10, and 11 from to the 5′-end of the sense strand;
b. positions 7, 9, and 11 from to the 5′-end of the sense strand;
c. positions 7, 9, and 10 from to the 5′-end of the sense strand;
d. positions 7, 10, and 11 from to the 5′-end of the sense strand; or
e. positions 7, 9, 10 and 11 from the 5′ end of the sense strand; and
f. positions 7, 9, 10 and 11 from the 5′ end of the sense strand and positions 2, 6, 14, and 16 from the 5′ end of the antisense strand;
g. positions 9, 10, and 11 from to the 5′-end of the sense strand and positions 2, 5, 8, 14, and 16 from the 5′ end of the antisense strand.

In some embodiments of the RNAi agents, each nucleotide at positions 2, 6, 14, and 16 from the 5′ end of the antisense strand and positions 7, 9, 10, and 11 from the 5′ end of the sense strand comprises a 2′ fluoro modification.

In some embodiments of the RNAi agents, each nucleotide at positions 2, 5, 7, 14, and 16 from the 5′ end of the antisense strand and positions 9, 10, and 11 from the 5′ end of the sense strand comprises a 2′ fluoro modification;

In some embodiments of the RNAi agents, each nucleotide at positions 2, 3, 7, 14, and 16 from the 5′ end of the antisense strand and positions 9, 10, and 11 from the 5′ end of the sense strand comprises a 2′ fluoro modification.

In some embodiments of the RNAi agents, each nucleotide at positions 2, 5, 8, 14, and 16 from the 5′ end of the antisense strand and positions 9, 10, and 11 from the 5′ end of the sense strand comprises a 2′ fluoro modification.

In some embodiments of the RNAi agents, each nucleotide at positions 2, 14, and 16 from the 5′ end of the antisense strand and positions 9, 10, and 11 from the 5′ end of the sense strand comprises a 2′ fluoro modification.

In some embodiments of the RNAi agents, each nucleotide at positions 2, 5, 7, 14, and 16 from the 5′ end of the antisense strand and positions 7, 9, 10, and 11 from the 5′ end of the sense strand comprises a 2′ fluoro modification.

In some embodiments of the RNAi agents wherein nucleotides at specific positions are modified to comprise a 2′-fluoro modified nucleotide, the remaining nucleotides are also modified. In some embodiments of the RNAi agents wherein nucleotides at specific positions are modified to comprise a 2′-fluoro modified nucleotide, the remaining nucleotides are also modified, and optionally comprise a 2′-O-methyl modified nucleotide.

In some embodiments, the RNAi agents comprise one or more modified internucleoside linkages. In some embodiments, the internucleoside linkages are phosphorothioate linkages. In some embodiments, the antisense strand has four or five phosphorothioate linkages. In some embodiments, the sense strand has four or five phosphorothioate linkages. In some embodiments, the antisense strand comprises no more than four phosphorothioate internucleotide linkages. In some embodiments, positions 1 and 2 (e.g., relative to the 5′ end or 3′ end) of a sense strand are linked by modified internucleotide linkages. In some embodiments, positions 1 and 2 (e.g., relative to the 5′ end or 3′ end) of an antisense strand are linked by a phosphorothioate internucleotide linkage. In some embodiments, positions 2 and 3 (e.g., relative to the 5′ end or 3′ end) of a sense strand are linked by a modified internucleotide linkage. In some embodiments, positions 2 and 3 (e.g., relative to the 5′ end or 3′ end) of an antisense strand are linked by a phosphorothioate internucleotide linkage.

In certain embodiments, the 5′ end of the antisense strand is modified and comprises a phosphate group or a phosphate analog. In some embodiments, the phosphate analog is 5′-(E)-vinylphosphonate.

In some embodiments, the RNAi agent comprises a modified nucleotide that is an abasic moiety. In some embodiments, the sense strand has a modified nucleotide that is an abasic moiety. In some embodiments, the sense strand and the antisense strand of the RNAi agents herein form a region of complementarity of at least 15 nucleotides. In other embodiments, the sense strand and the antisense strand form a region of complementarity of at least 16, 17, or 18 nucleotides. In other embodiments, the sense strand and the antisense strand form a region of complementarity of 15, 16, 17, 18, 19, 20 or 21 nucleotides.

Another embodiment is an RNAi agent having a sense strand and an antisense strand, wherein the antisense strand is complementary to a portion of the mRNA of SEQ ID NO:1 (a transcript of the SCN10A gene), and wherein the sense strand and the antisense strand form a duplex, and wherein the sense strand and antisense strand are each independently 15 to 25 nucleotides in length, and optionally wherein the sense and antisense strand each independently comprise one or more modified nucleotides. In some embodiments, the sense strand and/or the antisense strand are selected from a Table disclosed herein. In some embodiments, the RNAi agent comprises an antisense strand and a sense strand forming a duplex selected from a Table disclosed herein.

Another embodiment disclosed herein is an RNAi agent having Formula I: R-L-D, wherein R is a double stranded oligonucleotide having a sense strand and an antisense strand, wherein the sense strand and the antisense strand form a region of complementarity; wherein D is a delivery moiety; and wherein L is either absent or a linker moiety; and wherein the antisense strand comprises at least 15 contiguous nucleotides complementary to the mRNA transcript of the SCN10A gene (e.g. SEQ ID NO: 1), and wherein the sense strand and the antisense strand are complementary across at least 15 nucleotides, and wherein in some embodiments, the sense strand and the antisense strand form a region of complementarity of 15, 16, 17, or 18 nucleotides, and wherein the sense strand and antisense strand are each independently 18 to 23 nucleotides in length, and optionally wherein the sense and antisense strand each independently comprise one or more modified nucleotides, and optionally D is.

In another embodiment disclosed herein is an RNAi agent having Formula I: R-L-D, wherein R is a double stranded oligonucleotide having a sense strand and an antisense strand, wherein the sense strand and the antisense strand form a duplex; wherein D is a delivery moiety; and wherein L is either absent or a linker moiety; and wherein the antisense strand comprises at least 15 contiguous nucleotides complementary to the mRNA transcript of the SCN10A gene, and wherein the sense strand and antisense strand are each independently 18 to 23 nucleotides in length, and optionally wherein the sense and antisense strand each independently comprise one or more modified nucleotides. In further embodiments, the antisense strand comprises at least 15, 16, 17, 18, 19, 20 or 21 contiguous nucleotides complementary to the SCN10A gene.

Also disclosed herein is an RNAi agent having Formula I: R-L-D, wherein R is a double stranded oligonucleotide having a sense strand and an antisense strand, wherein the sense strand and the antisense strand form a duplex; wherein D is a delivery moiety; and wherein L is either absent or a linker moiety; and wherein the antisense strand is complementary to at least 18 nucleotides of a sequence of Table 1, and wherein the sense strand and the antisense strand are complementary across at least 15, 16, 17, 18, 19, 20 or 21 nucleotides, and wherein the sense strand and antisense strand are each independently 18 to 23 nucleotides in length, and optionally wherein the sense and antisense strand each independently comprise one or more modified nucleotides.

In some embodiments, the delivery moiety is of Formula I:

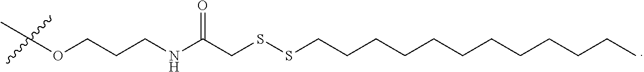 [SMILES: *OCCCNC(=O)CSSCCCCCCCCCCCC]

In some embodiments, the RNAi agents comprise a sense stand and an antisense strand, wherein the sense strand and the antisense strand form a duplex region, wherein the sense strand or the antisense strand comprises a modified nucleotide of the following Formula:

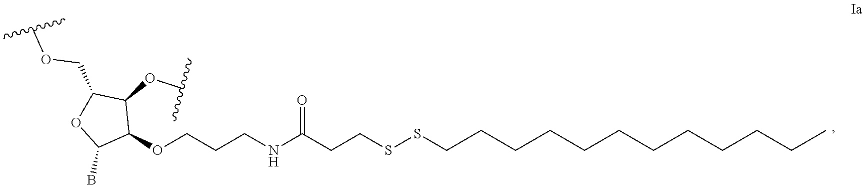

wherein in some embodiments B is a nucleobase selected from adenine (A), cytosine (C), guanine (G), thymine (T), uracil (U), or a derivative thereof. In some embodiments, B is a nucleobase selected from A, C, G, T, U. In some embodiments, B is a nucleobase derivative selected from 5-methyl cytosine, 2-thiouridine, 4-thiouridine, a C5-modified pyrimidine, C2-modified purine, N8-modified purine, a pseudouracil, isocytosine, isoguanine, 2,6-diamninopurine, a pseudocytosine, 2-aminopurine, xanthine, hypoxanthine, 7-methylguanine, 5-hydroxymethylcytosine, 5,6-dihydrouracil, 5-carboxy-cytidine, phenoxazine, N6-alkyl-A, or 06-alkyl-G.

In some embodiments, the sense strand comprises the modified nucleotide of Formula Ia. In some embodiments, the sense strand comprises the modified nucleotide of any one of Formula Ia, e.g., at any one of positions 1-6 or 12-21 from the 5′ end. In some embodiments, the sense strand comprises the modified nucleotide of Formula Ia, at position 13 from the 5′ end of the sense strand. In some embodiments, the sense strand comprises the modified nucleotide of Formula Ia, at position 12 from the 5′ end of the sense strand.

In some embodiments, the delivery moiety is conjugated to a nucleotide of the sense strand. In some embodiments, the delivery moiety is a modified nucleotide located in the sense strand. In some embodiments, the modified nucleotide is at position 12 or position 13 from the 5′ of the sense strand. In some embodiments, the modified nucleotide is 2′-O-hexadecyl uridine, 2′-O-hexadecyl cytidine, 2′-O-hexadecyl guanine, or 2′-O-hexadecyl adenosine depending on the sequence of the sense strand at position 12 or 13 (see Table 10 for structures).

For the RNAi agents described herein that decrease expression of the SCN10A gene, the antisense strand is complementary to a sequence of Table 1. In some embodiments, the antisense strand is complementary to at least 15 nucleotides of a sequence in Table 1. In some embodiments, the antisense strand is complementary to 18 contiguous nucleotides of a sequence of Table 1. In some embodiments, the RNAi agent comprises 0, 1, 2, or 3 mismatches between the sense strand and the antisense strand.

In some embodiments, the antisense strand or the sense strand of the RNAi agent has a sequence of at least 85%, 86%, 87%, 88%, 89%, 90%, 91%, 92%, 93%, 94%, 95%, 96%, 97%, 98%, or 99% sequence identity to an antisense strand or a sense strand in Table 2, Table 3A or 3B. In some embodiments, the antisense strand or the sense strand of the RNAi agent has 90%, 91%, 92%, 93%, 94%, 95%, 96%, 97%, 98%, 99% or 100% sequence identity to an antisense strand or a sense strand listed in Table 2, Table 3A or 3B.

In some embodiments, the sense and antisense strand are complementary across 15, 16, 17, 18, 19, or 20 nucleotides. In some embodiments, the sense and antisense strand are complementary across 15, 16, 17, 18, 19, or 20 contiguous nucleotides. In further embodiments, the RNAi agent comprises 0, 1, 2, or 3 mismatches between the sense strand and the antisense strand.

In some embodiments of the RNAi agents disclosed herein, the RNAi agent is capable of decreasing expression of the SCN10A gene in a cell. In some embodiments, the RNAi agents disclosed herein are for use in therapy. In one embodiment, the use is in the treatment of chronic pain. In some embodiments, the RNAi agents are for use in the manufacture of a medicament for the treatment of pain, including chronic pain.

In some embodiments, the RNAi agents are formulated in a pharmaceutical composition comprising the RNAi agent and one or more pharmaceutically acceptable excipients.

Another embodiment provided herein is a method of decreasing SCN10A protein and/or mRNA expression in a cell, comprising contacting the cell with an RNAi agent disclosed herein, and incubating the cell for a time sufficient for degrading SCN10A mRNA. Decreasing SCN10A protein and/or mRNA expression is measured by any suitable assay. In some embodiments, degrading SCN10A mRNA is determined by RT-PCR.

Some embodiments provided herein are methods of treating a patient that would benefit from decreasing expression of the mRNA transcript of the SCN10A gene, comprising administering an effective amount of the RNAi agent, or a pharmaceutical composition thereof, to a patient in need thereof. Some embodiments are methods of treating chronic pain in a patient in need thereof, comprising administering a therapeutic amount of the RNAi agent disclosed herein, or a pharmaceutical composition thereof.

The RNAi agents and compositions comprising RNAi agents are administered in any suitable way including without limitation: intrathecal delivery, e.g. intrathecal injection (lumbar puncture), epidural delivery, e.g. interlaminar (midline) epidural injection, transforaminal (peri-DRG) epidural injection, systemic delivery such as intravenous or subcutaneous delivery, e.g. intravenous or subcutaneous injection, or intra-ganglionic injection.

The dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In another aspect, provided herein are compounds, RNAi agents or pharmaceutical compositions for use in a therapy. Also provided herein are compounds, RNAi agents, or pharmaceutical compositions for use in the treatment of pain disorders.

| NCBI Reference Sequence: NM_006514.4 (SEQ ID NO: 1): | |
|---|---|
| SEQ ID NO: 1: | |
| 1 | ttcttgcttc acacactagc ctcttggcta gagaacagac atcagatgga gtttcttctg |
| 61 | gctatgcctg aatgttaagc tgaacgtatg ttccaggagc tcgtggtctc cagtagaggc |
| 121 | aatctgggat agaagagaag atatttctta cgtagaagac aagcaagatt gagcagcaaa |
| 181 | gagtataaat acttcctgaa gaagaatgag aagatggaat tccccattgg atccctcgaa |
| 241 | actaacaact tccgtcgctt tactccggag tcactggtgg agatagagaa gcaaattgct |
| 301 | gccaagcagg gaacaaagaa agccagagag aagcataggg agcagaagga ccaagaagag |
| 361 | aagcctcggc cccagctgga cttgaaagcc tgcaaccagc tgcccaagtt ctatggtgag |
| 421 | ctcccagcag aactgatcgg ggagcccctg gaggatctag atccgttcta cagcacacac |
| 481 | cggacattta tggtgctgaa caaagggagg accatttccc ggtttagtgc cactcgggcc |
| 541 | ctgtggctat tcagtccttt caacctgatc agaagaacgg ccatcaaagt gtctgtccac |
| 601 | tcgtggttca gtttatttat tacggtcact attttggtta attgtgtgtg catgacccga |
| 661 | actgaccttc cagagaaaat tgaatatgtc ttcactgtca tttacacctt tgaagccttg |
| 721 | ataaagatac tggcaagagg attttgtcta aatgagttca cgtacctgag agatccttgg |
| 781 | aactggctgg attttagcgt cattaccctg gcatatgttg gcacagcaat agatctccgt |
| 841 | gggatctcag gcctgcggac attcagagtt cttagagcat taaaaacagt ttctgtgatc |
| 901 | ccaggcctga aggtcattgt gggggccctg attcactcag tgaagaaact ggctgatgtg |
| 961 | accatcctca ccatcttctg cctaagtgtt tttgccttgg tggggctgca actcttcaag |
| 1021 | ggcaacctca aaaataaatg tgtcaagaat gacatggctg tcaatgagac aaccaactac |
| 1081 | tcatctcaca gaaaaccaga tatctacata aataagcgag gcacttctga ccccttactg |
| 1141 | tgtggcaatg gatctgactc aggccactgc cctgatggtt atatctgcct taaaacttct |
| 1201 | gacaacccgg attttaacta caccagcttt gattcctttg cttgggcttt cctctcactg |
| 1261 | ttccgcctca tgacacagga ttcctgggaa cgcctctacc agcagaccct gaggacttct |
| 1321 | gggaaaatct atatgatctt ttttgtgctc gtaatcttcc tgggatcttt ctacctggtc |
| 1381 | aacttgatct tggctgtagt caccatggcg tatgaggagc agaaccaggc aaccactgat |
| 1441 | gaaattgaag caaaggagaa gaagttccag gaggccctcg agatgctccg gaaggagcag |
| 1501 | gaggtgctag cagcactagg gattgacaca acctctctcc actcccacaa tggatcacct |
| 1561 | ttaacctcca aaaatgccag tgagagaagg catagaataa agccaagagt gtcagagggc |
| 1621 | tccacagaag acaacaaatc accccgctct gatccttaca accagcgcag gatgtctttt |
| 1681 | ctaggcctcg cctctggaaa acgccgggct agtcatggca gtgtgttcca tttccggtcc |
| 1741 | cctggccgag atatctcact ccctgaggga gtcacagatg atggagtctt tcctggagac |
| 1801 | cacgaaagcc atcggggctc tctgctgctg ggtgggggtg ctggccagca aggccccctc |
| 1861 | cctagaagcc ctcttcctca acccagcaac cctgactcca ggcatggaga agatgaacac |
| 1921 | caaccgccgc ccactagtga gcttgcccct ggagctgtcg atgtctcggc attcgatgca |
| 1981 | ggacaaaaga agactttctt gtcagcagaa tacttagatg aacctttccg ggcccaaagg |
| 2041 | gcaatgagtg ttgtcagtat cataacctcc gtccttgagg aactcgagga gtctgaacag |
| 2101 | aagtgcccac cctgcttgac cagcttgtct cagaagtatc tgatctggga ttgctgcccc |
| 2161 | atgtgggtga agctcaagac aattctcttt gggcttgtga cggatccctt tgcagagctc |
| 2221 | accatcacct tgtgcatcgt ggtgaacacc atcttcatgg ccatggagca ccatggcatg |
| 2281 | agccctacct tcgaagccat gctccagata ggcaacatcg tctttaccat attttttact |
| 2341 | gctgaaatgg tcttcaaaat cattgccttc gacccatact attatttcca gaagaagtgg |
| 2401 | aatatctttg actgcatcat cgtcactgtg agtctgctag agctgggcgt ggccaagaag |
| 2461 | ggaagcctgt ctgtgctgcg gagcttccgc ttgctgcgcg tattcaagct ggccaaatcc |
| 2521 | tggcccacct taaacacact catcaagatc atcggaaact cagtgggggc actggggaac |
| 2581 | ctcaccatca tcctggccat cattgtcttt gtctttgctc tggttggcaa gcagctccta |
| 2641 | ggggaaaact accgtaacaa ccgaaaaaat atctccgcgc cccatgaaga ctggccccgc |
| 2701 | tggcacatgc acgacttctt ccactctttc ctcattgtct tccgtatcct ctgtggagag |
| 2761 | tggattgaga acatgtgggc ctgcatggaa gttggccaaa aatccatatg cctcatcctt |
| 2821 | ttcttgacgg tgatggtgct agggaacctg gtggtgctta acctgttcat cgccctgcta |
| 2881 | ttgaactctt tcagtgctga caacctcaca gccccggagg acgatgggga ggtgaacaac |
| 2941 | ctgcaggtgg ccctggcacg gatccaggtc tttggccatc gtaccaaaca ggctctttgc |
| 3001 | agcttcttca gcaggtcctg cccattcccc cagcccaagg cagagcctga gctggtggtg |
| 3061 | aaactcccac tctccagctc caaggctgag aaccacattg ctgccaacac tgccaggggg |
| 3121 | agctctggag ggctccaagc tcccagaggc cccagggatg agcacagtga cttcatcgct |
| 3181 | aatccgactg tgtgggtctc tgtgcccatt gctgagggtg aatctgatct tgatgacttg |
| 3241 | gaggatgatg gtggggaaga tgctcagagc ttccagcagg aagtgatccc caaaggacag |
| 3301 | caggagcagc tgcagcaagt cgagaggtgt ggggaccacc tgacacccag gagcccaggc |
| 3361 | actggaacat cttctgagga cctggctcca tccctgggtg agacgtggaa agatgagtct |
| 3421 | gttcctcagg tccctgctga gggagtggac gacacaagct cctctgaggg cagcacggtg |
| 3481 | gactgcctag atcctgagga aatcctgagg aagatccctg agctggcaga tgacctggaa |
| 3541 | gaaccagatg actgcttcac agaaggatgc attcgccact gtccctgctg caaactggat |
| 3601 | accaccaaga gtccatggga tgtgggctgg caggtgcgca agacttgcta ccgtatcgtg |
| 3661 | gagcacagct ggtttgagag cttcatcatc ttcatgatcc tgctcagcag tggatctctg |
| 3721 | gcctttgaag actattacct ggaccagaag cccacggtga aagctttgct ggagtacact |
| 3781 | gacagggtct tcacctttat ctttgtgttc gagatgctgc ttaagtgggt ggcctatggc |
| 3841 | ttcaaaaagt acttcaccaa tgcctggtgc tggctggact tcctcattgt gaatatctca |
| 3901 | ctgataagtc tcacagcgaa gattctggaa tattctgaag tggctcccat caaagccctt |
| 3961 | cgaacccttc gcgctctgcg gccactgcgg gctctttctc gatttgaagg catgcgggtg |
| 4021 | gtggtggatg ccctggtggg cgccatccca tccatcatga atgtcctcct cgtctgcctc |
| 4081 | atcttctggc tcatcttcag catcatgggt gtgaacctct tcgcagggaa gttttggagg |
| 4141 | tgcatcaact ataccgatgg agagttttcc cttgtacctt tgtcgattgt gaataacaag |
| 4201 | tctgactgca agattcaaaa ctccactggc agcttcttct gggtcaatgt gaaagtcaac |
| 4261 | tttgataatg ttgcaatggg ttaccttgca cttctgcagg tggcaacctt taaaggctgg |
| 4321 | atggacatta tgtatgcagc tgttgattcc cgggaggtca acatgcaacc caagtgggag |
| 4381 | gacaacgtgt acatgtattt gtactttgtc atcttcatca tttttggagg cttcttcaca |
| 4441 | ctgaatctct ttgttggggt cataattgac aacttcaatc aacagaaaaa aaagttaggg |
| 4501 | ggccaggaca tcttcatgac agaggagcag aagaaatact acaatgccat gaagaagttg |
| 4561 | ggctccaaga agccccagaa gcccatccca cggcccctga acaagttcca gggttttgtc |
| 4621 | tttgacatcg tgaccagaca agcttttgac atcaccatca tggtcctcat ctgcctcaac |
| 4681 | atgatcacca tgatggtgga gactgatgac caaagtgaag aaaagacgaa aattctgggc |
| 4741 | aaaatcaacc agttctttgt ggccgtcttc acaggcgaat gtgtcatgaa gatgttcgct |
| 4801 | ttgaggcagt actacttcac aaatggctgg aatgtgtttg acttcattgt ggtggttctc |
| 4861 | tccattgcga gcctgatttt ttctgcaatt cttaagtcac ttcaaagtta cttctcccca |
| 4921 | acgctcttca gagtcatccg cctggcccga attggccgca tcctcagact gatccgagcg |
| 4981 | gccaagggga tccgcacact gctctttgcc ctcatgatgt ccctgcctgc cctcttcaac |
| 5041 | atcgggctgt tgctattcct tgtcatgttc atctactcta tcttcggtat gtccagcttt |
| 5101 | ccccatgtga ggtgggaggc tggcatcgac gacatgttca acttccagac cttcgccaac |
| 5161 | agcatgctgt gcctcttcca gattaccacg tcggccggct gggatggcct cctcagcccc |
| 5221 | atcctcaaca cagggccccc ctactgtgac cccaatctgc ccaacagcaa tggcaccaga |
| 5281 | ggggactgtg ggagcccagc cgtaggcatc atcttcttca ccacctacat catcatctcc |
| 5341 | ttcctcatca tggtcaacat gtacattgca gtgattctgg agaacttcaa tgtggccacg |
| 5401 | gaggagagca ctgagcccct gagtgaggac gactttgaca tgttctatga gacctgggag |
| 5461 | aagtttgacc cagaggccac tcagtttatt accttttctg ctctctcgga ctttgcagac |
| 5521 | actctctctg gtcccctgag aatcccaaaa cccaatcgaa atatactgat ccagatggac |
| 5581 | ctgcctttgg tccctggaga taagatccac tgcttggaca tcctttttgc tttcaccaag |
| 5641 | aatgtcctag gagaatccgg ggagttggat tctctgaagg caaatatgga ggagaagttt |
| 5701 | atggcaacta atctttcaaa atcatcctat gaaccaatag caaccactct ccgatggaag |
| 5761 | caagaagaca tttcagccac tgtcattcaa aaggcctatc ggagctatgt gctgcaccgc |
| 5821 | tccatggcac tctctaacac cccatgtgtg cccagagctg aggaggaggc tgcatcactc |
| 5881 | ccagatgaag gttttgttgc attcacagca aatgaaaatt gtgtactccc agacaaatct |
| 5941 | gaaactgctt ctgccacatc attcccaccg tcctatgaga gtgtcactag aggccttagt |
| 6001 | gatagagtca acatgaggac atctagctca atacaaaatg aagatgaagc caccagtatg |
| 6061 | gagctgattg cccctgggcc ctagtgagaa cactccagcc tggatatgtt cagttatgat |
| 6121 | gcgtccttct gctctgtgtt aactctttcc cattgcagat cacacccagc tacagcctca |
| 6181 | ccaatgcatg ccactggtca caatgtcaga actgggcagg gaatgcaact ggtaaccacc |
| 6241 | tttgaaaaaa ccatcataca caaataggag tcagaagcta agagcacttc cactgtgatt |
| 6301 | tcccttctga atttcaatat cagatcatgg gttctcttac tctccagaaa atatcccttg |
| 6361 | tccttttatt tttgttgtaa tcagattata tttactgaga caaacttctc aggaccagaa |
| 6421 | cttccaaaga tatagaacaa gaacattact gaaggtccaa ggtagtcctc tgtggagcac |
| 6481 | catacagaga ttggcaatat tatttaggat tttgcatgac tgcatgtgag agctgtcgga |
| 6541 | caactgctct gacccagggt aacacctgtg gcctatgtca tgaaaagcct ttgagatatc |
| 6601 | cattaaaatt aatattttta aatgta |

TABLE 1
Target regions of SCN10A gene

| SEQ ID NO: | 18 mer Target Regions |
|---|---|
| SEQ ID NO: 2 | UAACUACACCAGCUUUGA |
| SEQ ID NO: 3 | UUGUCAGCAGAAUACUUA |
| SEQ ID NO: 4 | UCAGCAGAAUACUUAGAU |
| SEQ ID NO: 5 | GUGACUUCAUCGCUAAUC |
| SEQ ID NO: 6 | GCACAGUGACUUCAUCGC |
| SEQ ID NO: 7 | UGUCUAAAUGAGUUCACG |
| SEQ ID NO: 8 | UUUGAAGCCUUGAUAAAG |
| SEQ ID NO: 9 | AUCCUAUGAACCAAUAGC |
| SEQ ID NO: 10 | ACUCUUUCAGUGCUGACA |
| SEQ ID NO: 11 | GAAUCUCUUUGUUGGGGU |
| SEQ ID NO: 12 | AUGAGGACAUCUAGCUCA |
| SEQ ID NO: 13 | UUACGGUCACUAUUUUGG |
| SEQ ID NO: 14 | CAAUUCUCUUUGGGCUUG |
| SEQ ID NO: 15 | CAUGAGCCCUACCUUCGA |
| SEQ ID NO: 16 | CAAUGAGUGUUGUCAGUA |
| SEQ ID NO: 17 | UUGGCAAGCAGCUCCUAG |
| SEQ ID NO: 18 | UCACCAUCUUCUGCCUAA |
| SEQ ID NO: 19 | CAGAUGAUGGAGUCUUUC |
| SEQ ID NO: 20 | GGCAUAUGUUGGCACAGC |
| SEQ ID NO: 21 | AGUGUUGUCAGUAUCAUA |
| SEQ ID NO: 22 | CUAUGAACCAAUAGCAAC |
| SEQ ID NO: 23 | UGUUGUCAGUAUCAUAAC |
| SEQ ID NO: 24 | AUAACAAGUCUGACUGCA |
| SEQ ID NO: 25 | UCAUUACCCUGGCAUAUG |
| SEQ ID NO: 26 | ACUGACAGGGUCUUCACC |
| SEQ ID NO: 27 | AAGCCUUGAUAAAGAUAC |
| SEQ ID NO: 28 | UGAGAGUGUCACUAGAGG |
| SEQ ID NO: 29 | AUUUCAGCCACUGUCAUU |
| SEQ ID NO: 30 | GAUUGUGAAUAACAAGUC |
| SEQ ID NO: 31 | UACGGUCACUAUUUUGGU |
| SEQ ID NO: 32 | UCAGCAG-Ab-AUACUUAGAU |

TABLE 2
Exemplary nucleotide sequences of the sense and antisense strands of
RNAi agents that decrease expression of the mRNA transcript of the SCN10A gene:

| Duplex | SEQ ID | | SEQ ID | |
|---|---|---|---|---|
| ID NO: | NO: | Sense (5′ to 3′) | NO: | Antisense (5′-3′) |
| Duplex | SEQ ID | UUUAACUACACCAGCUUUGAA | SEQ ID | UUCAAAGCUGGUGUAGUUAAAAU |
| ID NO: 1 | NO: 33 | | NO: 64 | |
| Duplex | SEQ ID | UCUUGUCAGCAGAAUACUUAA | SEQ ID | UUAAGUAUUCUGCUGACAAGAAA |
| ID NO: 2 | NO: 34 | | NO: 65 | |
| Duplex | SEQ ID | UGUCAGCAGAAUACUUAGAUA | SEQ ID | UAUCUAAGUAUUCUGCUGACAAG |
| ID NO: 3 | NO: 35 | | NO: 66 | |
| Duplex | SEQ ID | CAGUGACUUCAUCGCUAAUCA | SEQ ID | UGAUUAGCGAUGAAGUCACUGUG |
| ID NO: 4 | NO: 36 | | NO: 67 | |
| Duplex | SEQ ID | GAGCACAGUGACUUCAUCGCA | SEQ ID | UGCGAUGAAGUCACUGUGCUCAU |
| ID NO: 5 | NO: 37 | | NO: 68 | |
| Duplex | SEQ ID | UUUGUCUAAAUGAGUUCACGA | SEQ ID | UCGUGAACUCAUUUAGACAAAAU |
| ID NO: 6 | NO: 38 | | NO: 69 | |
| Duplex | SEQ ID | CCUUUGAAGCCUUGAUAAAGA | SEQ ID | UCUUUAUCAAGGCUUCAAAGGUG |
| ID NO: 7 | NO: 39 | | NO: 70 | |
| Duplex | SEQ ID | UCAUCCUAUGAACCAAUAGCA | SEQ ID | UGCUAUUGGUUCAUAGGAUGAUU |
| ID NO: 8 | NO: 40 | | NO: 71 | |
| Duplex | SEQ ID | GAACUCUUUCAGUGCUGACAA | SEQ ID | UUGUCAGCACUGAAAGAGUUCAA |
| ID NO: 9 | NO: 41 | | NO: 72 | |
| Duplex | SEQ ID | CUGAAUCUCUUUGUUGGGGUA | SEQ ID | UACCCCAACAAAGAGAUUCAGUG |
| ID NO: | NO: 42 | | NO: 73 | |
| 10 | | | | |
| Duplex | SEQ ID | ACAUGAGGACAUCUAGCUCAA | SEQ ID | UUGAGCUAGAUGUCCUCAUGUUG |
| ID NO: | NO: 43 | | NO: 74 | |
| 11 | | | | |
| Duplex | SEQ ID | UAUUACGGUCACUAUUUUGGA | SEQ ID | UCCAAAAUAGUGACCGUAAUAAA |
| ID NO: | NO: 44 | | NO: 75 | |
| 12 | | | | |
| Duplex | SEQ ID | GACAAUUCUCUUUGGGCUUGA | SEQ ID | UCAAGCCCAAAGAGAAUUGUCUU |
| ID NO: | NO: 45 | | NO: 76 | |
| 13 | | | | |
| Duplex | SEQ ID | GGCAUGAGCCCUACCUUCGAA | SEQ ID | UUCGAAGGUAGGGCUCAUGCCAU |
| ID NO: | NO: 46 | | NO: 77 | |
| 14 | | | | |
| Duplex | SEQ ID | GGCAAUGAGUGUUGUCAGUAA | SEQ ID | UUACUGACAACACUCAUUGCCCU |
| ID NO: | NO: 47 | | NO: 78 | |
| 15 | | | | |
| Duplex | SEQ ID | GGUUGGCAAGCAGCUCCUAGA | SEQ ID | UCUAGGAGCUGCUUGCCAACCAG |
| ID NO: | NO: 48 | | NO: 79 | |
| 16 | | | | |
| Duplex | SEQ ID | CCUCACCAUCUUCUGCCUAAA | SEQ ID | UUUAGGCAGAAGAUGGUGAGGAU |
| ID NO: | NO: 49 | | NO: 80 | |
| 17 | | | | |
| Duplex | SEQ ID | CACAGAUGAUGGAGUCUUUCA | SEQ ID | UGAAAGACUCCAUCAUCUGUGAC |
| ID NO: | NO: 50 | | NO: 81 | |
| 18 | | | | |
| Duplex | SEQ ID | CUGGCAUAUGUUGGCACAGCA | SEQ ID | UGCUGUGCCAACAUAUGCCAGGG |
| ID NO: | NO: 51 | | NO: 82 | |
| 19 | | | | |
| Duplex | SEQ ID | UGAGUGUUGUCAGUAUCAUAA | SEQ ID | UUAUGAUACUGACAACACUCAUU |
| ID NO: | NO: 52 | | NO: 83 | |
| 20 | | | | |
| Duplex | SEQ ID | UCCUAUGAACCAAUAGCAACA | SEQ ID | UGUUGCUAUUGGUUCAUAGGAUG |
| ID NO: | NO: 53 | | NO: 84 | |
| 21 | | | | |
| Duplex | SEQ ID | AGUGUUGUCAGUAUCAUAACA | SEQ ID | UGUUAUGAUACUGACAACACUCA |
| ID NO: | NO: 54 | | NO: 85 | |
| 22 | | | | |
| Duplex | SEQ ID | GAAUAACAAGUCUGACUGCAA | SEQ ID | UUGCAGUCAGACUUGUUAUUCAC |
| ID NO: | NO: 55 | | NO: 86 | |
| 23 | | | | |
| Duplex | SEQ ID | CGUCAUUACCCUGGCAUAUGA | SEQ ID | UCAUAUGCCAGGGUAAUGACGCU |
| ID NO: | NO: 56 | | NO: 87 | |
| 24 | | | | |
| Duplex | SEQ ID | ACACUGACAGGGUCUUCACCA | SEQ ID | UGGUGAAGACCCUGUCAGUGUAC |
| ID NO: | NO: 57 | | NO: 88 | |
| 25 | | | | |
| Duplex | SEQ ID | UGAAGCCUUGAUAAAGAUACA | SEQ ID | UGUAUCUUUAUCAAGGCUUCAAA |
| ID NO: | NO: 58 | | NO: 89 | |
| 26 | | | | |
| Duplex | SEQ ID | UAUGAGAGUGUCACUAGAGGA | SEQ ID | UCCUCUAGUGACACUCUCAUAGG |
| ID NO: | NO: 59 | | NO: 90 | |
| 27 | | | | |
| Duplex | SEQ ID | ACAUUUCAGCCACUGUCAUUA | SEQ ID | UAAUGACAGUGGCUGAAAUGUCU |
| ID NO: | NO: 60 | | NO: 91 | |
| 28 | | | | |
| Duplex | SEQ ID | UCGAUUGUGAAUAACAAGUCA | SEQ ID | UGACUUGUUAUUCACAAUCGACA |
| ID NO: | NO: 61 | | NO: 92 | |
| 29 | | | | |
| Duplex | SEQ ID | AUUACGGUCACUAUUUUGGUA | SEQ ID | UACCAAAAUAGUGACCGUAAUAA |
| ID NO: | NO: 62 | | NO: 93 | |
| 30 | | | | |
| Duplex | SEQ ID | UGUCAGCAG-Ab- | SEQ ID | UAUCUAAGUAUUCUGCUGACAAG |
| ID NO: | NO: 63 | AUACUUAGAUA | NO: 66 | |
| 31 | | | | |

TABLE 3A
Listing of one embodiment of SCN10A RNAi agents. In some
embodiments, the RNAi agents are conjugated to a delivery moiety. In one
embodiment the delivery moiety is teg-cholesterol.

| Duplex ID | | |
|---|---|---|
| NO: | SEQ ID NO: | Modified (5′ to 3′) |
| Duplex ID | SEQ ID NO: 94 | mU*mU*mUmAmAmCfUmAfCfAfCmCmAmGmCmUm |
| NO: 32 | | UmUmG*mA*mA |
| | SEQ ID NO: | PmU*fU*mCmAmAfAmGmCmUmGmGmUmGfUmAfG |
| | 132 | mUmUmAmAmA*mA*mU |
| Duplex ID | SEQ ID NO: 95 | mU*mC*mUmUmGmUfCmAfGfCfAmGmAmAmUmAm |
| NO: 33 | | CmUmU*mA*mA |
| | SEQ ID NO: | PmU*fU*mAmAmGfUmAmUmUmCmUmGmCfUmGfA |
| | 133 | mCmAmAmGmA*mA*mA |
| Duplex ID | SEQ ID NO: 96 | mU*mG*mUmCmAmGfCmAfGfAfAmUmAmCmUmUm |
| NO: 34 | | AmGmA*mU*mA |
| | SEQ ID NO: | PmU*fA*mUmCmUfAmAmGmUmAmUmUmCfUmGfC |
| | 134 | mUmGmAmCmA*mA*mG |
| Duplex ID | SEQ ID NO: 97 | mC*mA*mGmUmGmAfCmUfUfCfAmUmCmGmCmUm |
| NO: 35 | | AmAmU*mC*mA |
| | SEQ ID NO: | PmU*fG*mAmUmUfAmGmCmGmAmUmGmAfAmGfU |
| | 135 | mCmAmCmUmG*mU*mG |
| Duplex ID | SEQ ID NO: 98 | mG*mA*mGmCmAmCfAmGfUfGfAmCmUmUmCmAm |
| NO: 36 | | UmCmG*mC*mA |
| | SEQ ID NO: | PmU*fG*mCmGmAfUmGmAmAmGmUmCmAfCmUfG |
| | 136 | mUmGmCmUmC*mA*mU |
| Duplex ID | SEQ ID NO: 99 | mU*mU*mUmGmUmCfUmAfAfAfUmGmAmGmUmUm |
| NO: 37 | | CmAmC*mG*mA |
| | SEQ ID NO: | PmU*fC*mGmUmGfAmAmCmUmCmAmUmUfUmAfG |
| | 137 | mAmCmAmAmA*mA*mU |
| Duplex ID | SEQ ID NO: | mC*mC*mUmUmUmGfAmAfGfCfCmUmUmGmAmUm |
| NO: 38 | 100 | AmAmA*mG*mA |
| | SEQ ID NO: | PmU*fC*mUmUmUfAmUmCmAmAmGmGmCfUmUfC |
| | 138 | mAmAmAmGmG*mU*mG |
| Duplex ID | SEQ ID NO: | mU*mC*mAmUmCmCfUmAfUfGfAmAmCmCmAmAm |
| NO: 39 | 101 | UmAmG*mC*mA |
| | SEQ ID NO: | PmU*fG*mCmUmAfUmUmGmGmUmUmCmAfUmAfG |
| | 139 | mGmAmUmGmA*mU*mU |
| Duplex ID | SEQ ID NO: | mG*mA*mAmCmUmCfUmUfUfCfAmGmUmGmCmUm |
| NO: 40 | 102 | GmAmC*mA*mA |
| | SEQ ID NO: | PmU*fU*mGmUmCfAmGmCmAmCmUmGmAfAmAfG |
| | 140 | mAmGmUmUmC*mA*mA |
| Duplex ID | SEQ ID NO: | mC*mU*mGmAmAmUfCmUfCfUfUmUmGmUmUmGm |
| NO: 41 | 103 | GmGmG*mU*mA |
| | SEQ ID NO: | PmU*fA*mCmCmCfCmAmAmCmAmAmAmGfAmGfA |
| | 141 | mUmUmCmAmG*mU*mG |
| Duplex ID | SEQ ID NO: | mA*mC*mAmUmGmAfGmGfAfCfAmUmCmUmAmGm |
| NO: 42 | 104 | CmUmC*mA*mA |
| | SEQ ID NO: | PmU*fU*mGmAmGfCmUmAmGmAmUmGmUfCmCfU |
| | 142 | mCmAmUmGmU*mU*mG |
| Duplex ID | SEQ ID NO: | mU*mA*mUmUmAmCfGmGfUfCfAmCmUmAmUmUm |
| NO: 43 | 105 | UmUmG*mG*mA |
| | SEQ ID NO: | PmU*fC*mCmAmAfAmAmUmAmGmUmGmAfCmCfG |
| | 143 | mUmAmAmUmA*mA*mA |
| Duplex ID | SEQ ID NO: | mG*mA*mCmAmAmUfUmCfUfCfUmUmUmGmGmGm |
| NO: 44 | 106 | CmUmU*mG*mA |
| | SEQ ID NO: | PmU*fC*mAmAmGfCmCmCmAmAmAmGmAfGmAfA |
| | 144 | mUmUmGmUmC*mU*mU |
| Duplex ID | SEQ ID NO: | mG*mG*mCmAmUmGfAmGfCfCfCmUmAmCmCmUm |
| NO: 45 | 107 | UmCmG*mA*mA |
| | SEQ ID NO: | PmU*fU*mCmGmAfAmGmGmUmAmGmGmGfCmUfC |
| | 145 | mAmUmGmCmC*mA*mU |
| Duplex ID | SEQ ID NO: | mG*mG*mCmAmAmUfGmAfGfUfGmUmUmGmUmCm |
| NO: 46 | 108 | AmGmU*mA*mA |
| | SEQ ID NO: | PmU*fU*mAmCmUfGmAmCmAmAmCmAmCfUmCfA |
| | 146 | mUmUmGmCmC*mC*mU |
| Duplex ID | SEQ ID NO: | mG*mG*mUmUmGmGfCmAfAfGfCmAmGmCmUmCm |
| NO: 47 | 109 | CmUmA*mG*mA |
| | SEQ ID NO: | PmU*fC*mUmAmGfGmAmGmCmUmGmCmUfUmGfC |
| | 147 | mnCmAmAmCmC*mA*mG |
| Duplex ID | SEQ ID NO: | mC*mC*mUmCmAmCfCmAfUfCfUmUmCmUmGmCm |
| NO: 48 | 110 | CmUmA*mA*mA |
| | SEQ ID NO: | PmU*fU*mUmAmGfGmCmAmGmAmAmGmAfUmGfG |
| | 148 | mUmGmAmGmG*mA*mU |
| Duplex ID | SEQ ID NO: | mC*mA*mCmAmGmAfUmGfAfUfGmGmAmGmUmCm |
| NO: 49 | 111 | UmUmU*mC*mA |
| | SEQ ID NO: | PmU*fG*mAmAmAfGmAmCmUmCmCmAmUfCmAfU |
| | 149 | mCmUmGmUmG*mA*mC |
| Duplex ID | SEQ ID NO: | mC*mU*mGmGmCmAfUmAfUfGfUmUmGmGmCmAm |
| NO: 50 | 112 | CmAmG*mC*mA |
| | SEQ ID NO: | PmU*fG*mCmUmGfUmGmCmCmAmAmCmAfUmAfU |
| | 150 | mGmCmCmAmG*mG*mG |
| Duplex ID | SEQ ID NO: | mU*mG*mAmGmUmGfUmUfGfUfCmAmGmUmAmUm |
| NO: 51 | 113 | CmAmU*mA*mA |
| | SEQ ID NO: | PmU*fU*mAmUmGfAmUmAmCmUmGmAmCfAmAfC |
| | 151 | mAmCmUmCmA*mU*mU |
| Duplex ID | SEQ ID NO: | mU*mC*mCmUmAmUfGmAfAfCfCmAmAmUmAmGm |
| NO: 52 | 114 | CmAmA*mC*mA |
| | SEQ ID NO: | PmU*fG*mUmUmGfCmUmAmUmUmGmGmUfUmCfA |
| | 152 | mUmAmGmGmA*mU*mG |
| Duplex ID | SEQ ID NO: | mA*mG*mUmGmUmUfGinUfCfAfGmUmAmUmCmAm |
| NO: 53 | 115 | UmAmA*mC*mA |
| | SEQ ID NO: | PmU*fG*mUmUmAfUmGmAmUmAmCmUmGfAmCfA |
| | 153 | mAmCmAmCmU*mC*mA |
| Duplex ID | SEQ ID NO: | mG*mA*mAmUmAmAfCmAfAfGfUmCmUmGmAmCm |
| NO: 54 | 116 | UmGmC*mA*mA |
| | SEQ ID NO: | PmU*fU*mGmCmAfGmUmCmAmGmAmCmUfUmGfU |
| | 154 | mUmAmUmUmC*mA*mC |
| Duplex ID | SEQ ID NO: | mC*mG*mUmCmAmUfUmAfCfCfCmUmGmGmCmAm |
| NO: 55 | 117 | UmAmU*mG*mA |
| | SEQ ID NO: | PmU*fC*mAmUmAfUmGmCmCmAmGmGmGfUmAfA |
| | 155 | mUmGmAmCmG*mC*mU |
| Duplex ID | SEQ ID NO: | mA*mC*mAmCmUmGfAmCfAfGfGmGmUmCmUmUm |
| NO: 56 | 118 | CmAmC*mC*mA |
| | SEQ ID NO: | PmU*fG*mGmUmGfAmAmGmAmCmCmCmUfGmUfC |
| | 156 | mAmGmUmGmU*mA*mC |
| Duplex ID | SEQ ID NO: | mU*mG*mAmAmGmCfCmUfUfGfAmUmAmAmAmGm |
| NO: 57 | 119 | AmUmA*mC*mA |
| | SEQ ID NO: | PmU*fG*mUmAmUfCmUmUmUmAmUmCmAfAmGfG |
| | 157 | mCmUmUmCmA*mA*mA |
| Duplex ID | SEQ ID NO: | mU*mA*mUmGmAmGfAmGfUfGfUmCmAmCmUmAm |
| NO: 58 | 120 | GmAmG*mG*mA |
| | SEQ ID NO: | PmU*fC*mCmUmCfUmAmGmUmGmAmCmAfCmUfC |
| | 158 | mUmCmAmUmA*mG*mG |
| Duplex ID | SEQ ID NO: | mA*mC*mAmUmUmUfCmAfGfCfCmAmCmUmGmUm |
| NO: 59 | 121 | CmAmU*mU*mA |
| | SEQ ID NO: | PmU*fAmAmUmGfAmCmAmGmUmGmGmCfUmGfA |
| | 159 | mAmAmUmGmU*mC*mU |
| Duplex ID | SEQ ID NO: | mU*mC*mGmAmUmUfGmUfGfAfAmUmAmAmCmAm |
| NO: 60 | 122 | AmGmU*mC*mA |
| | SEQ ID NO: | PmU*fG*mAmCmUfUmGmUmUmAmUmUmCfAmCfA |
| | 160 | mAmUmCmGmA*mC*mA |
| Duplex ID | SEQ ID NO: | mA*mU*mUmAmCmGfGmUfCfAfCmUmAmUmUmUm |
| NO: 61 | 123 | UmGmG*mU*mA |
| | SEQ ID NO: | PmU*fA*mCmCmAfAmAmAmUmAmGmUmGfAmCfC |
| | 161 | mGmUmAmAmU*mA*mA |
| Duplex ID | SEQ ID NO: 96 | mU*mG*mUmCmAmGfCmAfGfAfAmUmAmCmUmUm |
| NO: 62 | | AmGmA*mU*mA |
| | SEQ ID NO: | PmU*fA*mUmCmUmAmAmGmUmAmUmUmCfUmGf |
| | 162 | CmUmGmAmCmA*mA*mG |
| Duplex ID | SEQ ID NO: 96 | mU*mG*mUmCmAmGfCmAfGfAfAmUmAmCmUmUm |
| NO: 63 | | AmGmA*mU*mA |
| | SEQ ID NO: | PmU*fA*mUmCfUmAfAmGmUmAmUmUmCfUmGfCm |
| | 163 | UmGmAmCmA*mA*mG |
| Duplex ID | SEQ ID NO: 96 | mU*mG*mUmCmAmGfCmAfGfAfAmUmAmCmUmUm |
| NO: 64 | | AmGmA*mU*mA |
| | SEQ ID NO: | PmU*fA*fUmCmUmAfAmGmUmAmUmUmCfUmGfCm |
| | 164 | UmGmAmCmA*mA*mG |
| Duplex ID | SEQ ID NO: 96 | mU*mG*mUmCmAmGfCmAfGfAfAmUmAmCmUmUm |
| NO: 65 | | AmGmA*mU*mA |
| | SEQ ID NO: | PmU*fA*mUmCfUmAmAfGmUmAmUmUmCfUmGfCm |
| | 165 | UmGmAmCmA*mA*mG |
| Duplex ID | SEQ ID NO: 96 | mU*mG*mUmCmAmGfCmAfGfAfAmUmAmCmUmUm |
| NO: 66 | | AmGmA*mU*mA |
| | SEQ ID NO: | PmU*fA*fUmCmUmAmAfGmUmAmUmUmCfUmGfCm |
| | 166 | UmGmAmCmA*mA*mG |
| Duplex ID | SEQ ID NO: | mU*mG*mUmCmAmGmCmAfGfAfAmUmAmCmUmU |
| NO: 67 | 124 | mAmGmA*mU*mA |
| | SEQ ID NO: | PmU*fA*mUmCmUmAmAmGmUmAmUmUmCfUmGf |
| | 162 | CmUmGmAmCmA*mA*mG |
| Duplex ID | SEQ ID NO: | mU*mG*mUmCmAmGmCmAfGfAfAmUmAmCmUmU |
| NO: 68 | 124 | mAmGmA*mU*mA |
| | SEQ ID NO: | PmU*fA*mUmCfUmAfAmGmUmAmUmUmCfUmGfCm |
| | 163 | UmGmAmCmA*mA*mG |
| Duplex ID | SEQ ID NO: | mU*mG*mUmCmAmGmCmAfGfAfAmUmAmCmUmU |
| NO: 69 | 124 | mAmGmA*mU*mA |
| | SEQ ID NO: | PmU*fA*fUmCmUmAfAmGmUmAmUmUmCfUmGfCm |
| | 164 | UmGmAmCmA*mA*mG |
| Duplex ID | SEQ ID NO: | mU*mG*mUmCmAmGmCmAfGfAfAmUmAmCmUmU |
| NO: 70 | 124 | mAmGmA*mU*mA |
| | SEQ ID NO: | PmU*fA*mUmCfUmAmAfGmUmAmUmUmCfUmGfCm |
| | 165 | UmGmAmCmA*mA*mG |
| Duplex ID | SEQ ID NO: | mU*mG*mUmCmAmGmCmAfGfAfAmUmAmCmUmU |
| NO: 71 | 124 | mAmGmA*mU*mA |
| | SEQ ID NO: | PmU*fA*fUmCmUmAmAfGmUmAmUmUmCfUmGfCm |
| | 166 | UmGmAmCmA*mA*mG |
| Duplex ID | SEQ ID NO: | mU*mG*mUmCmAmGmCmAfGfAfAmUmAmCmUmU |
| NO: 72 | 124 | mAmGmA*mU*mA |
| | SEQ ID NO: | PmU*fA*mUmCmUfAmAmGmUmAmUmUmCfUmGfC |
| | 134 | mUmGmAmCmA*mA*mG |
| Duplex ID | SEQ ID NO: | mU*mG*mUmCmAmGfCmAfG-Ab- |
| NO: 73 | 125 | fAmUmAmCmUmUmAmGmA*mU*mA |
| | SEQ ID NO: | PmU*fA*mUmCmUmAmAmGmUmAmUmUmCfUmGf |
| | 162 | CmUmGmAmCmA*mA*mG |
| Duplex ID | SEQ ID NO: | mU*mG*mUmCmAmGfCmAfG-Ab- |
| NO: 74 | 125 | fAmUmAmCmUmUmAmGmA*mU*mA |
| | SEQ ID NO: | PmU*fA*mUmCfUmAfAmGmUmAmUmUmCfUmGfCm |
| | 163 | UmGmAmCmA*mA*mG |
| Duplex ID | SEQ ID NO: | mU*mG*mUmCmAmGfCmAfG-Ab- |
| NO: 75 | 125 | fAmUmAmCmUmUmAmGmA*mU*mA |
| | SEQ ID NO: | PmU*fA*fUmCmUmAfAmGmUmAmUmUmCfUmGfCm |
| | 164 | UmGmAmCmA*mA*mG |
| Duplex ID | SEQ ID NO: | mU*mG*mUmCmAmGfCmAfG-Ab- |
| NO: 76 | 125 | fAmUmAmCmUmUmAmGmA*mU*mA |
| | SEQ ID NO: | PmU*fA*mUmCfUmAmAfGmUmAmUmUmCfUmGfCm |
| | 165 | UmGmAmCmA*mA*mG |
| Duplex ID | SEQ ID NO: | mU*mG*mUmCmAmGfCmAfG-Ab- |
| NO: 77 | 125 | fAmUmAmCmUmUmAmGmA*mU*mA |
| | SEQ ID NO: | PmU*fA*fUmCmUmAmAfGmUmAmUmUmCfUmGfCm |
| | 166 | UmGmAmCmA*mA*mG |
| Duplex ID | SEQ ID NO: | mU*mG*mUmCmAmGfCmAfG-Ab- |
| NO: 78 | 125 | fAmUmAmCmUmUmAmGmA*mU*mA |
| | SEQ ID NO: | PmU*fA*mUmCmUfAmAmGmUmAmUmUmCfUmGfC |
| | 134 | mUmGmAmCmA*mA*mG |
| Duplex ID | SEQ ID NO: | mU*mG*mUmCmAmGmCmAfG-Ab- |
| NO: 79 | 126 | fAmUmAmCmUmUmAmGmA*mU*mA |
| | SEQ ID NO: | PmU*fA*mUmCmUmAmAmGmUmAmUmUmCfUmGf |
| | 162 | CmUmGmAmCmA*mA*mG |
| Duplex ID | SEQ ID NO: | mU*mG*mUmCmAmGmCmAfG-Ab- |
| NO: 80 | 126 | fAmUmAmCmUmUmAmGmA*mU*mA |
| | SEQ ID NO: | PmU*fA*mUmCfUmAfAmGmUmAmUmUmCfUmGfCm |
| | 163 | UmGmAmCmA*mA*mG |
| Duplex ID | SEQ ID NO: | mU*mG*mUmCmAmGmCmAfG-Ab- |
| NO: 81 | 126 | fAmUmAmCmUmUmAmGmA*mU*mA |
| | SEQ ID NO: | PmU*fA*fUmCmUmAfAmGmUmAmUmUmCfUmGfCm |
| | 164 | UmGmAmCmA*mA*mG |
| Duplex ID | SEQ ID NO: | mU*mG*mUmCmAmGmCmAfG-Ab- |
| NO: 82 | 126 | fAmUmAmCmUmUmAmGmA*mU*mA |
| | SEQ ID NO: | PmU*fA*mUmCfUmAmAfGmUmAmUmUmCfUmGfCm |
| | 165 | UmGmAmCmA*mA*mG |
| Duplex ID | SEQ ID NO: | mU*mG*mUmCmAmGmCmAfG-Ab- |
| NO: 83 | 126 | fAmUmAmCmUmUmAmGmA*mU*mA |
| | SEQ ID NO: | PmU*fA*fUmCmUmAmAfGmUmAmUmUmCfUmGfCm |
| | 166 | UmGmAmCmA*mA*mG |
| Duplex ID | SEQ ID NO: | mU*mG*mUmCmAmGmCmAfG-Ab- |
| NO: 84 | 126 | fAmUmAmCmUmUmAmGmA*mU*mA |
| | SEQ ID NO: | PmU*fA*mUmCmUfAmAmGmUmAmUmUmCfUmGfC |
| | 134 | mUmGmAmCmA*mA*mG |
| Duplex ID | SEQ ID NO: | mU*mU*mUmGmUmCmUmAfAfAfUmGmAmGmUmU |
| NO: 85 | 127 | mCmAmC*mG*mA |
| | SEQ ID NO: | PmU*fC*mGmUfGmAfAmCmUmCmAmUmUfUmAfGm |
| | 167 | AmCmAmAmA*mA*mU |
| Duplex ID | SEQ ID NO: | mU*mU*mUmGmUmCmUmAfAfAfUmGmAmGmUmU |
| NO: 86 | 127 | mCmAmC*mG*mA |
| | SEQ ID NO: | PmU*fC*fGmUmGmAfAmCmUmCmAmUmUfUmAfGm |
| | 168 | AmCmAmAmA*mA*mU |
| Duplex ID | SEQ ID NO: | mU*mU*mUmGmUmCmUmAfAfAfUmGmAmGmUmU |
| NO: 87 | 127 | mCmAmC*mG*mA |
| | SEQ ID NO: | PmU*fC*mGmUfGmAmAfCmUmCmAmUmUfUmAfGm |
| | 169 | AmCmAmAmA*mA*mU |
| Duplex ID | SEQ ID NO: | mU*mU*mUmAmAmCmUmAfCfAfCmCmAmGmCmU |
| NO: 88 | 128 | mUmUmG*mA*mA |
| | SEQ ID NO: | PmU*fU*mCmAfAmAfGmCmUmGmGmUmGfUmAfG |
| | 170 | mUmUmAmAmA*mA*mU |
| Duplex ID | SEQ ID NO: | mU*mU*mUmAmAmCmUmAfCfAfCmCmAmGmCmU |
| NO: 89 | 128 | mUmUmG*mA*mA |
| | SEQ ID NO: | PmU*fU*fCmAmAmAfGmCmUmGmGmUmGfUmAfG |
| | 171 | mUmUmAmAmA*mA*mU |
| Duplex ID | SEQ ID NO: | mU*mU*mUmAmAmCmUmAfCfAfCmCmAmGmCmU |
| NO: 90 | 128 | mUmUmG*mA*mA |
| | SEQ ID NO: | PmU*fU*mCmAfAmAmGfCmUmGmGmUmGfUmAfG |
| | 172 | mUmUmAmAmA*mA*mU |
| Duplex ID | SEQ ID NO: | mU*mC*mUmUmGmUmCmAfGfCfAmGmAmAmUmA |
| NO: 91 | 129 | mCmUmU*mA*mA |
| | SEQ ID NO: | PmU*fU*mAmAfGmUfAmUmUmCmUmGmCfUmGfA |
| | 173 | mCmAmAmGmA*mA*mA |
| Duplex ID | SEQ ID NO: | mU*mC*mUmUmGmUmCmAfGfCfAmGmAmAmUmA |
| NO: 92 | 129 | mCmUmU*mA*mA |
| | SEQ ID NO: | PmU*fU*fAmAmGmUfAmUmUmCmUmGmCfUmGfA |
| | 174 | mCmAmAmGmA*mA*mA |
| Duplex ID | SEQ ID NO: | mU*mC*mUmUmGmUmCmAfGfCfAmGmAmAmUmA |
| NO: 93 | 129 | mCmUmU*mA*mA |
| | SEQ ID NO: | PmU*fU*mAmAfGmUmAfUmUmCmUmGmCfUmGfA |
| | 175 | mCmAmAmGmA*mA*mA |
| Duplex ID | SEQ ID NO: | mU*mC*mAmUmCmCmUmAfUfGfAmAmCmCmAmA |
| NO: 94 | 130 | mUmAmG*mC*mA |
| | SEQ ID NO: | PmU*fG*mCmUfAmUfUmGmGmUmUmCmAfUmAfG |
| | 176 | mGmAmUmGmA*mU*mU |
| Duplex ID | SEQ ID NO: | mU*mC*mAmUmCmCmUmAfUfGfAmAmCmCmAmA |
| NO: 95 | 130 | mUmAmG*mC*mA |
| | SEQ ID NO: | PmU*fG*fCmUmAmUfUmGmGmUmUmCmAfUmAfG |
| | 177 | mGmAmUmGmA*mU*mU |
| Duplex ID | SEQ ID NO: | mU*mC*mAmUmCmCmUmAfUfGfAmAmCmCmAmA |
| NO: 96 | 130 | mUmAmG*mC*mA |
| | SEQ ID NO: | PmU*fG*mCmUfAmUmUfGmGmUmUmCmAfUmAfG |
| | 178 | mGmAmUmGmA*mU*mU |
| Duplex ID | SEQ ID NO: | mC*mC*mUmUmUmGmAmAfGfCfCmUmUmGmAmU |
| NO: 97 | 131 | mAmAmA*mG*mA |
| | SEQ ID NO: | PmU*fC*mUmUfUmAfUmCmAmAmGmGmCfUmUfCm |
| | 179 | AmAmAmGmG*mU*mG |
| Duplex ID | SEQ ID NO: | mC*mC*mUmUmUmGmAmAfGfCfCmUmUmGmAmU |
| NO: 98 | 131 | mAmAmA*mG*mA |
| | SEQ ID NO: | PmU*fC*fUmUmUmAfUmCmAmAmGmGmCfUmUfCm |
| | 180 | AmAmAmGmG*mU*mG |
| Duplex ID | SEQ ID NO: | mC*mC*mUmUmUmGmAmAfGfCfCmUmUmGmAmU |
| NO: 99 | 131 | mAmAmA*mG*mA |
| | SEQ ID NO: | PmU*fC*mUmUfUmAmUfCmAmAmGmGmCfUmUfCm |
| | 181 | AmAmAmGmG*mU*mG |
| Duplex ID | SEQ ID NO: | mU*mG*mUmCmAmGfCmAfGfAfAmU[Ahd]mCmUmU |
| NO: 168 | 232 | mAmGmA*mU*mA |
| | SEQ ID NO: | VPmU*fA*mUmCmUfAmAmGmUmAmUmUmCfUmGf |
| | 233 | CmUmGmAmCmA*mA*mG |
| Duplex ID | SEQ ID NO: | mU*mG*mUmCmAmGmCmAfGfAfA[ADS-U] |
| NO: 169 | 234 | mAmCmUmUmAmGmA*mU*mA |
| | SEQ ID NO: | VPmU*fA*mUmCfUmAmAfGmUmAmUmUmCfUmGfC |
| | 235 | mUmGmAmCmA*mA*mG |
| Duplex ID | SEQ ID NO: | mU*mG*mUmCmAmGfCmAfGfAfA[Uhd]mAmCmUmU |
| NO: 170 | 236 | mAmGmA*mU*mA |
| | SEQ ID NO: | VPmU*fA*mUmCmUfAmAmGmUmAmUmUmCfUmGf |
| | 233 | CmUmGmAmCmA*mA*mG |
| Duplex ID | SEQ ID NO: | mU*mG*mUmCmAmGfCmAfGfAfAmU[Uhd]mCmUmU |
| NO: 171 | 237 | mAmGmA*mU*mA |
| | SEQ ID NO: | VPmU*fA*mUmCmUfAmAmGmUmAmUmUmCfUmGf |
| | 233 | CmUmGmAmCmA*mA*mG |
| Duplex ID | SEQ ID NO: | mU*mG*mUmCmAmGmCmAfGfAfA[Uhd]mAmCmUm |
| NO: 172 | 238 | UmAmGmA*mU*mA |
| | SEQ ID NO: | VPmU*fA*mUmCfUmAfAmGmUmAmUmUmCfUmGIC |
| | 239 | mUmGmAmCmA*mA*mG |
| Duplex ID | SEQ ID NO: | mU*mG*mUmCmAmGmCmAfGfAfAmU[Ahd]mCmUm |
| NO: 173 | 240 | UmAmGmA*mU*mA |
| | SEQ ID NO: | VPmU*fA*mUmCfUmAfAmGmUmAmUmUmCfUmGfC |
| | 239 | mUmGmAmCmA*mA*mG |
| Duplex ID | SEQ ID NO: | mU*mG*mUmCmAmGmCmAfGfAfA[Uhd]mAmCmUm |
| NO: 174 | 238 | UmAmGmA*mU*mA |
| | SEQ ID NO: | VPmU*fA*mUmCfUmAmAfGmUmAmUmUmCfUmGfC |
| | 235 | mUmGmAmCmA*mA*mG |
| Duplex ID | SEQ ID NO: | mU*mG*mUmCmAmGmCmAfGfAfAmU[Ahd]mCmUm |
| NO: 175 | 240 | UmAmGmA*mU*mA |
| | SEQ ID NO: | VPmU*fA*mUmCfUmAmAfGmUmAmUmUmCfUmGfC |
| | 235 | mUmGmAmCmA*mA*mG |
| Duplex ID | SEQ ID NO: | mU*mG*mUmCmAmGmCmAfGfAfAmU[ADS-U] |
| NO: 176 | 241 | mCmUmUmAmGmA*mU*mA |
| | SEQ ID NO: | VPmU*fA*mUmCfUmAmAfGmUmAmUmUmCfUmGfC |
| | 235 | mUmGmAmCmA*mA*mG |
| Duplex ID | SEQ ID NO: | mU*mG*mUmCmAmGmCmAfGfAfA[Uhd]mAmCmUm |
| NO: 177 | 238 | UmAmGmA*mU*mA |
| | SEQ ID NO: | VPmU*fA*mUmCmUmAmAmGmUmAmUmUmCfUmG |
| | 242 | fCmUmGmAmCmA*mA*mG |
| Duplex ID | SEQ ID NO: | mU*mG*mUmCmAmGfCmAfGfAfAmU[Ahd]mCmUmU |
| NO: 178 | 232 | mAmGmA*mU*mA |
| | SEQ ID NO: | VPmU*fA*mUmCfUmAfAmGmUmAmUmUmCfUmGfC |
| | 239 | mUmGmAmCmA*mA*mG |
| Duplex ID | SEQ ID NO: | mU*mG*mUmCmAmGmCmAfGfAfAmU[Ahd]mCmUm |
| NO: 179 | 240 | UmAmGmA*mU*mA |
| | SEQ ID NO: | VPmU*fA*fUmCmUmAfAmGmUmAmUmUmCfUmGfC |
| | 242 | mUmGmAmCmA*mA*mG |

TABLE 3B
Listing of additional embodiments of SCN10A RNAi agents and
RNAi agents conjugated to a delivery moiety.

| Duplex | | |
|---|---|---|
| ID NO: | SEQ ID NO: | Modified (5′ to 3′) |
| Duplex ID | SEQ ID NO: | mU*mU*mUmAmAmCfUmAfCfAfCmCmAmGmCmUmU |
| NO: 100 | 94 | mUmG*mA*mA |
| | SEQ ID NO: | mU*fU*mCmAmAfAmGmCmUmGmGmUmGfUmAfGmU |
| | 182 | mUmAmAmA*mA*mU |
| Duplex ID | SEQ ID NO: | mU*mC*mUmUmGmUfCmAfGfCfAmGmAmAmUmAmC |
| NO: 101 | 95 | mUmU*mA*mA |
| | SEQ ID NO: | mU*fU*mAmAmGfUmAmUmUmCmUmGmCfUmGfAmC |
| | 183 | mAmAmGmA*mA*mA |
| Duplex ID | SEQ ID NO: | mU*mG*mUmCmAmGfCmAfGfAfAmUmAmCmUmUmA |
| NO: 102 | 96 | mGmA*mU*mA |
| | SEQ ID NO: | mU*fA*mUmCmUfAmAmGmUmAmUmUmCfUmGfCmU |
| | 184 | mGmAmCmA*mA*mG |
| Duplex ID | SEQ ID NO: | mC*mA*mGmUmGmAfCmUfUfCfAmUmCmGmCmUmA |
| NO: 103 | 97 | mAmU*mC*mA |
| | SEQ ID NO: | mU*fG*mAmUmUfAmGmCmGmAmUmGmAfAmGfUmC |
| | 185 | mAmCmUmG*mU*mG |
| Duplex ID | SEQ ID NO: | mG*mA*mGmCmAmCfAmGfUfGfAmCmUmUmCmAmU |
| NO: 104 | 98 | mCmG*mC*mA |
| | SEQ ID NO: | mU*fG*mCmGmAfUmGmAmAmGmUmCmAfCmUfGmU |
| | 186 | mGmCmUmC*mA*mU |
| Duplex ID | SEQ ID NO: | mU*mU*mUmGmUmCfUmAfAfAfUmGmAmGmUmUmC |
| NO: 105 | 99 | mAmC*mG*mA |
| | SEQ ID NO: | mU*fC*mGmUmGfAmAmCmUmCmAmUmUfUmAfGmA |
| | 187 | mCmAmAmA*mA*mU |
| Duplex ID | SEQ ID NO: | mC*mC*mUmUmUmGfAmAfGfCfCmUmUmGmAmUmA |
| NO: 106 | 100 | mAmA*mG*mA |
| | SEQ ID NO: | mU*fC*mUmUmUfAmUmCmAmAmGmGmCfUmUfCmA |
| | 188 | mAmAmGmG*mU*mG |
| Duplex ID | SEQ ID NO: | mU*mC*mAmUmCmCfUmAfUfGfAmAmCmCmAmAmU |
| NO: 107 | 101 | mAmG*mC*mA |
| | SEQ ID NO: | mU*fG*mCmUmAfUmUmGmGmUmUmCmAfUmAfGmG |
| | 189 | mAmUmGmA*mU*mU |
| Duplex ID | SEQ ID NO: | mG*mA*mAmCmUmCfUmUfUfCfAmGmUmGmCmUmG |
| NO: 108 | 102 | mAmC*mA*mA |
| | SEQ ID NO: | mU*fU*mGmUmCfAmGmCmAmCmUmGmAfAmAfGmA |
| | 190 | mGmUmUmC*mA*mA |
| Duplex ID | SEQ ID NO: | mC*mU*mGmAmAmUfCmUfCfUfUmUmGmUmUmGmG |
| NO: 109 | 103 | mGmG*mU*mA |
| | SEQ ID NO: | mU*fA*mCmCmCfCmAmAmCmAmAmAmGfAmGfAmU |
| | 191 | mUmCmAmG*mU*mG |
| Duplex ID | SEQ ID NO: | mA*mC*mAmUmGmAfGmGfAfCfAmUmCmUmAmGmC |
| NO: 110 | 104 | mUmC*mA*mA |
| | SEQ ID NO: | mU*fU*mGmAmGfCmUmAmGmAmUmGmUfCmCfUmC |
| | 192 | mAmUmGmU*mU*mG |
| Duplex ID | SEQ ID NO: | mU*mA*mUmUmAmCfGmGfUfCfAmCmUmAmUmUmU |
| NO: 111 | 105 | mUmG*mG*mA |
| | SEQ ID NO: | mU*fC*mCmAmAfAmAmUmAmGmUmGmAfCmCfGmU |
| | 193 | mAmAmUmA*mA*mA |
| Duplex ID | SEQ ID NO: | mG*mA*mCmAmAmUfUmCfUfCfUmUmUmGmGmGmC |
| NO: 112 | 106 | mUmU*mG*mA |
| | SEQ ID NO: | mU*fC*mAmAmGfCmCmCmAmAmAmGmAfGmAfAmU |
| | 194 | mUmGmUmC*mU*mU |
| Duplex ID | SEQ ID NO: | mG*mG*mCmAmUmGfAmGfCfCfCmUmAmCmCmUmUm |
| NO: 113 | 107 | CmG*mA*mA |
| | SEQ ID NO: | mU*fU*mCmGmAfAmGmGmUmAmGmGmGfCmUfCmA |
| | 195 | mUmGmCmC*mA*mU |
| Duplex ID | SEQ ID NO: | mG*mG*mCmAmAmUfGmAfGfUfGmUmUmGmUmCmA |
| NO: 114 | 108 | mGmU*mA*mA |
| | SEQ ID NO: | mU*fU*mAmCmUfGmAmCmAmAmCmAmCfUmCfAmU |
| | 196 | mUmGmCmC*mC*mU |
| Duplex ID | SEQ ID NO: | mG*mG*mUmUmGmGfCmAfAfGfCmAmGmCmUmCmC |
| NO: 115 | 109 | mUmA*mG*mA |
| | SEQ ID NO: | mU*fC*mUmAmGfGmAmGmCmUmGmCmUfUmGfCmC |
| | 197 | mAmAmCmC*mA*mG |
| Duplex ID | SEQ ID NO: | mC*mC*mUmCmAmCfCmAfUfCfUmUmCmUmGmCmCm |
| NO: 116 | 110 | UmA*mA*mA |
| | SEQ ID NO: | mU*fU*mUmAmGfGmCmAmGmAmAmGmAfUmGfGmU |
| | 198 | mGmAmGmG*mA*mU |
| Duplex ID | SEQ ID NO: | mC*mA*mCmAmGmAfUmGfAfUfGmGmAmGmUmCmU |
| NO: 117 | 111 | mUmU*mC*mA |
| | SEQ ID NO: | mU*fG*mAmAmAfGmAmCmUmCmCmAmUfCmAfUmC |
| | 199 | mUmGmUmG*mA*mC |
| Duplex ID | SEQ ID NO: | mC*mU*mGmGmCmAfUmAfUfGfUmUmGmGmCmAmC |
| NO: 118 | 112 | mAmG*mC*mA |
| | SEQ ID NO: | mU*fG*mCmUmGfUmGmCmCmAmAmCmAfUmAfUmG |
| | 200 | mCmCmAmG*mG*mG |
| Duplex ID | SEQ ID NO: | mU*mG*mAmGmUmGfUmUfGfUfCmAmGmUmAmUmC |
| NO: 119 | 113 | mAmU*mA*mA |
| | SEQ ID NO: | mU*fU*mAmUmGfAmUmAmCmUmGmAmCfAmAfCmA |
| | 201 | mCmUmCmA*mU*mU |
| Duplex ID | SEQ ID NO: | mU*mC*mCmUmAmUfGmAfAfCfCmAmAmUmAmGmC |
| NO: 120 | 114 | mAmA*mC*mA |
| | SEQ ID NO: | mU*fG*mUmUmGfCmUmAmUmUmGmGmUfUmCfAmU |
| | 202 | mAmGmGmA*mU*mG |
| Duplex ID | SEQ ID NO: | mA*mG*mUmGmUmUfGmUfCfAfGmUmAmUmCmAmU |
| NO: 121 | 115 | mAmA*mC*mA |
| | SEQ ID NO: | mU*fG*mUmUmAfUmGmAmUmAmCmUmGfAmCfAmA |
| | 203 | mCmAmCmU*mC*mA |
| Duplex ID | SEQ ID NO: | mG*mA*mAmUmAmAfCmAfAfGfUmCmUmGmAmCmU |
| NO: 122 | 116 | mGmC*mA*mA |
| | SEQ ID NO: | mU*fU*mGmCmAfGmUmCmAmGmAmCmUfUmGfUmU |
| | 204 | mAmUmUmC*mA*mC |
| Duplex ID | SEQ ID NO: | mC*mG*mUmCmAmUfUmAfCfCfCmUmGmGmCmAmUm |
| NO: 123 | 117 | AmU*mG*mA |
| | SEQ ID NO: | mU*fC*mAmUmAfUmGmCmCmAmGmGmGfUmAfAmU |
| | 205 | mGmAmCmG*mC*mU |
| Duplex ID | SEQ ID NO: | mA*mC*mAmCmUmGfAmCfAfGfGmGmUmCmUmUmC |
| NO: 124 | 118 | mAmC*mC*mA |
| | SEQ ID NO: | mU*fG*mGmUmGfAmAmGmAmCmCmCmUfGmUfCmA |
| | 206 | mGmUmGmU*mA*mC |
| Duplex ID | SEQ ID NO: | mU*mG*mAmAmGmCfCmUfUfGfAmUmAmAmAmGmA |
| NO: 125 | 119 | mUmA*mC*mA |
| | SEQ ID NO: | mU*fG*mUmAmUfCmUmUmUmAmUmCmAfAmGfGmC |
| | 207 | mUmUmCmA*mA*mA |
| Duplex ID | SEQ ID NO: | mU*mA*mUmGmAmGfAmGfUfGfUmCmAmCmUmAmG |
| NO: 126 | 120 | mAmG*mG*mA |
| | SEQ ID NO: | mU*fC*mCmUmCfUmAmGmUmGmAmCmAfCmUfCmUm |
| | 208 | CmAmUmA*mG*mG |
| Duplex ID | SEQ ID NO: | mA*mC*mAmUmUmUfCmAfGfCfCmAmCmUmGmUmCm |
| NO: 127 | 121 | AmU*mU*mA |
| | SEQ ID NO: | mU*fA*mAmUmGfAmCmAmGmUmGmGmCfUmGfAmA |
| | 209 | mAmUmGmU*mC*mU |
| Duplex ID | SEQ ID NO: | mU*mC*mGmAmUmUfGmUfGfAfAmUmAmAmCmAmA |
| NO: 128 | 122 | mGmU*mC*mA |
| | SEQ ID NO: | mnU*fG*mAmCmUfUmGmUmUmAmUmUmCfAmCfAmA |
| | 210 | mUmCmGmA*mC*mA |
| Duplex ID | SEQ ID NO: | mA*mU*mUmAmCmGfGmUfCfAfCmUmAmUmUmUmU |
| NO: 129 | 123 | mGmG*mU*mA |
| | SEQ ID NO: | mU*fA*mCmCmAfAmAmAmUmAmGmUmGfAmCfCmG |
| | 211 | mUmAmAmU*mA*mA |
| Duplex ID | SEQ ID NO: | mU*mG*mUmCmAmGfCmAfGfAfAmUmAmCmUmUmA |
| NO: 130 | 96 | mGmA*mU*mA |
| | SEQ ID NO: | mU*fA*mUmCmUmAmAmGmUmAmUmUmCfUmGfCmU |
| | 212 | mGmAmCmA*mA*mG |
| Duplex ID | SEQ ID NO: | mU*mG*mUmCmAmGfCmAfGfAfAmUmAmCmUmUmA |
| NO: 131 | 96 | mGmA*mU*mA |
| | SEQ ID NO: | mU*fA*mUmCfUmAfAmGmUmAmUmUmCfUmGfCmUm |
| | 213 | GmAmCmA*mA*mG |
| Duplex ID | SEQ ID NO: | mU*mG*mUmCmAmGfCmAfGfAfAmUmAmCmUmUmA |
| NO: 132 | 96 | mnGmA*mU*mA |
| | SEQ ID NO: | mU*fA*fUmCmUmAfAmGmUmAmUmUmCfUmGfCmUm |
| | 214 | GmAmCmA*mA*mG |
| Duplex ID | SEQ ID NO: | mU*mG*mUmCmAmGfCmAfGfAfAmUmAmCmUmUmA |
| NO: 133 | 96 | mGmA*mU*mA |
| | SEQ ID NO: | mU*fA*mUmCfUmAmAfGmUmAmUmUmCfUmGfCmUm |
| | 215 | GmAmCmA*mA*mG |
| Duplex ID | SEQ ID NO: | mU*mG*mUmCmAmGfCmAfGfAfAmUmAmCmUmUmA |
| NO: 134 | 96 | mGmA*mU*mA |
| | SEQ ID NO: | mU*fA*fUmCmUmAmAfGmUmAmUmUmCfUmGfCmUm |
| | 216 | GmAmCmA*mA*mG |
| Duplex ID | SEQ ID NO: | mU*mG*mUmCmAmGmCmAfGfAfAmUmAmCmUmUmA |
| NO: 135 | 124 | mGmA*mU*mA |
| | SEQ ID NO: | mU*fA*mUmCmUmAmAmGmUmAmUmUmCfUmGfCmU |
| | 212 | mGmAmCmA*mA*mG |
| Duplex ID | SEQ ID NO: | mU*mG*mUmCmAmGmCmAfGfAfAmUmAmCmUmUmA |
| NO: 136 | 124 | mGmA*mU*mA |
| | SEQ ID NO: | mU*fA*mUmCfUmAfAmGmUmAmUmUmCfUmGfCmUm |
| | 213 | GmAmCmA*mA*mG |
| Duplex ID | SEQ ID NO: | mU*mG*mUmCmAmGmCmAfGfAfAmUmAmCmUmUmA |
| NO: 137 | 124 | mGmA*mU*mA |
| | SEQ ID NO: | mU*fA*fUmCmUmAfAmGmUmAmUmUmCfUmGfCmUm |
| | 214 | GmAmCmA*mA*mG |
| Duplex ID | SEQ ID NO: | mU*mG*mUmCmAmGmCmAfGfAfAmUmAmCmUmUmA |
| NO: 138 | 124 | mGmA*mU*mA |
| | SEQ ID NO: | mU*fA*mUmCfUmAmAfGmUmAmUmUmCfUmGfCmUm |
| | 215 | GmAmCmA*mA*mG |
| Duplex ID | SEQ ID NO: | mU*mG*mUmCmAmGmCmAfGfAfAmUmAmCmUmUmA |
| NO: 139 | 124 | mGmA*mU*mA |
| | SEQ ID NO: | mU*fA*fUmCmUmAmAfGmUmAmUmUmCfUmGfCmUm |
| | 216 | GmAmCmA*mA*mG |
| Duplex ID | SEQ ID NO: | mU*mG*mUmCmAmGmCmAfGfAfAmUmAmCmUmUmA |
| NO: 140 | 124 | mGmA*mU*mA |
| | SEQ ID NO: | mU*fA*mUmCmUfAmAmGmUmAmUmUmCfUmGfCmU |
| | 184 | mGmAmCmA*mA*mG |
| Duplex ID | SEQ ID NO: | mU*mG*mUmCmAmGfCmAfG-Ab- |
| NO: 141 | 125 | fAmUmAmCmUmUmAmGmA*mU*mA |
| | SEQ ID NO: | mU*fA*mUmCmUmAmAmGmUmAmUmUmCfUmGfCmU |
| | 212 | mGmAmCmA*mA*mG |
| Duplex ID | SEQ ID NO: | mU*mG*mUmCmAmGfCmAfG-Ab- |
| NO: 142 | 125 | fAmUmAmCmUmUmAmGmA*mU*mA |
| | SEQ ID NO: | mU*fA*mUmCfUmAfAmGmUmAmUmUmCfUmGfCmUm |
| | 213 | GmAmCmA*mA*mG |
| Duplex ID | SEQ ID NO: | mU*mG*mUmCmAmGfCmAfG-Ab- |
| NO: 143 | 125 | fAmUmAmCmUmUmAmGmA*mU*mA |
| | SEQ ID NO: | mU*fA*fUmCmUmAfAmGmUmAmUmUmCfUmGfCmUm |
| | 214 | GmAmCmA*mA*mG |
| Duplex ID | SEQ ID NO: | mU*mG*mUmCmAmGfCmAfG-Ab- |
| NO: 144 | 125 | fAmUmAmCmUmUmAmGmA*mU*mA |
| | SEQ ID NO: | mU*fA*mUmCfUmAmAfGmUmAmUmUmCfUmGfCmUm |
| | 215 | GmAmCmA*mA*mG |
| Duplex ID | SEQ ID NO: | mU*mG*mUmCmAmGfCmAfG-Ab- |
| NO: 145 | 125 | fAmUmAmCmUmUmAmGmA*mU*mA |
| | SEQ ID NO: | mU*fA*fUmCmUmAmAfGmUmAmUmUmCfUmGfCmUm |
| | 216 | GmAmCmA*mA*mG |
| Duplex ID | SEQ ID NO: | mU*mG*mUmCmAmGfCmAfG-Ab- |
| NO: 146 | 125 | fAmUmAmCmUmUmAmGmA*mU*mA |
| | SEQ ID NO: | mU*fA*mUmCmUfAmAmGmUmAmUmUmCfUmGfCmU |
| | 184 | mGmAmCmA*mA*mG |
| Duplex ID | SEQ ID NO: | mU*mG*mUmCmAmGmCmAfG-Ab- |
| NO: 147 | 126 | fAmUmAmCmUmUmAmGmA*mU*mA |
| | SEQ ID NO: | mU*fA*mUmCmUmAmAmGmUmAmUmUmCfUmGfCmU |
| | 212 | mGmAmCmA*mA*mG |
| Duplex ID | SEQ ID NO: | mU*mG*mUmCmAmGmCmAfG-Ab- |
| NO: 148 | 126 | fAmUmAmCmUmUmAmGmA*mU*mA |
| | SEQ ID NO: | mU*fA*mUmCfUmAfAmGmUmAmUmUmCfUmGfCmUm |
| | 213 | GmAmCmA*mA*mG |
| Duplex ID | SEQ ID NO: | mU*mG*mUmCmAmGmCmAfG-Ab- |
| NO: 149 | 126 | fAmUmAmCmUmUmAmGmA*mU*mA |
| | SEQ ID NO: | mU*fA*fUmCmUmAfAmGmUmAmUmUmCfUmGfCmUm |
| | 214 | GmAmCmA*mA*mG |
| Duplex ID | SEQ ID NO: | mU*mG*mUmCmAmGmCmAfG-Ab- |
| NO: 150 | 126 | fAmUmAmCmUmUmAmGmA*mU*mA |
| | SEQ ID NO: | mU*fA*mUmCfUmAmAfGmUmAmUmUmCfUmGfCmUm |
| | 215 | GmAmCmA*mA*mG |
| Duplex ID | SEQ ID NO: | mU*mG*mUmCmAmGmCmAfG-Ab- |
| NO: 151 | 126 | fAmUmAmCmUmUmAmGmA*mU*mA |
| | SEQ ID NO: | mU*fA*fUmCmUmAmAfGmUmAmUmUmCfUmGfCmUm |
| | 216 | GmAmCmA*mA*mG |
| Duplex ID | SEQ ID NO: | mU*mG*mUmCmAmGmCmAfG-Ab- |
| NO: 152 | 126 | fAmUmAmCmUmUmAmGmA*mU*mA |
| | SEQ ID NO: | mU*fA*mUmCmUfAmAmGmUmAmUmUmCfUmGfCmU |
| | 184 | mGmAmCmA*mA*mG |
| Duplex ID | SEQ ID NO: | mU*mU*mUmGmUmCmUmAfAfAfUmGmAmGmUmUmC |
| NO: 153 | 127 | mAmC*mG*mA |
| | SEQ ID NO: | mU*fC*mGmUfGmAfAmCmUmCmAmUmUfUmAfGmAm |
| | 217 | CmAmAmA*mA*mU |
| Duplex ID | SEQ ID NO: | mU*mU*mUmGmUmCmUmAfAfAfUmGmAmGmUmUmC |
| NO: 154 | 127 | mAmC*mG*mA |
| | SEQ ID NO: | mU*fC*fGmUmGmAfAmCmUmCmAmUmUfUmAfGmAm |
| | 218 | CmAmAmA*mA*mU |
| Duplex ID | SEQ ID NO: | mU*mU*mUmGmUmCmUmAfAfAfUmGmAmGmUmUmC |
| NO: 155 | 127 | mAmC*mG*mA |
| | SEQ ID NO: | mU*fC*mGmUfGmAmAfCmUmCmAmUmUfUmAfGmAm |
| | 219 | CmAmAmA*mA*mU |
| Duplex ID | SEQ ID NO: | mU*mU*mUmAmAmCmUmAfCfAfCmCmAmGmCmUmU |
| NO: 156 | 128 | mUmG*mA*mA |
| | SEQ ID NO: | mU*fU*mCmAfAmAfGmCmUmGmGmUmGfUmAfGmUm |
| | 220 | UmAmAmA*mA*mU |
| Duplex ID | SEQ ID NO: | mU*mU*mUmAmAmCmUmAfCfAfCmCmAmGmCmUmU |
| NO: 157 | 128 | mUmG*mA*mA |
| | SEQ ID NO: | mU*fU*fCmAmAmAfGmCmUmGmGmUmGfUmAfGmUm |
| | 221 | UmAmAmA*mA*mU |
| Duplex ID | SEQ ID NO: | mU*mU*mUmAmAmCmUmAfCfAfCmCmAmGmCmUmU |
| NO: 158 | 128 | mUmG*mA*mA |
| | SEQ ID NO: | inU*fU*mCmAfAmAmGfCmUmGmGmUmGfUmAfGmUm |
| | 222 | UmAmAmA*mA*mU |
| Duplex ID | SEQ ID NO: | mU*mC*mUmUmGmUmCmAfGfCfAmGmAmAmUmAmC |
| NO: 159 | 129 | mUmU*mA*mA |
| | SEQ ID NO: | mU*fU*mAmAfGmUfAmUmUmCmUmGmCfUmGfAmCm |
| | 223 | AmAmGmA*mA*mA |
| Duplex ID | SEQ ID NO: | mU*mC*mUmUmGmUmCmAfGfCfAmGmAmAmUmAmC |
| NO: 160 | 129 | mUmU*mA*mA |
| | SEQ ID NO: | mU*fU*fAmAmGmUfAmUmUmCmUmGmCfUmGfAmCm |
| | 224 | AmAmGmA*mA*mA |
| Duplex ID | SEQ ID NO: | mU*mC*mUmUmGmUmCmAfGfCfAmGmAmAmUmAmC |
| NO: 161 | 129 | mUmU*mA*mA |
| | SEQ ID NO: | mU*fU*mAmAfGmUmAfUmUmCmUmGmCfUmGfAmCm |
| | 225 | AmAmGmA*mA*mA |
| Duplex ID | SEQ ID NO: | mU*mC*mAmUmCmCmUmAfUfGfAmAmCmCmAmAmU |
| NO: 162 | 130 | mAmG*mC*mA |
| | SEQ ID NO: | mU*fG*mCmUfAmUfUmGmGmUmUmCmAfUmAfGmGm |
| | 226 | AmUmGmA*mU*mU |
| Duplex ID | SEQ ID NO: | mU*mC*mAmUmCmCmUmAfUfGfAmAmCmCmAmAmU |
| NO: 163 | 130 | mAmG*mC*mA |
| | SEQ ID NO: | mU*fG*fCmUmAmUfUmGmGmUmUmCmAfUmAfGmGm |
| | 227 | AmUmGmA*mU*mU |
| Duplex ID | SEQ ID NO: | mU*mC*mAmUmCmCmUmAfUfGfAmAmCmCmAmAmU |
| NO: 164 | 130 | mAmG*mnC*mA |
| | SEQ ID NO: | mU*fG*mCmUfAmUmUfGmGmUmUmCmAfUmAfGmGm |
| | 228 | AmUmGmA*mU*mU |
| Duplex ID | SEQ ID NO: | mC*mC*mUmUmUmGmAmAfGfCfCmUmUmGmAmUmA |
| NO: 165 | 131 | mAmA*mG*mA |
| | SEQ ID NO: | mU*fC*mUmUfUmAfUmCmAmAmGinGmCfUmUfCmAm |
| | 229 | AmAmGmG*mU*mG |
| Duplex ID | SEQ ID NO: | mC*mC*mUmUmUmGmAmAfGfCfCmUmUmGmAmUmA |
| NO: 166 | 131 | mAmA*mG*mA |
| | SEQ ID NO: | mU*fC*fUmUmUmAfUmCmAmAmGmGmCfUmUfCmAm |
| | 230 | AmAmGmG*mU*mG |
| Duplex ID | SEQ ID NO: | mC*mC*mUmUmUmGmAmAfGfCfCmUmUmGmAmUmA |
| NO: 167 | 131 | mAmA*mG*mA |
| | SEQ ID NO: | mU*fC*mUmUfUmAmUfCmAmAmGmGmCfUmUfCmAm |
| | 231 | AmAmGmG*mU*mG |
| Duplex ID | SEQ ID NO: | mU*mG*mUmCmAmGfCmAfGfAfAmU[Ahd]mCmUmUm |
| NO: 180 | 232 | AmGmA*mU*mA |
| | SEQ ID NO: | mU*fA*mUmCmUfAmAmGmUmAmUmUmCfUmGfCmU |
| | 184 | mGmAmCmA*mA*mG |
| Duplex ID | SEQ ID NO: | mU*mG*mUmCmAmGmCmAfGfAfA[ADS-U] |
| NO: 181 | 234 | mAmCmUmUmAmGmA*mU*mA |
| | SEQ ID NO: | mU*fA*mUmCfUmAmAfGmUmAmUmUmCfUmGfCmUm |
| | 215 | GmAmCmA*mA*mG |
| Duplex ID | SEQ ID NO: | mU*mG*mUmCmAmGfCmAfGfAfA[Uhd]mAmCmUmUm |
| NO: 182 | 236 | AmGmA*mU*mA |
| | SEQ ID NO: | mU*fA*mUmCmUfAmAmGmUmAmUmUmCfUmGfCmU |
| | 184 | mnGmAmCmA*mA*mG |
| Duplex ID | SEQ ID NO: | mU*mG*mUmCmAmGfCmAfGfAfAmU[Uhd]mCmUmUm |
| NO: 183 | 237 | AmGmA*mU*mA |
| | SEQ ID NO: | mU*fA*mUmCmUfAmAmGmUmAmUmUmCfUmGfCmU |
| | 184 | mGmAmCmA*mA*mG |
| Duplex ID | SEQ ID NO: | mU*mG*mUmCmAmGmCmAfGfAfA[Uhd]mAmCmUmUm |
| NO: 184 | 238 | AmGmA*mU*mA |
| | SEQ ID NO: | mU*fA*mUmCfUmAfAmGmUmAmUmUmCfUmGfCmUm |
| | 213 | GmAmCmA*mA*mG |
| Duplex ID | SEQ ID NO: | mU*mG*mUmCmAmGmCmAfGfAfAmU[Ahd]mCmUmUm |
| NO: 185 | 240 | AmGmA*mU*mA |
| | SEQ ID NO: | mU*fA*mUmCfUmAfAmGmUmAmUmUmCfUmGfCmUm |
| | 213 | GmAmCmA*mA*mG |
| Duplex ID | SEQ ID NO: | mU*mG*mUmCmAmGmCmAfGfAfA[Uhd]mAmCmUmUm |
| NO: 186 | 238 | AmGmA*mU*mA |
| | SEQ ID NO: | mU*fA*mUmCfUmAmAfGmUmAmUmUmCfUmGfCmUm |
| | 215 | GmAmCmA*mA*mG |
| Duplex ID | SEQ ID NO: | mU*mG*mUmCmAmGmCmAfGfAfAmU[Ahd]mCmUmUm |
| NO: 187 | 240 | AmGmA*mU*mA |
| | SEQ ID NO: | mU*fA*mUmCfUmAmAfGmUmAmUmUmCfUmGfCmUm |
| | 215 | GmAmCmA*mA*mG |
| Duplex ID | SEQ ID NO: | mU*mG*mUmCmAmGmCmAfGfAfAmU[ADS-U] |
| NO: 188 | 241 | mCmUmUmAmGmA*mU*mA |
| | SEQ ID NO: | mU*fA*mUmCfUmAmAfGmUmAmUmUmCfUmGfCmUm |
| | 215 | GmAmCmA*mA*mG |
| Duplex ID | SEQ ID NO: | mU*mG*mUmCmAmGmCmAfGfAfA[Uhd]mAmCmUmUm |
| NO: 189 | 238 | AmGmA*mU*mA |
| | SEQ ID NO: | mU*fA*mUmCmUmAmAmGmUmAmUmUmCfUmGfCmU |
| | 212 | mGmAmCmA*mA*mG |
| Duplex ID | SEQ ID NO: | mU*mG*mUmCmAmGfCmAfGfAfAmU[Ahd]mCmUmUm |
| NO: 190 | 232 | AmGmA*mU*mA |
| | SEQ ID NO: | mU*fA*mUmCfUmAfAmGmUmAmUmUmCfUmGfCmUm |
| | 213 | GmAmCmA*mA*mG |
| Duplex ID | SEQ ID NO: | mU*mG*mUmCmAmGmCmAfGfAfAmU[Ahd]mCmUmUm |
| NO: 191 | 240 | AmGmA*mU*mA |
| | SEQ ID NO: | mnU*fA*fUmCmUmAfAmGmUmAmUmUmCfUmGfCmUm |
| | 214 | GmAmCmA*mA*mG |

In Tables 3A and 3B, modifications are noted immediately prior to the corresponding modified nucleotide. The abbreviation “P” stands for a phosphate group, “m” for methylation of the OH group creating a 2′ methoxy modification on the ribose; “f” for a 2′ fluoro modification of the ribose, “*” for a phosphorothioate modification of the phosphodiester bond of the backbone, “Ab” for an abasic residue); “ads” indicates Formula Ia; Ahd or Uhd indicates a delivery moiety which is 2′-O-hexadecyl nucleobase as shown in Table 10. In some embodiments the RNAi agents comprise a “phosphate analog” modification positioned at the 5′ terminal nucleotide of a sense or antisense oligonucleotide in place of a 5′-phosphate. In some embodiments the 5′ modification is a phosphate group (“p”). In some embodiments the modification is 5′-(E)-vinylphosphonate (5′-VP or VP). In some embodiments, the sense strand comprises a delivery moiety conjugated to a nucleotide at position 12 or 13 from the 5′end of the sense strand, wherein in some embodiments, the lipophilic delivery moiety is optionally of Formula I or 2′-O-hexadecyl.

### EXAMPLES

### Example 1: RNAi Agent Design

RNAi agents were designed to comprise an antisense strand complementary to the 18mer regions of SCN10A having the sequence of transcript NM_000718.4 (SEQ ID NO: 1), as listed in Table 1. The sense and antisense RNA oligonucleotides strands are between 18 and 23 nucleotides in length, with optional overhangs of 1, 2, 3, 4 or 5 nucleotides. The nucleotides feature 1-10 fluoro additions at the 2′ position of ribose, and the remaining nucleotides are methylated at the 2′ position of ribose (creating an 2′ methoxy i.e., a 2′ O-methyl modification).

In some embodiments, the sense and/or the antisense strand independently comprise 1, 2, 3, 4 or 5 fluoro additions at the 2′ position of ribose, and the remaining nucleotides are methylated at the 2′ position of ribose. In some embodiments, the antisense strands are optionally phosphorylated at the 5′ end of the strand. In other embodiments, the antisense strands are optionally modified with a vinyl phosphonate group at the 5′ end of the antisense strand. One or more phosphodiester bonds are present at the 5′ and 3′ ends of each strand. Optionally, there is a VPN at the 5′ end of the antisense strand, e.g., instead of a phosphate.

The 18mer sequences that are used to create complementary antisense strands are shown in Table 1. In some embodiments, antisense strands are designed to be 23 nucleotides in length.

The 18mers in Table 1 were used to create duplexes as shown in Table 2.

### Example 2: Knockdown of SCN10A Gene Expression In Vitro

Knockdown of SCN10A expression by RNAi agents was assayed using the following procedure:

Human iPSC-derived sensory neurons (purchased from Anatomic) were plated and each RNAi agents was added directly to an individual well. The RNAi agents derived from the sequences in Table 2 were added at a single concentration of 1 μM (1000 nM), in duplicate. Treated cells were lysed and an analysis of changes in gene expression in RNAi agent treated cells was measured using Cells-to-CT Kits following the manufacturer's protocol (ThermoFisher A35377). Predesigned gene expression assays (supplied as 20× mixtures) were selected from Applied Bio-systems (Foster City, CA, USA). The efficiencies of these assays (ThermoFisher Hs_01045139_ml SCN10A and ThermoFisher Hs99999905_ml GAPDH) were characterized with a dilution series of cDNA. RT-QPCR was performed in MicroAmp Optical 384-well reaction plates using QuantStudio 7 Flex system. The delta-delta CT method of normalizing to the housekeeping gene GAPDH was used to determine relative amounts of gene expression. GraphPad Prism v9.0 was used to determine IC50 with a three parameter logistic fit.

Select siRNA agents having the nucleotide sequences were tested at multiple concentrations: 4000, 1000, 250, 62.5, 15.6, 3.9, 0.98, and 0.24 nM concentrations of teg-cholesterol conjugated siRNA was used.

SCN10A oligonucleotides were conjugated to an lipophilic delivery moiety as follows: duplexes with ID Nos as shown in Table 4 and Table 5 were conjugated with teg-cholesterol. Teg-cholesterol conjugates were evaluated for knockdown in iPSC cells in vitro. The sense and antisense strands of the conjugated RNAi agents are shown in Table 3A.

Results are shown in Table 4 as percent knockdown (% KD) of the SCN10A transcript.

The RNAi agents conjugated as described above are tested in vitro in iPSC cells using the same method. The results of knockdown at a single concentration or various concentration are shown in Table 4 and Table 5 respectively. Table 5 includes data from multiple concentrations to establish IC50, but only 4 μM and IC50 are shown. ND entries correspond to measurements not determined.

TABLE 4
Summary of % KD for RNAi agents from Table 3A conjugated to
teg-cholesterol tested at single concentration in iPSC cells.

| Duplex ID NO: | % KD 1 uM |
|---|---|
| Duplex ID NO: 32 | 59 |
| Duplex ID NO: 33 | 53 |
| Duplex ID NO: 34 | 49 |
| Duplex ID NO: 35 | 45 |
| Duplex ID NO: 36 | 44 |
| Duplex ID NO: 37 | 44 |
| Duplex ID NO: 38 | 40 |
| Duplex ID NO: 39 | 40 |
| Duplex ID NO: 40 | 39 |
| Duplex ID NO: 41 | 38 |
| Duplex ID NO: 42 | 37 |
| Duplex ID NO: 43 | 35 |
| Duplex ID NO: 44 | 35 |
| Duplex ID NO: 45 | 34 |
| Duplex ID NO: 46 | 34 |
| Duplex ID NO: 47 | 34 |
| Duplex ID NO: 48 | 34 |
| Duplex ID NO: 49 | 34 |
| Duplex ID NO: 50 | 34 |
| Duplex ID NO: 51 | 33 |
| Duplex ID NO: 52 | 32 |
| Duplex ID NO: 53 | 32 |
| Duplex ID NO: 54 | 32 |
| Duplex ID NO: 55 | 32 |
| Duplex ID NO: 56 | 31 |
| Duplex ID NO: 57 | 30 |
| Duplex ID NO: 58 | 30 |
| Duplex ID NO: 59 | 30 |
| Duplex ID NO: 60 | 28 |
| Duplex ID NO: 61 | 28 |

TABLE 5
Summary of % KD and IC50 values RNAi agents with duplexes
as listed inTable 3A which are conjugated to teg-
cholesterol and tested in vitro in iPSC cells.

| Duplex ID NO: | % KD 4 uM | IC50 (uM) |
|---|---|---|
| Duplex ID NO: 32 | 66 | 1380 |
| Duplex ID NO: 33 | 56 | 1314 |
| Duplex ID NO: 34 | 82 | 502 |
| Duplex ID NO: 35 | 45 | 721 |
| Duplex ID NO: 36 | 49 | no fit |
| Duplex ID NO: 37 | 70 | 531 |
| Duplex ID NO: 38 | 68 | 608 |
| Duplex ID NO: 39 | 56 | 2900 |
| Duplex ID NO: 40 | 44 | 1388 |
| Duplex ID NO: 41 | 36 | no fit |
| Duplex ID NO: 42 | ND | ND |
| Duplex ID NO: 43 | ND | ND |
| Duplex ID NO: 44 | ND | ND |
| Duplex ID NO: 45 | ND | ND |
| Duplex ID NO: 46 | ND | ND |
| Duplex ID NO: 47 | ND | ND |
| Duplex ID NO: 48 | ND | ND |
| Duplex ID NO: 49 | ND | ND |
| Duplex ID NO: 50 | ND | ND |
| Duplex ID NO: 51 | ND | ND |
| Duplex ID NO: 52 | ND | ND |
| Duplex ID NO: 53 | ND | ND |
| Duplex ID NO: 54 | ND | ND |
| Duplex ID NO: 55 | ND | ND |
| Duplex ID NO: 56 | ND | ND |
| Duplex ID NO: 57 | ND | ND |
| Duplex ID NO: 58 | ND | ND |
| Duplex ID NO: 59 | ND | ND |
| Duplex ID NO: 60 | ND | ND |
| Duplex ID NO: 61 | ND | ND |
| Duplex ID NO: 62 | 66 | 221 |
| Duplex ID NO: 63 | 67 | 403 |
| Duplex ID NO: 64 | 68 | 1767 |
| Duplex ID NO: 65 | 73 | >4000 |
| Duplex ID NO: 66 | 71 | >4000 |
| Duplex ID NO: 67 | 63 | 852 |
| Duplex ID NO: 68 | 63 | 864 |
| Duplex ID NO: 69 | 58 | 851 |
| Duplex ID NO: 70 | 68 | 161 |
| Duplex ID NO: 71 | 68 | 1879 |
| Duplex ID NO: 72 | 64 | 850 |
| Duplex ID NO: 73 | 68 | 748 |
| Duplex ID NO: 74 | 75 | 3132 |
| Duplex ID NO: 75 | 65 | 1006 |
| Duplex ID NO: 76 | 76 | 1155 |
| Duplex ID NO: 77 | 60 | 314 |
| Duplex ID NO: 78 | 58 | 2315 |
| Duplex ID NO: 79 | 53 | 368 |
| Duplex ID NO: 80 | 50 | 2478 |
| Duplex ID NO: 81 | 37 | ND |
| Duplex ID NO: 82 | 46 | ND |
| Duplex ID NO: 83 | 38 | ND |
| Duplex ID NO: 84 | 56 | 2032 |
| Duplex ID NO: 85 | 38 | ND |
| Duplex ID NO: 86 | 36 | ND |
| Duplex ID NO: 87 | 48 | ND |
| Duplex ID NO: 88 | 28 | ND |
| Duplex ID NO: 89 | 40 | ND |
| Duplex ID NO: 90 | 31 | ND |
| Duplex ID NO: 91 | 38 | ND |
| Duplex ID NO: 92 | 46 | ND |
| Duplex ID NO: 93 | 32 | ND |
| Duplex ID NO: 94 | 51 | 2312 |
| Duplex ID NO: 95 | 29 | ND |
| Duplex ID NO: 96 | 49 | ND |
| Duplex ID NO: 97 | 60 | 333 |
| Duplex ID NO: 98 | 61 | 1764 |
| Duplex ID NO: 99 | 53 | 131 |

### Example 3: Knockdown of SCN10A in Cultured Human DRG Cells

Select RNAi agents were further tested in human DRG culture. DRGs were isolated from deceased human donor and plated in the medium containing DMEM/F12 supplemented with 2% horse serum (ThermoFisher Scientific), 30 ng/ml human NGF-B (N-245; Alomone), 30 ng/ml human GDNF (G-240; Alomone), 10 ng/ml human NT3 (N-260; Alomone), Gem21 NeuroPlex (GeminiBio), and penicillin/streptomycin (10378016; ThermoFisher Scientific), followed by 3 day maintenance. Then, siRNAs (conjugated with Teg-cholesterol) were directly added to the DRG neuronal culture at the final concentration of 1000 nM and waited for 5 days before harvest.

RNA was harvested. Specifically, siRNA-treated cells were lysed, and cellular RNA was isolated using RNAdvance v2 for Cell kit (A47943; Beckman Coulter). From 60 ul of eluted RNA, 1.5 μl of total RNA was utilized for RT-qPCR using Luna Universal Probe One-Step RT-QPCR kit (E3006E; NEB) following the manufacturer's instructions. Taqman assays for SCN10A (Hs01045137_ml, Thermo Fisher), GAPDH (Hs99999905_ml, Thermo Fisher), and STMN2 (Hs00975900_ml, Thermo Fisher) were used to measure gene expression using QuanStudio 6 Flex system. Delta-delta Ct of RT-qPCR data was analyzed using Design & Analysis Software 2.6.0 and following data visualization by GraphPad 9.0.

The results are shown in Table 6 as percent knockdown (% KD) of SCN10A transcript levels.

TABLE 6
Summary of % Knockdown with duplexes as listed inTable 3A which
are conjugated to teg-cholesterol in human DRG culture.

| | % KD in human DRG | |
|---|---|---|
| Duplex No. | culture at 1000 nM | N = 4, (STDEV) |
| 32 | 77.2 | (4.0) |
| 33 | 66.5 | (2.8) |
| 34 | 80.3 | (3.2) |
| 37 | 76.1 | (2.3) |
| 38 | 82.8 | (2.6) |

### Example 4: RNAi Agents Administered In Vivo in Rats

Lipid conjugated siRNAs were prepared as described in the art. A 16 carbon carboxylic acid was attached to the nucleotide at the thirteenth position of the sense strand. The 5′ end of the antisense strand was modified with a 5′ VP moiety. In Table 7, Duplex 100 is Duplex 32 including a 16 carbon chain displayed off of a nucleotide of the 2′ hydroxy group of the nucleotide at the thirteenth position of the sense strand and the 5′ end of the antisense strand was modified with a 5′ VP moiety, instead of a 5′P. In Table 7, Duplex 101 is Duplex 33 including a 16 carbon chain displayed off of a nucleotide of the 2′ hydroxy group of the nucleotide at the thirteenth position of the sense strand and the 5′ end of the antisense strand was modified with a 5′ VP moiety, instead of a 5′P. In Table 7, Duplex 102 is Duplex 34 including a 16 carbon chain displayed off of a nucleotide of the 2′ hydroxy group of the nucleotide at the thirteenth position of the sense strand and the 5′ end of the antisense strand was modified with a 5′ VP moiety, instead of a 5′P. In Table 7, Duplex 105 is Duplex 37 including a 16 carbon chain displayed off of a nucleotide of the 2′ hydroxy group of the nucleotide at the thirteenth position of the sense strand and the 5′ end of the antisense strand was modified with a 5′ VP moiety, instead of a 5′P. In Table 7, Duplex 106 is Duplex 38 including a 16 carbon chain displayed off of a nucleotide of the 2′ hydroxy group of the nucleotide at the thirteenth position of the sense strand and the 5′ end of the antisense strand was modified with a 5′ VP moiety, instead of a 5′P. In Table 7, Duplex 107 is Duplex 39 including a 16 carbon chain displayed off of a nucleotide of the 2′ hydroxy group of the nucleotide at the thirteenth position of the sense strand and the 5′ end of the antisense strand was modified with a 5′ VP moiety, instead of a 5′P.

Rats expressing the human SCN10A gene at the rat locus were administered 4 μg of siRNA or vehicle control, intrathecally. Animals were sacrificed 2 weeks later, and the cervical dorsal root ganglia (CDRG), lumbar dorsal root ganglia (LDRG), and high-thoracic dorsal root ganglia (TDRG) were isolated. RNA was prepared as described above, and RT-PCR performed as described above. Percent SCN10A mRNA remaining was measured relative to GAPDH. Results are shown in Table 7.

TABLE 7
Percent SCN10A mRNA remaining in DRG after
administering RNAi agents in vivo.

| | % mRNA | % mRNA | % mRNA |
|---|---|---|---|
| | remaining | remaining | remaining |
| Duplex No. | in CDRG | in LDRG | in TDRG |
| 100 | 78 | 53 | 82 |
| 101 | 75 | 49 | 64 |
| 102 | 70 | 37 | 70 |
| 105 | 82 | 46 | 86 |
| 106 | 65 | 31 | 82 |
| 107 | 89 | 57 | 79 |

Rats expressing the human SCN10A gene at the rat locus were administered 300 ug of siRNA or vehicle control, intrathecally. Animals were sacrificed 1, 2 or 4 weeks later (see Table 8), and the cervical dorsal root ganglia (CDRG), lumbar dorsal root ganglia (LDRG), and high-thoracic dorsal root ganglia (TDRG) were isolated. RNA was prepared as described above, and RT-PCR performed as described above. Percent SCN10A mRNA remaining was measured relative to GAPDH. Results are shown in Table 8.

TABLE 8
Summary of % SCN10A mRNA remaining
in DRGs from in vivo rat studies.

| | % | % | % | % |
|---|---|---|---|---|
| | Remaining | Remaining | Remaining | Remaining |
| Duplex | LDRG 7 day | LDRG 14 day | LDRG 7 day | LDRG 28 day |
| NO: | 300 ug | 300 ug | 300 ug | 300 ug |
| 168 | 37% | 37% | | |
| 171 | | 33% | | |
| 173 | 47% | | | |
| 174 | | | 47% | |
| 175 | 39% | | 40% | 47% |
| 169 | | | 36% | 21% |
| 176 | | | 32% | 21% |
| 178 | 33% | | | |
| 179 | 61% | | | |

### Example 5: RNAi Agents Administered In Vivo in a Non-Human Primate (NHP) Study

In vivo testing of duplex No. 168 in Cynomolgus monkey (Macaca fascicularis) was conducted to assess the efficacy of SCN10A siRNA. In order to elucidate the efficacy of the siRNA in silencing the target gene; n=4/group cynomolgus monkeys were ported with indwelling catheters intrathecally in the lumbar region. The monkeys were infused with either aCSF or duplex No. 168 (8 mg/ml in aCSF) over 15 mins and were perfused 29 days later. Tissues collected at necropsy included dorsal root ganglion, spinal cord, nerves, skin biopsies. qPCR was performed to determine mRNA knockdown in target regions of interest. Table 9 below shows SCN10A mRNA remaining observed in lumbar DRGs, 29 days after a single administration of the siRNA.

TABLE 9
Summary of % mRNA remaining in
DRGs from in vivo NHP study.

| | % remaining | % remaining |
|---|---|---|
| Duplex NO: | LDRG 6 mg | LDRG 20 mg |
| 168 | ~65% | 40% |

### Example 6: Lipophilic Delivery Moieties, Modified Nucleotides Comprising the Same and Nucleic Acids Comprising Modified Nucleotides

Certain abbreviations are defined as follows: “ACN” refers to acetonitrile; “AEX” refers to anion exchange; “C/D” refers to cleavage and deprotection; “CPG” refers to controlled pore glass; “aCSF” refers to artificial cerebral spinal fluid; “DCM” refers to dichloromethane; “DEA” refers to diethylamine; “DIPEA” refers to N,N-diisopropylethylamine; “DMA” refers to dimethylacetamide; “DMAP” refers to 4-dimethylaminopyridine; “DMF” refers to dimethylformamide; “DMSO” refers to dimethyl sulfoxide; “DMT” refers to 4,4′-dimethoxytrityl; “EDCI” refers to 1-ethyl-3-(3-dimethylaminopropyl) carbodiimide; “ES/MS” refers to electrospray mass spectrometry; “EtOAc” refers to ethyl acetate; “EtOH” refers to ethanol and ethyl alcohol; “IP-RP” refers to ion-pair reverse phase; “LC/MS” refers to liquid chromatography-mass spectrometry; “MeOH” refers to methanol and methyl alcohol; “MPA” refers to mobile phase A; “MPB” refers to mobile phase B; “MWCO” refers to molecular weight cut-off; “NaOAc” refers to sodium acetate; “NHS” refers to N-hydroxysuccinimide; “NMR” refers to nuclear magnetic resonance; “PBS” phosphate-buffered saline; “PVDF” refers to polyvinylidene fluoride; “RP” refers to reverse phase; “siRNA” refers to small interfering ribonucleic acid; “TCEP” refers to tris(2-carboxyethyl)phosphine; “TEA” refers to triethylamine; “TFA” refers to trifluoracetic acid; “THF” refers to tetrahydrofuran; “UPLC” refers to ultra-performance liquid chromatography; and “UV” refers to ultraviolet.

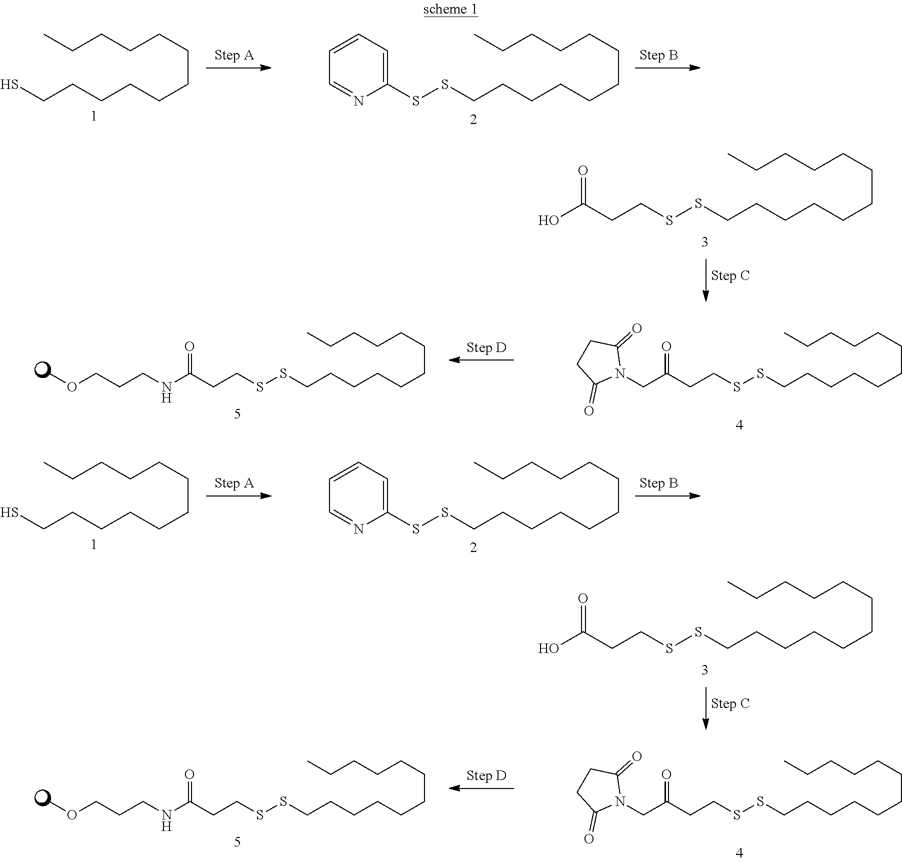

Scheme 1, step A depicts the reaction of compound (1) with 2,2′-dipyridyl disulfide in a solvent system such as MeOH and THF to give compound (2). Step B shows the reaction of compound (2) with 3-sulfanylpropionic acid in a solvent such as MeOH to give compound (3). Step C shows the addition of NHS to compound (3) using a coupling reagent such as EDCI and a catalyst such as DMAP in a solvent such as DCM to give compound (4). Step D shows the addition of compound (4) to an appropriate modified sense strand in the presence of a borate buffer to give compound (5).

### Preparation 1

### 2-(Dodecyldisulfaneyl)pyridine

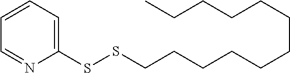 [SMILES: CCCCCCCCCCCCSSc1ccccn1]

1-Dodecanethiol (12.7 g, 61.4 mmol) was added to a solution of 2,2′-dipyridyl disulfide (20.5 g, 92.1 mmol) in MeOH (90 mL) and THF (5 mL). The mixture was stirred at ambient temperature for 16 hours then concentrated in vacuo. The resulting residue was purified via silica gel flash chromatography eluting with 0-15% EtOAc in hexanes to give the title compound as a colorless oil (14.35 g, 75%). ES/MS (m/z): 312(M+H).

### Preparation 2

### 3-(Dodecyldisulfaneyl)propanoic acid

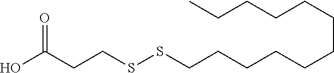 [SMILES: CCCCCCCCCCCCSSCCC(=O)O]

3-Sulfanylpropionic acid (7.58 g, 71.44 mmol) was added to a solution of 2-(dodecyldisulfaneyl)pyridine (18.55 g, 59.5 mmol) in MeOH (60 mL). The reaction was stirred at ambient temperature for 1 hour, then concentrated in vacuo. The resulting residue was purified via silica gel flash chromatography eluting with 5-30% EtO Ac in hexanes to give the title compound as a colorless oil (14 g, 76%). ¹H NMR (DMSO-d6) δ 2.86 (t, 2H, J=7.0 Hz), 2.71 (t, 2H, J=7.0 Hz), 2.62 (t, 2H, J=7.0 Hz), 1.61 (quint, 2H), 1.33 (q, 2H), 1.28 (s, 16H), 0.90 (t, 3H, J=6.8 Hz).

### Preparation 3

### 2,5-Dioxopyrrolidin-1-yl 3-(dodecyldisulfaneyl)propanoate

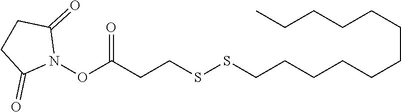 [SMILES: CCCCCCCCCCCCSSCCC(=O)ON1C(=O)CCC1=O]

NHS (1.35 g, 11.7 mmol) was added to a solution of 3-(dodecyldisulfaneyl)propanoic acid (3.0 g, 9.8 mmol), EDCI (2.25 g, 11.7 mmol), and DMAP (0.24 g, 2 mmol) in DCM (39 mL). The mixture was stirred at ambient temperature for 3 hours, then concentrated in vacuo. The resulting residue was purified via silica gel flash chromatography eluting with 0-40% EtOAc in hexanes to give the title compound as a white solid (3.2 g, 81%). ¹H NMR (DMSO-d6) δ 3.10 (t, 2H, J=6.3 Hz), 2.99 (t, 2H, J=6.3 Hz), 2.80 (s, 4H), 2.75 (t, 2H, J=7.0 Hz), 1.61 (quint, 2H), 1.33 (q, 2H), 1.28 (s, 16H), 0.90 (t, 3H, J=6.8 Hz).

C12 ADS Linked siRNA

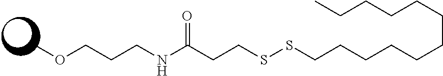 [SMILES: CCCCCCCCCCCCSSCCC(=O)NCCCOC]

A sense strand synthesized using conditions found in the protocols below (3.1 g, 0.44 mmol) in 4× borate buffer water (113 mL) was treated with a solution of 2,5-dioxopyrrolidin-1-yl 3-(dodecyldisulfaneyl)propanoate (5.3 g, 4.4 mmol) in ACN (113 mL). The solution was shaken for 1.5 hours at 30° C. The reaction was quenched by diluting with water and adjusting the pH=7 with 1.2M aqueous HCl. The solution was then concentrated via Genevac to remove the organic solvent and afford the crude oligonuleotides.

The crude oligonucleotides were purified via AKTA™ Pure purification system using reverse phase on a source 15RPC column (MPA: 50 mM NaOAc with 10% ACN and MPB: 80% ACN/water). In all cases, fractions which contained a mass purity greater than 85% without impurities >5% were combined.

The purified oligonucleotides were desalted using 15 mL 3K MWCO centrifugal spin tubes at 3500×g for ~30 minutes. The oligonucleotides were rinsed with RNAse free water until the eluent conductivity reached <100 usemi/cm. After desalting was complete, 2-3 mL of RNAse free water was added then aspirated 10× and the retainment was transferred to a 50 mL falcon tube. This was repeated until complete transfer of oligo by measuring concentration of compound on filter via nanodrop. The final oligonucleotide was then nano filtered 2× via 15 mL 100K MWCO centrifugal spin tubes at 3500×g for 2 min. The final desalted oligonucleotides were analyzed for concentration (nano drop at A260), characterized by IP-RP, LCMS for mass purity, and UPLC for UV-purity. ES/MS (m/z): 7324.6(M+H).

TABLE 10
Non-limiting embodiments of delivery moieties.

| Delivery | |
|---|---|
| Moiety | Structure |
| 1 | Uhd (2′-O-hexadecyl uridine) |
| | 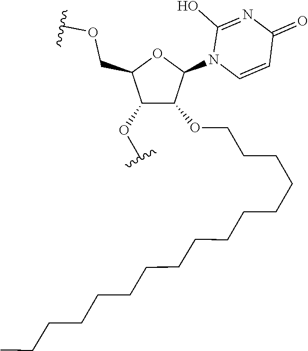 [SMILES: *OC[C@H]1O[C@@H](n2ccc(=O)nc2O)[C@H](OCCCCCCCCCCCCCCCC)[C@@H]1O*] |
| 2 | Ahd (2′-O-hexadecyl adenosine) |
| | 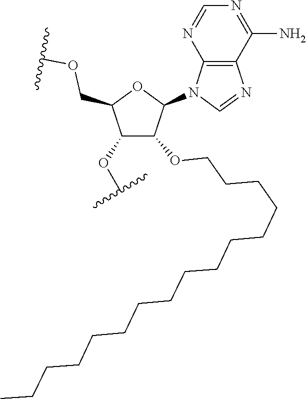 [SMILES: *OC[C@H]1O[C@@H](n2cnc3c(N)ncnc32)[C@H](OCCCCCCCCCCCCCCCC)[C@@H]1O*] |
| 3 | Chd (2′-O-hexadecyl cytidine) |
| | 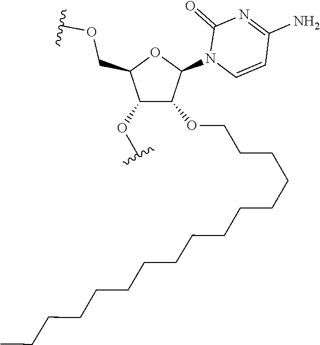 [SMILES: *OC[C@H]1O[C@@H](n2ccc(N)nc2=O)[C@H](OCCCCCCCCCCCCCCCC)[C@@H]1O*] |
| 4 | Ghd (2′-O-hexadecyl guanine) |
| | 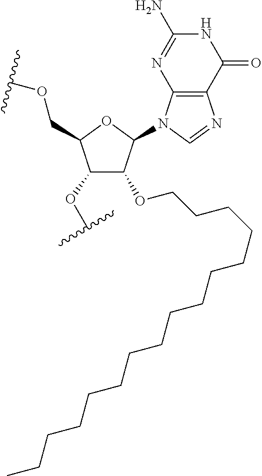 [SMILES: *OC[C@H]1O[C@@H](n2cnc3c(=O)[nH]c(N)nc32)[C@H](OCCCCCCCCCCCCCCCC)[C@@H]1O*] |

Delivery moieties in Table 10 are synthesized and conjugated by standard methods. See e.g. WO2019/217459.

### Example 7: Tissue Distribution and Microgliosis Analyses

Selected SCN10A RNAi agents are evaluated in rats for their tissues distribution and microgliosis. Rats receive intrathecal injection of the RNAi agent of Duplex 168 or Duplex 169 or PBS (phosphate buffered saline). Animals are sacrificed 2 months after the injection.

### RNAi Agent Tissue Distribution and Microgliosis Analysis

Selected tissues from the brain or the spinal cord of the treated rats are prepared for imaging. Images of rat tissues are analyzed for inflammation based on the criteria in Table 11.

TABLE 11
Microgliosis Scoring

| Microgliosis Scoring | Description |
|---|---|
| No Inflammation | No activated microglia. |
| Minimal Inflammation | 3-5 focal lesions of activated microglia across the tissue. |
| | Microglia primary processes thicken, and cell bodies |
| | begins to round. Microglia upregulate the marker IBA1. |
| Mild Inflammation | 5-10 focal lesions of activated microglia across the |
| | tissue. Microglia primary process have thickened and |
| | begin to retract into the cell body. Microglia have lost |
| | most secondary and tertiary processes. The number of |
| | microglia increases, and they continue to upregulate the |
| | marker IBA1. |
| Moderate Inflammation | Diffuse activated microglia across the tissue. Microglia |
| | have become ameboid in shape. There may be small |
| | primary process, but no secondary, or tertiary processes. |
| | Microglia numbers continue to increase, and they |
| | continue to upregulate IBA1 marker expression. |
| Severe Inflammation | Widespread, diffuse activated microglia across the |
| | tissue. Microglia are ameboid with no processes. |
| | Microglia numbers have increased and there is high |
| | expression of the marker IBA1. |

1-7. (canceled)

8. A SCN10A RNA interference (RNAi) agent comprising a sense strand and an antisense strand, wherein the sense strand and the antisense strand form a duplex, and wherein:
a) the sense strand comprises SEQ ID NO: 33 and the antisense strand comprises SEQ ID NO: 64;
b) the sense strand comprises SEQ ID NO: 34 and the antisense strand comprises SEQ ID NO: 65;
c) the sense strand comprises SEQ ID NO: 35 and the antisense strand comprises SEQ ID NO: 66;
d) the sense strand comprises SEQ ID NO: 36 and the antisense strand comprises SEQ ID NO: 67;
e) the sense strand comprises SEQ ID NO: 37 and the antisense strand comprises SEQ ID NO: 68;
f) the sense strand comprises SEQ ID NO: 38 and the antisense strand comprises SEQ ID NO: 69;
g) the sense strand comprises SEQ ID NO: 39 and the antisense strand comprises SEQ ID NO: 70;
h) the sense strand comprises SEQ ID NO: 40 and the antisense strand comprises SEQ ID NO: 71;
i) the sense strand comprises SEQ ID NO: 41 and the antisense strand comprises SEQ ID NO: 72;
j) the sense strand comprises SEQ ID NO: 42 and the antisense strand comprises SEQ ID NO: 73;
k) the sense strand comprises SEQ ID NO: 43 and the antisense strand comprises SEQ ID NO: 74;
l) the sense strand comprises SEQ ID NO: 44 and the antisense strand comprises SEQ ID NO: 75;
m) the sense strand comprises SEQ ID NO: 45 and the antisense strand comprises SEQ ID NO: 76;
n) the sense strand comprises SEQ ID NO: 46 and the antisense strand comprises SEQ ID NO: 77;
o) the sense strand comprises SEQ ID NO: 47 and the antisense strand comprises SEQ ID NO: 78;
p) the sense strand comprises SEQ ID NO: 48 and the antisense strand comprises SEQ ID NO: 79;
q) the sense strand comprises SEQ ID NO: 49 and the antisense strand comprises SEQ ID NO: 80;
r) the sense strand comprises SEQ ID NO: 50 and the antisense strand comprises SEQ ID NO: 81;
s) the sense strand comprises SEQ ID NO: 51 and the antisense strand comprises SEQ ID NO: 82;
t) the sense strand comprises SEQ ID NO: 52 and the antisense strand comprises SEQ ID NO: 83;
u) the sense strand comprises SEQ ID NO: 53 and the antisense strand comprises SEQ ID NO: 84;
v) the sense strand comprises SEQ ID NO: 54 and the antisense strand comprises SEQ ID NO: 85;
w) the sense strand comprises SEQ ID NO: 55 and the antisense strand comprises SEQ ID NO: 86;
x) the sense strand comprises SEQ ID NO: 56 and the antisense strand comprises SEQ ID NO: 87;
y) the sense strand comprises SEQ ID NO: 57 and the antisense strand comprises SEQ ID NO: 88;
z) the sense strand comprises SEQ ID NO: 58 and the antisense strand comprises SEQ ID NO: 89;
aa) the sense strand comprises SEQ ID NO: 59 and the antisense strand comprises SEQ ID NO: 90;
bb) the sense strand comprises SEQ ID NO: 60 and the antisense strand comprises SEQ ID NO: 91;
cc) the sense strand comprises SEQ ID NO: 61 and the antisense strand comprises SEQ ID NO: 92;
dd) the sense strand comprises SEQ ID NO: 62 and the antisense strand comprises SEQ ID NO: 93; or
ee) the sense strand comprises SEQ ID NO: 63 and the antisense strand comprises SEQ ID NO: 66;
wherein the sense or antisense strand each independently comprise one or more modified nucleotides, and wherein one or more internucleotide linkages of the sense strand or the antisense strand are modified internucleotide linkages-comprising a pair of nucleic acid sequences as listed in Table 2.

9. The RNAi agent of claim 8, wherein the sense strand comprises SEQ ID NO: 35; and the antisense strand comprises SEQ ID NO:66, or the sense strand comprises SEQ ID NO: 39 and the antisense strand comprises SEQ ID NO: 70.

10. The RNAi agent of claim 8, wherein the RNAi agent further comprises a delivery moiety.

11-12. (canceled)

13. The RNAi agent of claim 8, wherein the one or more modified nucleotides are independently a 2′-fluoro modified nucleotide or a 2′-O-methyl modified nucleotide.

14. The RNAi agent of claim 13, wherein each nucleotide of the sense and each nucleotide of the antisense strand is modified.

15. The RNAi agent of claim 8, wherein the antisense strand is 23 nucleotides in length and wherein each nucleotide of the antisense strand is a modified nucleotide, and wherein one or more of the modified nucleotides comprises a 2′ fluoro modification that is present at a group of positions as follows:
a. Positions 2, 3, 7, 14, and 16 from the 5′ end of the antisense strand;
b. Positions 2, 5, 7, 14, and 16 from the 5′ end of the antisense strand;
c. Positions 2, 3, 8, 14, and 16 from the 5′ end of the antisense strand;
d. Positions 2, 5, 8, 14, and 16 from the 5′ end of the antisense strand;
e. Positions 2, 14, and 16, from the 5′ end of the antisense strand, or
f. Positions 2, 6, 14, and 16 from the 5′ end of the antisense strand.

16. The RNAi agent of claim 8, wherein the sense strand is 21 nucleotides in length and wherein each nucleotide of the sense strand is a modified nucleotide, and wherein one or more of the modified nucleotides comprises a 2′ fluoro modification that is present at a group of positions as follows:
a. positions 9, 10, and 11 from the 5′-end of the sense strand;
b. positions 7, 9, and 11 from the 5′-end of the sense strand;
c. positions 7, 9, and 10 from the 5′-end of the sense strand;
d. positions 7, 10, and 11 from the 5′-end of the sense strand, or
e. positions 7, 9, 10, and 11 from the 5′ end of the sense strand.

17. The RNAi agent of claim 8, wherein the RNAi agent comprises one or more modified internucleotide linkages that are phosphorothioate linkages.

18. The RNAi agent of claim 17, wherein the sense strand has four or five phosphorothioate linkages.

19. The RNAi agent of claim 18, wherein the antisense strand has four or five phosphorothioate linkages.

20. The RNAi agent of claim 8, wherein the nucleotide at the 5′ end of the antisense strand has a phosphate group or a phosphate analog.

21. The RNAi agent of claim 8, wherein the nucleotide at the 5′ end of the antisense strand has a phosphate analog that is 5′-(E)-vinylphosphonate.

22. The RNAi agent of claim 8, wherein the sense strand has a modification or further modification that is an abasic moiety.

23. The RNAi agent of claim 8, wherein each nucleotide at positions 2, 6, 14, and 16 from the 5′ end of the antisense strand and positions 7, 9, 10, and 11 from the 5′ end of the sense strand comprises a 2′ fluoro modification.

24. The RNAi agent of claim 8, wherein each nucleotide at positions 2, 5, 7, 14, and 16 from the 5′ end of the antisense strand and positions 9, 10, and 11 from the 5′ end of the sense strand comprises a 2′ fluoro modification.

25. The RNAi agent of claim 8, wherein each nucleotide at positions 2, 3, 7, 14, and 16 from the 5′ end of the antisense strand and positions 9, 10, and 11 from the 5′ end of the sense strand comprises a 2′ fluoro modification.

26. The RNAi agent of claim 8, wherein each nucleotide at positions 2, 5, 8, 14, and 16 from the 5′ end of the antisense strand and positions 9, 10, and 11 from the 5′ end of the sense strand comprises a 2′ fluoro modification.

27. The RNAi agent of claim 8, wherein each nucleotide at positions 2, 14, and 16 from the 5′ end of the antisense strand and positions 9, 10, and 11 from the 5′ end of the sense strand comprises a 2′ fluoro modification.

28. The RNAi agent of claim 8, wherein each nucleotide at positions 2, 5, 7, 14, and 16 from the 5′ end of the antisense strand and positions 7, 9, 10, and 11 from the 5′ end of the sense strand comprises a 2′ fluoro modification.

29-32. (canceled)

33. The RNAi agent of claim 8, wherein:
a. the sense strand comprises SEQ ID NO: 94 and the antisense strand comprises SEQ ID NO: 132;
b. the sense strand comprises SEQ ID NO: 95 and the antisense strand comprises SEQ ID NO: 133;
c. the sense strand comprises SEQ ID NO: 96 and the antisense strand comprises SEQ ID NO: 134;
d. the sense strand comprises SEQ ID NO: 97 and the antisense strand comprises SEQ ID NO: 135;
e. the sense strand comprises SEQ ID NO: 98 and the antisense strand comprises SEQ ID NO: 136;
f. the sense strand comprises SEQ ID NO: 99 and the antisense strand comprises SEQ ID NO: 137;
g. the sense strand comprises SEQ ID NO: 100 and the antisense strand comprises SEQ ID NO: 138;
h. the sense strand comprises SEQ ID NO: 101 the antisense strand comprises SEQ ID NO: 139;
i. the sense strand comprises SEQ ID NO: 102 and the antisense strand comprises SEQ ID NO: 140;
j. the sense strand comprises SEQ ID NO: 103 and the antisense strand comprises SEQ ID NO: 141;
k. the sense strand comprises SEQ ID NO: 104 and the antisense strand comprises SEQ ID NO: 142;
l. the sense strand comprises SEQ ID NO: 105 and the antisense strand comprises SEQ ID NO: 143;
m. the sense strand comprises SEQ ID NO: 106 and the antisense strand comprises SEQ ID NO: 144;
n. the sense strand comprises SEQ ID NO: 107 and the antisense strand comprises SEQ ID NO: 145;
o. the sense strand comprises SEQ ID NO: 108 and the antisense strand comprises SEQ ID NO: 146;
p. the sense strand comprises SEQ ID NO: 109 and the antisense strand comprises SEQ ID NO: 147;
q. the sense strand comprises SEQ ID NO: 110 and the antisense strand comprises SEQ ID NO: 148;
r. the sense strand comprises SEQ ID NO: 111 and the antisense strand comprises SEQ ID NO: 149;
s. the sense strand comprises SEQ ID NO: 112 and the antisense strand comprises SEQ ID NO: 150;
t. the sense strand comprises SEQ ID NO: 113 and the antisense strand comprises SEQ ID NO: 151;
u. the sense strand comprises SEQ ID NO: 114 and the antisense strand comprises SEQ ID NO: 152;
v. the sense strand comprises SEQ ID NO: 115 and the antisense strand comprises SEQ ID NO: 153;
w. the sense strand comprises SEQ ID NO: 116 and the antisense strand comprises SEQ ID NO: 154;
x. the sense strand comprises SEQ ID NO: 117 and the antisense strand comprises SEQ ID NO: 155;
y. the sense strand comprises SEQ ID NO: 118 and the antisense strand comprises SEQ ID NO: 156;
z. the sense strand comprises SEQ ID NO: 119 and the antisense strand comprises SEQ ID NO: 157;
aa. the sense strand comprises SEQ ID NO: 120 and the antisense strand comprises SEQ ID NO: 158;
bb. the sense strand comprises SEQ ID NO: 121 and the antisense strand comprises SEQ ID NO: 159;
cc. the sense strand comprises SEQ ID NO: 122 and the antisense strand comprises SEQ ID NO: 160;
dd. the sense strand comprises SEQ ID NO: 123 and the antisense strand comprises SEQ ID NO: 161;
ee. the sense strand comprises SEQ ID NO: 96 and the antisense strand comprises SEQ ID NO: 162;
ff. the sense strand comprises SEQ ID NO: 96 and the antisense strand comprises SEQ ID NO: 163;
gg. the sense strand comprises SEQ ID NO: 96 and the antisense strand comprises SEQ ID NO: 164;
hh. the sense strand comprises SEQ ID NO: 96 and the antisense strand comprises SEQ ID NO: 165;
ii. the sense strand comprises SEQ ID NO: 96 and the antisense strand comprises SEQ ID NO: 166;
jj. the sense strand comprises SEQ ID NO: 124 and the antisense strand comprises SEQ ID NO: 162;
kk. the sense strand comprises SEQ ID NO: 124 and the antisense strand comprises SEQ ID NO: 163;
ll. the sense strand comprises SEQ ID NO: 124 and the antisense strand comprises SEQ ID NO: 164;
mm. the sense strand comprises SEQ ID NO: 124 and the antisense strand comprises SEQ ID NO: 165;
nn. the sense strand comprises SEQ ID NO: 124 and the antisense strand comprises SEQ ID NO: 166;
oo. the sense strand comprises SEQ ID NO: 124 and the antisense strand comprises SEQ ID NO: 134;
pp. the sense strand comprises SEQ ID NO: 125 and the antisense strand comprises SEQ ID NO: 162;
qq. the sense strand comprises SEQ ID NO: 125 and the antisense strand comprises SEQ ID NO: 163;
rr. the sense strand comprises SEQ ID NO: 125 and the antisense strand comprises SEQ ID NO: 164;
ss. the sense strand comprises SEQ ID NO: 125 and the antisense strand comprises SEQ ID NO: 165;
tt. the sense strand comprises SEQ ID NO: 125 and the antisense strand comprises SEQ ID NO: 166;
uu. the sense strand comprises SEQ ID NO: 125 and the antisense strand comprises SEQ ID NO: 134;
vv. the sense strand comprises SEQ ID NO: 126 and the antisense strand comprises SEQ ID NO: 162;
ww. the sense strand comprises SEQ ID NO: 126 and the antisense strand comprises SEQ ID NO: 163;
xx. the sense strand comprises SEQ ID NO: 126 and the antisense strand comprises SEQ ID NO: 164;
yy. the sense strand comprises SEQ ID NO: 126 and the antisense strand comprises SEQ ID NO: 165;
zz. the sense strand comprises SEQ ID NO: 126 and the antisense strand comprises SEQ ID NO: 166;
aaa. the sense strand comprises SEQ ID NO: 126 and the antisense strand comprises SEQ ID NO: 134;
bbb. the sense strand comprises SEQ ID NO: 127 and the antisense strand comprises SEQ ID NO: 167;
ccc. the sense strand comprises SEQ ID NO: 127 and the antisense strand comprises SEQ ID NO: 168;
ddd. the sense strand comprises SEQ ID NO: 127 and the antisense strand comprises SEQ ID NO: 169;
eee. the sense strand comprises SEQ ID NO: 128 and the antisense strand comprises SEQ ID NO: 170;
fff. the sense strand comprises SEQ ID NO: 128 and the antisense strand comprises SEQ ID NO: 171;
ggg. the sense strand comprises SEQ ID NO: 128 and the antisense strand comprises SEQ ID NO: 172;
hhh. the sense strand comprises SEQ ID NO: 129 and the antisense strand comprises SEQ ID NO: 173;
iii. the sense strand comprises SEQ ID NO: 129 and the antisense strand comprises SEQ ID NO: 174;
jjj. the sense strand comprises SEQ ID NO: 129 and the antisense strand comprises SEQ ID NO: 175;
kkk. the sense strand comprises SEQ ID NO: 130 and the antisense strand comprises SEQ ID NO: 176;
lll. the sense strand comprises SEQ ID NO: 130 and the antisense strand comprises SEQ ID NO: 177;
mmm. the sense strand comprises SEQ ID NO: 130 and the antisense strand comprises SEQ ID NO: 178;
nnn. the sense strand comprises SEQ ID NO: 131 and the antisense strand comprises SEQ ID NO: 179;
ooo. the sense strand comprises SEQ ID NO: 131 and the antisense strand comprises SEQ ID NO: 180;
ppp. the sense strand comprises SEQ ID NO: 131 and the antisense strand comprises SEQ ID NO: 181;
cccc. the sense strand comprises SEQ ID NO: 94 and the antisense strand comprises SEQ ID NO: 182;
dddd. the sense strand comprises SEQ ID NO: 95 and the antisense strand comprises SEQ ID NO: 183;
eeee. the sense strand comprises SEQ ID NO: 96 and the antisense strand comprises SEQ ID NO: 184;
ffff. the sense strand comprises SEQ ID NO: 97 and the antisense strand comprises SEQ ID NO: 185;
gggg. the sense strand comprises SEQ ID NO: 98 and the antisense strand comprises SEQ ID NO: 186;
hhhh. the sense strand comprises SEQ ID NO: 99 and the antisense strand comprises SEQ ID NO: 187;
iiii. the sense strand comprises SEQ ID NO: 100 and the antisense strand comprises SEQ ID NO: 188;
jjjj. the sense strand comprises SEQ ID NO: 101 and the antisense strand comprises SEQ ID NO: 189;
kkkk, the sense strand comprises SEQ ID NO: 102 and the antisense strand comprises SEQ ID NO: 190;
llll. the sense strand comprises SEQ ID NO: 103 and the antisense strand comprises SEQ ID NO: 191;
mmmm. the sense strand comprises SEQ ID NO: 104 and the antisense strand comprises SEQ ID NO: 192;
nnnn. the sense strand comprises SEQ ID NO: 105 and the antisense strand comprises SEQ ID NO: 193;
oooo. the sense strand comprises SEQ ID NO: 106 and the antisense strand comprises SEQ ID NO: 194;
pppp. the sense strand comprises SEQ ID NO: 107 and the antisense strand comprises SEQ ID NO: 195;
qqqq. the sense strand comprises SEQ ID NO: 108 and the antisense strand comprises SEQ ID NO: 196;
rrrr. the sense strand comprises SEQ ID NO: 109 and the antisense strand comprises SEQ ID NO: 197;
ssss. the sense strand comprises SEQ ID NO: 110 and the antisense strand comprises SEQ ID NO: 198;
tttt. the sense strand comprises SEQ ID NO: 111 and the antisense strand comprises SEQ ID NO: 199;
uuuu. the sense strand comprises SEQ ID NO: 112 and the antisense strand comprises SEQ ID NO: 200;
vvvv. the sense strand comprises SEQ ID NO: 113 and the antisense strand comprises SEQ ID NO: 201;
wwww. the sense strand comprises SEQ ID NO: 114 and the antisense strand comprises SEQ ID NO: 202;
xxxx. the sense strand comprises SEQ ID NO: 115 and the antisense strand comprises SEQ ID NO: 203;
yyyy. the sense strand comprises SEQ ID NO: 116 and the antisense strand comprises SEQ ID NO: 204;
zzzz. the sense strand comprises SEQ ID NO: 117 and the antisense strand comprises SEQ ID NO: 205;
aaaaa. the sense strand comprises SEQ ID NO: 118 and the antisense strand comprises SEQ ID NO: 206;
bbbbb. the sense strand comprises SEQ ID NO: 119 and the antisense strand comprises SEQ ID NO: 207;
ccccc. the sense strand comprises SEQ ID NO: 120 and the antisense strand comprises SEQ ID NO: 208;
ddddd. the sense strand comprises SEQ ID NO: 121 and the antisense strand comprises SEQ ID NO: 209;
eeeee. the sense strand comprises SEQ ID NO: 122 and the antisense strand comprises SEQ ID NO: 210;
fffff. the sense strand comprises SEQ ID NO: 123 and the antisense strand comprises SEQ ID NO: 211;
ggggg the sense strand comprises SEQ ID NO: 96 and the antisense strand comprises SEQ ID NO: 212;
hhhhh. the sense strand comprises SEQ ID NO: 96 and the antisense strand comprises SEQ ID NO: 213;
iiiii. the sense strand comprises SEQ ID NO: 96 and the antisense strand comprises SEQ ID NO: 214;
jjjjj. the sense strand comprises SEQ ID NO: 96 and the antisense strand comprises SEQ ID NO: 215;
kkkkk. the sense strand comprises SEQ ID NO: 96 and the antisense strand comprises SEQ ID NO: 216;
lllll. the sense strand comprises SEQ ID NO: 124 and the antisense strand comprises SEQ ID NO:212;
mmmmm. the sense strand comprises SEQ ID NO: 124 and the antisense strand comprises SEQ ID NO: 213;
nnnnn. the sense strand comprises SEQ ID NO: 124 and the antisense strand comprises SEQ ID NO: 214;
ooooo. the sense strand comprises SEQ ID NO: 124 and the antisense strand comprises SEQ ID NO: 215;
ppppp. the sense strand comprises SEQ ID NO: 124 and the antisense strand comprises SEQ ID NO: 216;
qqqqq. the sense strand comprises SEQ ID NO: 124 and the antisense strand comprises SEQ ID NO: 184;
rrrrr, the sense strand comprises SEQ ID NO: 125 and the antisense strand comprises SEQ ID NO: 212;
sssss. the sense strand comprises SEQ ID NO: 125 and the antisense strand comprises SEQ ID NO: 213;
ttttt, the sense strand comprises SEQ ID NO: 125 and the antisense strand comprises SEQ ID NO: 214;
uuuuu, the sense strand comprises SEQ ID NO: 125 and the antisense strand comprises SEQ ID NO: 215;
vvvvv. the sense strand comprises SEQ ID NO: 125 and the antisense strand comprises SEQ ID NO: 216;
wwwww, the sense strand comprises SEQ ID NO: 125 and the antisense strand comprises SEQ ID NO: 184;
xxxxx, the sense strand comprises SEQ ID NO: 126 and the antisense strand comprises SEQ ID NO: 212;
yyyyy, the sense strand comprises SEQ ID NO: 126 and the antisense strand comprises SEQ ID NO: 213;
zzzzz. the sense strand comprises SEQ ID NO: 126 and the antisense strand comprises SEQ ID NO: 214;
aaaaaa, the sense strand comprises SEQ ID NO: 126 and the antisense strand comprises SEQ ID NO: 215;
bbbbbb, the sense strand comprises SEQ ID NO: 126 and the antisense strand comprises SEQ ID NO: 216;
cccccc, the sense strand comprises SEQ ID NO: 126 and the antisense strand comprises SEQ ID NO: 184;
dddddd, the sense strand comprises SEQ ID NO: 127 and the antisense strand comprises SEQ ID NO: 217;
eeeeee, the sense strand comprises SEQ ID NO: 127 and the antisense strand comprises SEQ ID NO: 218;
ffffff, the sense strand comprises SEQ ID NO: 127 and the antisense strand comprises SEQ ID NO: 219;
gggggg, the sense strand comprises SEQ ID NO: 128 and the antisense strand comprises SEQ ID NO: 220;
hhhhhh. the sense strand comprises SEQ ID NO: 128 and the antisense strand comprises SEQ ID NO: 221;
iiiiii. the sense strand comprises SEQ ID NO: 128 and the antisense strand comprises SEQ ID NO: 222;
jjjjjj. the sense strand comprises SEQ ID NO: 129 and the antisense strand comprises SEQ ID NO: 223;
kkkkkk. the sense strand comprises SEQ ID NO: 129 and the antisense strand comprises SEQ ID NO: 224;
llllll. the sense strand comprises SEQ ID NO: 129 and the antisense strand comprises SEQ ID NO: 225;
mmmmmm. the sense strand comprises SEQ ID NO: 130 and the antisense strand comprises SEQ ID NO: 226;
nnnnnn. the sense strand comprises SEQ ID NO: 130 and the antisense strand comprises SEQ ID NO: 227;
oooooo. the sense strand comprises SEQ ID NO: 130 and the antisense strand comprises SEQ ID NO: 228;
pppppp. the sense strand comprises SEQ ID NO: 131 and the antisense strand comprises SEQ ID NO:229;
qqqqqq. the sense strand comprises SEQ ID NO: 131 and the antisense strand comprises SEQ ID NO: 230; or
rrrrrr. the sense strand comprises SEQ ID NO: 131 and the antisense strand comprises SEQ ID NO: 231.

34. The RNAi agent of claim 33, wherein:
a. the sense strand consists of SEQ ID NO: 94 and the antisense strand consists of SEQ ID NO: 132;
b. the sense strand consists of SEQ ID NO: 95 and the antisense strand consists of SEQ ID NO: 133;
c. the sense strand consists of SEQ ID NO: 96 and the antisense strand consists of SEQ ID NO: 134;
d. the sense strand consists of SEQ ID NO: 97 and the antisense strand consists of SEQ ID NO: 135;
e. the sense strand consists of SEQ ID NO: 98 and the antisense strand consists of SEQ ID NO: 136;
f. the sense strand consists of SEQ ID NO: 99 and the antisense strand consists of SEQ ID NO: 137;
g. the sense strand consists of SEQ ID NO: 100 and the antisense strand consists of SEQ ID NO: 138;
h. the sense strand consists of SEQ ID NO: 101 and the antisense strand consists of SEQ ID NO: 139;
i. the sense strand consists of SEQ ID NO: 102 and the antisense strand consists of SEQ ID NO: 140;
j. the sense strand consists of SEQ ID NO: 103 and the antisense strand consists of SEQ ID NO: 141;
k. the sense strand consists of SEQ ID NO: 104 and the antisense strand consists of SEQ ID NO: 142;
l. the sense strand consists of SEQ ID NO: 105 and the antisense strand consists of SEQ ID NO: 143;
m. the sense strand consists of SEQ ID NO: 106 and the antisense strand consists of SEQ ID NO: 144;
n. the sense strand consists of SEQ ID NO: 107 and the antisense strand consists of SEQ ID NO: 145;
o. the sense strand consists of SEQ ID NO: 108 and the antisense strand consists of SEQ ID NO: 146;
p. the sense strand consists of SEQ ID NO: 109 and the antisense strand consists of SEQ ID NO: 147;
q. the sense strand consists of SEQ ID NO: 110 and the antisense strand consists of SEQ ID NO: 148;
r. the sense strand consists of SEQ ID NO: 111 and the antisense strand consists of SEQ ID NO: 149;
s. the sense strand consists of SEQ ID NO: 112 and the antisense strand consists of SEQ ID NO: 150;
t. the sense strand consists of SEQ ID NO: 113 and the antisense strand consists of SEQ ID NO: 151;
u. the sense strand consists of SEQ ID NO: 114 and the antisense strand consists of SEQ ID NO: 152;
v. the sense strand consists of SEQ ID NO: 115 and the antisense strand consists of SEQ ID NO: 153;
w. the sense strand consists of SEQ ID NO: 116 and the antisense strand consists of SEQ ID NO: 154;
x. the sense strand consists of SEQ ID NO: 117 and the antisense strand consists of SEQ ID NO: 155;
y. the sense strand consists of SEQ ID NO: 118 and the antisense strand consists of SEQ ID NO: 156;
z. the sense strand consists of SEQ ID NO: 119 and the antisense strand consists of SEQ ID NO: 157;
aa. the sense strand consists of SEQ ID NO: 120 and the antisense strand consists of SEQ ID NO: 158;
bb. the sense strand consists of SEQ ID NO: 121 and the antisense strand consists of SEQ ID NO: 159;
cc. the sense strand consists of SEQ ID NO: 122 and the antisense strand consists of SEQ ID NO: 160;
dd. the sense strand consists of SEQ ID NO: 123 and the antisense strand consists of SEQ ID NO: 161;
ee. the sense strand consists of SEQ ID NO: 96 and the antisense strand consists of SEQ ID NO: 162;
ff. the sense strand consists of SEQ ID NO: 96 and the antisense strand consists of SEQ ID NO: 163;
gg. the sense strand consists of SEQ ID NO: 96 and the antisense strand consists of SEQ ID NO: 164;
hh. the sense strand consists of SEQ ID NO: 96 and the antisense strand consists of SEQ ID NO: 165;
ii. the sense strand consists of SEQ ID NO: 96 and the antisense strand consists of SEQ ID NO: 166;
jj. the sense strand consists of SEQ ID NO: 124 and the antisense strand consists of SEQ ID NO: 162;
kk. the sense strand consists of SEQ ID NO: 124 and the antisense strand consists of SEQ ID NO: 163;
ll. the sense strand consists of SEQ ID NO: 124 and the antisense strand consists of SEQ ID NO: 164;
mm. the sense strand consists of SEQ ID NO: 124 and the antisense strand consists of SEQ ID NO: 165;
nn. the sense strand consists of SEQ ID NO: 124 and the antisense strand consists of SEQ ID NO: 166;
ooo. the sense strand consists of SEQ ID NO: 124 and the antisense strand consists of SEQ ID NO: 134;
pp. the sense strand consists of SEQ ID NO: 125 and the antisense strand consists of SEQ ID NO: 162;
qq. the sense strand consists of SEQ ID NO: 125 and the antisense strand consists of SEQ ID NO: 163;
rr. the sense strand consists of SEQ ID NO: 125 and the antisense strand consists of SEQ ID NO: 164;
ss. the sense strand consists of SEQ ID NO: 125 and the antisense strand consists of SEQ ID NO: 165;
tt. the sense strand consists of SEQ ID NO: 125 and the antisense strand consists of SEQ ID NO: 166;
uu. the sense strand consists of SEQ ID NO: 125 and the antisense strand consists of SEQ ID NO: 134;
vv. the sense strand consists of SEQ ID NO: 126 and the antisense strand consists of SEQ ID NO: 162;
ww. the sense strand consists of SEQ ID NO: 126 and the antisense strand consists of SEQ ID NO:163;
xx. the sense strand consists of SEQ ID NO: 126 and the antisense strand consists of SEQ ID NO: 164;
yy. the sense strand consists of SEQ ID NO: 126 and the antisense strand consists of SEQ ID NO: 165;
zz. the sense strand consists of SEQ ID NO: 126 and the antisense strand consists of SEQ ID NO: 166;
aaa. the sense strand consists of SEQ ID NO: 126 and the antisense strand consists of SEQ ID NO: 134;
bbb. the sense strand consists of SEQ ID NO: 127 and the antisense strand consists of SEQ ID NO: 167;
ccc. the sense strand consists of SEQ ID NO: 127 and the antisense strand consists of SEQ ID NO: 168;
ddd. the sense strand consists of SEQ ID NO: 127 and the antisense strand consists of SEQ ID NO: 169;
eee. the sense strand consists of SEQ ID NO: 128 and the antisense strand consists of SEQ ID NO: 170;
fff. the sense strand consists of SEQ ID NO: 128 and the antisense strand consists of SEQ ID NO: 171;
ggg. the sense strand consists of SEQ ID NO: 128 and the antisense strand consists of SEQ ID NO: 172;
hhh. the sense strand consists of SEQ ID NO: 129 and the antisense strand consists of SEQ ID NO: 173;
iii. the sense strand consists of SEQ ID NO: 129 and the antisense strand consists of SEQ ID NO: 174;
jjj. the sense strand consists of SEQ ID NO: 129 and the antisense strand consists of SEQ ID NO: 175;
kkk. the sense strand consists of SEQ ID NO: 130 and the antisense strand consists of SEQ ID NO: 176;
lll. the sense strand consists of SEQ ID NO: 130 and the antisense strand consists of SEQ ID NO: 177;
mmm. the sense strand consists of SEQ ID NO: 130 and the antisense strand consists of SEQ ID NO: 178;
nnn. the sense strand consists of SEQ ID NO: 131 and the antisense strand consists of SEQ ID NO: 179;
ooo. the sense strand consists of SEQ ID NO: 131 and the antisense strand consists of SEQ ID NO: 180;
ppp. the sense strand consists of SEQ ID NO: 131 and the antisense strand consists of SEQ ID NO: 181;
cccc. the sense strand consists of SEQ ID NO: 94 and the antisense strand consists of SEQ ID NO: 182;
dddd. the sense strand consists of SEQ ID NO: 95 and the antisense strand consists of SEQ ID NO: 183;
eeee. the sense strand consists of SEQ ID NO: 96 and the antisense strand consists of SEQ ID NO: 184;
ffff. the sense strand consists of SEQ ID NO: 97 and the antisense strand consists of SEQ ID NO: 185;
gggg. the sense strand consists of SEQ ID NO: 98 and the antisense strand consists of SEQ ID NO: 186;
hhhh. the sense strand consists of SEQ ID NO: 99 and the antisense strand consists of SEQ ID NO: 187;
iiii. the sense strand consists of SEQ ID NO: 100 and the antisense strand consists of SEQ ID NO: 188;
jjjj. the sense strand consists of SEQ ID NO: 101 and the antisense strand consists of SEQ ID NO: 189;
kkkk. the sense strand consists of SEQ ID NO: 102 and the antisense strand consists of SEQ ID NO: 190;
llll. the sense strand consists of SEQ ID NO: 103 and the antisense strand consists of SEQ ID NO: 191;
mmmm. the sense strand consists of SEQ ID NO: 104 and the antisense strand consists of SEQ ID NO: 192;
nnnn. the sense strand consists of SEQ ID NO: 105 and the antisense strand consists of SEQ ID NO: 193;
oooo. the sense strand consists of SEQ ID NO: 106 and the antisense strand consists of SEQ ID NO: 194;
pppp. the sense strand consists of SEQ ID NO: 107 and the antisense strand consists of SEQ ID NO: 195;
qqqq. the sense strand consists of SEQ ID NO: 108 and the antisense strand consists of SEQ ID NO: 196;
rrrr. the sense strand consists of SEQ ID NO: 109 and the antisense strand consists of SEQ ID NO: 197;
ssss. the sense strand consists of SEQ ID NO: 110 and the antisense strand consists of SEQ ID NO: 198;
tttt. the sense strand consists of SEQ ID NO: 111 and the antisense strand consists of SEQ ID NO: 199;
uuuu. the sense strand consists of SEQ ID NO: 112 and the antisense strand consists of SEQ ID NO: 200;
vvvv. the sense strand consists of o SEQ ID NO: 113 and the antisense strand consists of SEQ ID NO: 201;
wwww. the sense strand consists of SEQ ID NO: 114 and the antisense strand consists of SEQ ID NO: 202;
xxxx. the sense strand consists of SEQ ID NO: 115 and the antisense strand consists of SEQ ID NO: 203;
yyyy. the sense strand consists of SEQ ID NO: 116 and the antisense strand consists of SEQ ID NO: 204;
zzzz. the sense strand consists of SEQ ID NO: 117 and the antisense strand consists of SEQ ID NO: 205;
aaaaa. the sense strand consists of SEQ ID NO: 118 and the antisense strand consists of SEQ ID NO: 206;
bbbbb. the sense strand consists of SEQ ID NO: 119 and the antisense strand consists of SEQ ID NO: 207;
ccccc. the sense strand consists of SEQ ID NO: 120 and the antisense strand consists of SEQ ID NO: 208;
ddddd. the sense strand consists of SEQ ID NO: 121 and the antisense strand consists of SEQ ID NO: 209;
eeeee. the sense strand consists of SEQ ID NO: 122 and the antisense strand consists of SEQ ID NO: 210;
fffff. the sense strand consists of SEQ ID NO: 123 and the antisense strand consists of SEQ ID NO: 211;
gggg the sense strand consists of SEQ ID NO: 96 and the antisense strand consists of SEQ ID NO: 212;
hhhhh. the sense strand consists of SEQ ID NO: 96 and the antisense strand consists of SEQ ID NO: 213;
iiiii. the sense strand consists of SEQ ID NO: 96 and the antisense strand consists of SEQ ID NO: 214;
jjjjj. the sense strand consists of SEQ ID NO: 96 and the antisense strand consists of SEQ ID NO: 215;
kkkkk. the sense strand consists of SEQ ID NO: 96 and the antisense strand consists of SEQ ID NO: 216;
lllll. the sense strand consists of SEQ ID NO: 124 and the antisense strand consists of SEQ ID NO:212;
mmmmm. the sense strand consists of SEQ ID NO: 124 and the antisense strand consists of SEQ ID NO: 213;
nnnnn. the sense strand consists of SEQ ID NO: 124 and the antisense strand consists of SEQ ID NO: 214;
ooooo. the sense strand consists of SEQ ID NO: 124 and the antisense strand consists of SEQ ID NO: 215;
ppppp. the sense strand consists of SEQ ID NO: 124 and the antisense strand consists of SEQ ID NO: 216;
qqqqq. the sense strand consists of SEQ ID NO: 124 and the antisense strand consists of SEQ ID NO: 184;
rrrrr. the sense strand consists of SEQ ID NO: 125 and the antisense strand consists of SEQ ID NO:212;
sssss. the sense strand consists of SEQ ID NO: 125 and the antisense strand consists of SEQ ID NO: 213;
ttttt. the sense strand consists of SEQ ID NO: 125 and the antisense strand consists of SEQ ID NO: 214;
uuuuu. the sense strand comprises SEQ ID NO: 125 and the antisense strand comprises SEQ ID NO: 215;
vvvvv. the sense strand consists of SEQ ID NO: 125 and the antisense strand consists of SEQ ID NO: 216;
wwwww. the sense strand consists of SEQ ID NO: 125 and the antisense strand consists of SEQ ID NO: 184;
xxxxx. the sense strand consists of SEQ ID NO: 126 and the antisense strand consists of SEQ ID NO: 212;
yyyyy. the sense strand consists of SEQ ID NO: 126 and the antisense strand consists of SEQ ID NO: 213;
zzzzz. the sense strand consists of SEQ ID NO: 126 and the antisense strand consists of SEQ ID NO: 214;
aaaaaa. the sense strand consists of SEQ ID NO: 126 and the antisense strand consists of SEQ ID NO: 215;
bbbbbb. the sense strand consists of SEQ ID NO: 126 and the antisense strand consists of SEQ ID NO: 216;
cccccc. the sense strand consists of SEQ ID NO: 126 and the antisense strand consists of SEQ ID NO: 184;
dddddd. the sense strand consists of SEQ ID NO:127 and the antisense strand consists of SEQ ID NO: 217;
eeeeee. the sense strand consists of SEQ ID NO: 127 and the antisense strand consists of SEQ ID NO: 218;
ffffff. the sense strand consists of SEQ ID NO: 127 and the antisense strand consists of SEQ ID NO: 219;
ggggg. the sense strand consists of SEQ ID NO: 128 and the antisense strand consists of SEQ ID NO: 220;
hhhhhh. the sense strand consists of SEQ ID NO: 128 and the antisense strand consists of SEQ ID NO: 221;
iiiiii. the sense strand consists of SEQ ID NO: 128 and the antisense strand consists of SEQ ID NO: 222;
jjjjjj. the sense strand consists of SEQ ID NO: 129 and the antisense strand consists of SEQ ID NO: 223;
kkkkkk. the sense strand consists of SEQ ID NO: 129 and the antisense strand consists of SEQ ID NO: 224;
llllll. the sense strand consists of SEQ ID NO: 129 and the antisense strand consists of SEQ ID NO: 225;
mmmmmm. the sense strand consists of SEQ ID NO: 130 and the antisense strand consists of SEQ ID NO: 226;
nnnnnn. the sense strand consists of SEQ ID NO: 130 and the antisense strand consists of SEQ ID NO: 227;
oooooo. the sense strand consists of SEQ ID NO: 130 and the antisense strand consists of SEQ ID NO: 228;
pppppp. the sense strand consists of SEQ ID NO: 131 and the antisense strand consists of SEQ ID NO: 229;
qqqqqq. the sense strand consists of SEQ ID NO: 131 and the antisense strand consists of SEQ ID NO: 230; or
rrrrrr. the sense strand consists of SEQ ID NO: 131 and the antisense strand consists of SEQ ID NO: 231
ddddddd.

35-46. (canceled)

47. A pharmaceutical composition comprising the RNAi agent of claim 8, and one or more pharmaceutically acceptable excipients.

48. A method of treating pain in a patient in need thereof, comprising administering the RNAi agent of claim 8, or a pharmaceutical composition thereof.

49. The method of claim 48, wherein the RNAi agent of claim 8 or the pharmaceutical composition thereof is administered intrathecally or epidurally.


SEQUENCE LISTING (242 sequences)

SEQ ID NO: 1 - DNA, 6626 bp, Homo sapiens
  source 1..6626: mol_type = genomic DNA
ttcttgcttcacacactagcctcttggctagagaacagacatcagatggagtttcttctggctatgcctgaatgttaagctgaacgtatgttccaggagctcgtggtctccagtagaggcaatctgggatagaagagaagatatttcttacgtagaagacaagcaagattgagcagcaaagagtataaatacttcctgaagaagaatgagaagatggaattccccattggatccctcgaaactaacaacttccgtcgctttactccggagtcactggtggagatagagaagcaaattgctgccaagcagggaacaaagaaagccagagagaagcatagggagcagaaggaccaagaagagaagcctcggccccagctggacttgaaagcctgcaaccagctgcccaagttctatggtgagctcccagcagaactgatcggggagcccctggaggatctagatccgttctacagcacacaccggacatttatggtgctgaacaaagggaggaccatttcccggtttagtgccactcgggccctgtggctattcagtcctttcaacctgatcagaagaacggccatcaaagtgtctgtccactcgtggttcagtttatttattacggtcactattttggttaattgtgtgtgcatgacccgaactgaccttccagagaaaattgaatatgtcttcactgtcatttacacctttgaagccttgataaagatactggcaagaggattttgtctaaatgagttcacgtacctgagagatccttggaactggctggattttagcgtcattaccctggcatatgttggcacagcaatagatctccgtgggatctcaggcctgcggacattcagagttcttagagcattaaaaacagtttctgtgatcccaggcctgaaggtcattgtgggggccctgattcactcagtgaagaaactggctgatgtgaccatcctcaccatcttctgcctaagtgtttttgccttggtggggctgcaactcttcaagggcaacctcaaaaataaatgtgtcaagaatgacatggctgtcaatgagacaaccaactactcatctcacagaaaaccagatatctacataaataagcgaggcacttctgaccccttactgtgtggcaatggatctgactcaggccactgccctgatggttatatctgccttaaaacttctgacaacccggattttaactacaccagctttgattcctttgcttgggctttcctctcactgttccgcctcatgacacaggattcctgggaacgcctctaccagcagaccctgaggacttctgggaaaatctatatgatcttttttgtgctcgtaatcttcctgggatctttctacctggtcaacttgatcttggctgtagtcaccatggcgtatgaggagcagaaccaggcaaccactgatgaaattgaagcaaaggagaagaagttccaggaggccctcgagatgctccggaaggagcaggaggtgctagcagcactagggattgacacaacctctctccactcccacaatggatcacctttaacctccaaaaatgccagtgagagaaggcatagaataaagccaagagtgtcagagggctccacagaagacaacaaatcaccccgctctgatccttacaaccagcgcaggatgtcttttctaggcctcgcctctggaaaacgccgggctagtcatggcagtgtgttccatttccggtcccctggccgagatatctcactccctgagggagtcacagatgatggagtctttcctggagaccacgaaagccatcggggctctctgctgctgggtgggggtgctggccagcaaggccccctccctagaagccctcttcctcaacccagcaaccctgactccaggcatggagaagatgaacaccaaccgccgcccactagtgagcttgcccctggagctgtcgatgtctcggcattcgatgcaggacaaaagaagactttcttgtcagcagaatacttagatgaacctttccgggcccaaagggcaatgagtgttgtcagtatcataacctccgtccttgaggaactcgaggagtctgaacagaagtgcccaccctgcttgaccagcttgtctcagaagtatctgatctgggattgctgccccatgtgggtgaagctcaagacaattctctttgggcttgtgacggatccctttgcagagctcaccatcaccttgtgcatcgtggtgaacaccatcttcatggccatggagcaccatggcatgagccctaccttcgaagccatgctccagataggcaacatcgtctttaccatattttttactgctgaaatggtcttcaaaatcattgccttcgacccatactattatttccagaagaagtggaatatctttgactgcatcatcgtcactgtgagtctgctagagctgggcgtggccaagaagggaagcctgtctgtgctgcggagcttccgcttgctgcgcgtattcaagctggccaaatcctggcccaccttaaacacactcatcaagatcatcggaaactcagtgggggcactggggaacctcaccatcatcctggccatcattgtctttgtctttgctctggttggcaagcagctcctaggggaaaactaccgtaacaaccgaaaaaatatctccgcgccccatgaagactggccccgctggcacatgcacgacttcttccactctttcctcattgtcttccgtatcctctgtggagagtggattgagaacatgtgggcctgcatggaagttggccaaaaatccatatgcctcatccttttcttgacggtgatggtgctagggaacctggtggtgcttaacctgttcatcgccctgctattgaactctttcagtgctgacaacctcacagccccggaggacgatggggaggtgaacaacctgcaggtggccctggcacggatccaggtctttggccatcgtaccaaacaggctctttgcagcttcttcagcaggtcctgcccattcccccagcccaaggcagagcctgagctggtggtgaaactcccactctccagctccaaggctgagaaccacattgctgccaacactgccagggggagctctggagggctccaagctcccagaggccccagggatgagcacagtgacttcatcgctaatccgactgtgtgggtctctgtgcccattgctgagggtgaatctgatcttgatgacttggaggatgatggtggggaagatgctcagagcttccagcaggaagtgatccccaaaggacagcaggagcagctgcagcaagtcgagaggtgtggggaccacctgacacccaggagcccaggcactggaacatcttctgaggacctggctccatccctgggtgagacgtggaaagatgagtctgttcctcaggtccctgctgagggagtggacgacacaagctcctctgagggcagcacggtggactgcctagatcctgaggaaatcctgaggaagatccctgagctggcagatgacctggaagaaccagatgactgcttcacagaaggatgcattcgccactgtccctgctgcaaactggataccaccaagagtccatgggatgtgggctggcaggtgcgcaagacttgctaccgtatcgtggagcacagctggtttgagagcttcatcatcttcatgatcctgctcagcagtggatctctggcctttgaagactattacctggaccagaagcccacggtgaaagctttgctggagtacactgacagggtcttcacctttatctttgtgttcgagatgctgcttaagtgggtggcctatggcttcaaaaagtacttcaccaatgcctggtgctggctggacttcctcattgtgaatatctcactgataagtctcacagcgaagattctggaatattctgaagtggctcccatcaaagcccttcgaacccttcgcgctctgcggccactgcgggctctttctcgatttgaaggcatgcgggtggtggtggatgccctggtgggcgccatcccatccatcatgaatgtcctcctcgtctgcctcatcttctggctcatcttcagcatcatgggtgtgaacctcttcgcagggaagttttggaggtgcatcaactataccgatggagagttttcccttgtacctttgtcgattgtgaataacaagtctgactgcaagattcaaaactccactggcagcttcttctgggtcaatgtgaaagtcaactttgataatgttgcaatgggttaccttgcacttctgcaggtggcaacctttaaaggctggatggacattatgtatgcagctgttgattcccgggaggtcaacatgcaacccaagtgggaggacaacgtgtacatgtatttgtactttgtcatcttcatcatttttggaggcttcttcacactgaatctctttgttggggtcataattgacaacttcaatcaacagaaaaaaaagttagggggccaggacatcttcatgacagaggagcagaagaaatactacaatgccatgaagaagttgggctccaagaagccccagaagcccatcccacggcccctgaacaagttccagggttttgtctttgacatcgtgaccagacaagcttttgacatcaccatcatggtcctcatctgcctcaacatgatcaccatgatggtggagactgatgaccaaagtgaagaaaagacgaaaattctgggcaaaatcaaccagttctttgtggccgtcttcacaggcgaatgtgtcatgaagatgttcgctttgaggcagtactacttcacaaatggctggaatgtgtttgacttcattgtggtggttctctccattgcgagcctgattttttctgcaattcttaagtcacttcaaagttacttctccccaacgctcttcagagtcatccgcctggcccgaattggccgcatcctcagactgatccgagcggccaaggggatccgcacactgctctttgccctcatgatgtccctgcctgccctcttcaacatcgggctgttgctattccttgtcatgttcatctactctatcttcggtatgtccagctttccccatgtgaggtgggaggctggcatcgacgacatgttcaacttccagaccttcgccaacagcatgctgtgcctcttccagattaccacgtcggccggctgggatggcctcctcagccccatcctcaacacagggcccccctactgtgaccccaatctgcccaacagcaatggcaccagaggggactgtgggagcccagccgtaggcatcatcttcttcaccacctacatcatcatctccttcctcatcatggtcaacatgtacattgcagtgattctggagaacttcaatgtggccacggaggagagcactgagcccctgagtgaggacgactttgacatgttctatgagacctgggagaagtttgacccagaggccactcagtttattaccttttctgctctctcggactttgcagacactctctctggtcccctgagaatcccaaaacccaatcgaaatatactgatccagatggacctgcctttggtccctggagataagatccactgcttggacatcctttttgctttcaccaagaatgtcctaggagaatccggggagttggattctctgaaggcaaatatggaggagaagtttatggcaactaatctttcaaaatcatcctatgaaccaatagcaaccactctccgatggaagcaagaagacatttcagccactgtcattcaaaaggcctatcggagctatgtgctgcaccgctccatggcactctctaacaccccatgtgtgcccagagctgaggaggaggctgcatcactcccagatgaaggttttgttgcattcacagcaaatgaaaattgtgtactcccagacaaatctgaaactgcttctgccacatcattcccaccgtcctatgagagtgtcactagaggccttagtgatagagtcaacatgaggacatctagctcaatacaaaatgaagatgaagccaccagtatggagctgattgcccctgggccctagtgagaacactccagcctggatatgttcagttatgatgcgtccttctgctctgtgttaactctttcccattgcagatcacacccagctacagcctcaccaatgcatgccactggtcacaatgtcagaactgggcagggaatgcaactggtaaccacctttgaaaaaaccatcatacacaaataggagtcagaagctaagagcacttccactgtgatttcccttctgaatttcaatatcagatcatgggttctcttactctccagaaaatatcccttgtccttttatttttgttgtaatcagattatatttactgagacaaacttctcaggaccagaacttccaaagatatagaacaagaacattactgaaggtccaaggtagtcctctgtggagcaccatacagagattggcaatattatttaggattttgcatgactgcatgtgagagctgtcggacaactgctctgacccagggtaacacctgtggcctatgtcatgaaaagcctttgagatatccattaaaattaatatttttaaatgta

SEQ ID NO: 2 - RNA, 18 nt, synthetic construct
  source 1..18: mol_type = other RNA
taactacaccagctttga

SEQ ID NO: 3 - RNA, 18 nt, synthetic construct
  source 1..18: mol_type = other RNA
ttgtcagcagaatactta

SEQ ID NO: 4 - RNA, 18 nt, synthetic construct
  source 1..18: mol_type = other RNA
tcagcagaatacttagat

SEQ ID NO: 5 - RNA, 18 nt, synthetic construct
  source 1..18: mol_type = other RNA
gtgacttcatcgctaatc

SEQ ID NO: 6 - RNA, 18 nt, synthetic construct
  source 1..18: mol_type = other RNA
gcacagtgacttcatcgc

SEQ ID NO: 7 - RNA, 18 nt, synthetic construct
  source 1..18: mol_type = other RNA
tgtctaaatgagttcacg

SEQ ID NO: 8 - RNA, 18 nt, synthetic construct
  source 1..18: mol_type = other RNA
tttgaagccttgataaag

SEQ ID NO: 9 - RNA, 18 nt, synthetic construct
  source 1..18: mol_type = other RNA
atcctatgaaccaatagc

SEQ ID NO: 10 - RNA, 18 nt, synthetic construct
  source 1..18: mol_type = other RNA
actctttcagtgctgaca

SEQ ID NO: 11 - RNA, 18 nt, synthetic construct
  source 1..18: mol_type = other RNA
gaatctctttgttggggt

SEQ ID NO: 12 - RNA, 18 nt, synthetic construct
  source 1..18: mol_type = other RNA
atgaggacatctagctca

SEQ ID NO: 13 - RNA, 18 nt, synthetic construct
  source 1..18: mol_type = other RNA
ttacggtcactattttgg

SEQ ID NO: 14 - RNA, 18 nt, synthetic construct
  source 1..18: mol_type = other RNA
caattctctttgggcttg

SEQ ID NO: 15 - RNA, 18 nt, synthetic construct
  source 1..18: mol_type = other RNA
catgagccctaccttcga

SEQ ID NO: 16 - RNA, 18 nt, synthetic construct
  source 1..18: mol_type = other RNA
caatgagtgttgtcagta

SEQ ID NO: 17 - RNA, 18 nt, synthetic construct
  source 1..18: mol_type = other RNA
ttggcaagcagctcctag

SEQ ID NO: 18 - RNA, 18 nt, synthetic construct
  source 1..18: mol_type = other RNA
tcaccatcttctgcctaa

SEQ ID NO: 19 - RNA, 18 nt, synthetic construct
  source 1..18: mol_type = other RNA
cagatgatggagtctttc

SEQ ID NO: 20 - RNA, 18 nt, synthetic construct
  source 1..18: mol_type = other RNA
ggcatatgttggcacagc

SEQ ID NO: 21 - RNA, 18 nt, synthetic construct
  source 1..18: mol_type = other RNA
agtgttgtcagtatcata

SEQ ID NO: 22 - RNA, 18 nt, synthetic construct
  source 1..18: mol_type = other RNA
ctatgaaccaatagcaac

SEQ ID NO: 23 - RNA, 18 nt, synthetic construct
  source 1..18: mol_type = other RNA
tgttgtcagtatcataac

SEQ ID NO: 24 - RNA, 18 nt, synthetic construct
  source 1..18: mol_type = other RNA
ataacaagtctgactgca

SEQ ID NO: 25 - RNA, 18 nt, synthetic construct
  source 1..18: mol_type = other RNA
tcattaccctggcatatg

SEQ ID NO: 26 - RNA, 18 nt, synthetic construct
  source 1..18: mol_type = other RNA
actgacagggtcttcacc

SEQ ID NO: 27 - RNA, 18 nt, synthetic construct
  source 1..18: mol_type = other RNA
aagccttgataaagatac

SEQ ID NO: 28 - RNA, 18 nt, synthetic construct
  source 1..18: mol_type = other RNA
tgagagtgtcactagagg

SEQ ID NO: 29 - RNA, 18 nt, synthetic construct
  source 1..18: mol_type = other RNA
atttcagccactgtcatt

SEQ ID NO: 30 - RNA, 18 nt, synthetic construct
  source 1..18: mol_type = other RNA
gattgtgaataacaagtc

SEQ ID NO: 31 - RNA, 18 nt, synthetic construct
  source 1..18: mol_type = other RNA
tacggtcactattttggt

SEQ ID NO: 32 - RNA, 18 nt, synthetic construct
  source 1..18: mol_type = other RNA
  modified_base 8: mod_base = OTHER; note = Abasic nucleotide
tcagcagnatacttagat

SEQ ID NO: 33 - RNA, 21 nt, synthetic construct
  source 1..21: mol_type = other RNA
tttaactacaccagctttgaa

SEQ ID NO: 34 - RNA, 21 nt, synthetic construct
  source 1..21: mol_type = other RNA
tcttgtcagcagaatacttaa

SEQ ID NO: 35 - RNA, 21 nt, synthetic construct
  source 1..21: mol_type = other RNA
tgtcagcagaatacttagata

SEQ ID NO: 36 - RNA, 21 nt, synthetic construct
  source 1..21: mol_type = other RNA
cagtgacttcatcgctaatca

SEQ ID NO: 37 - RNA, 21 nt, synthetic construct
  source 1..21: mol_type = other RNA
gagcacagtgacttcatcgca

SEQ ID NO: 38 - RNA, 21 nt, synthetic construct
  source 1..21: mol_type = other RNA
tttgtctaaatgagttcacga

SEQ ID NO: 39 - RNA, 21 nt, synthetic construct
  source 1..21: mol_type = other RNA
cctttgaagccttgataaaga

SEQ ID NO: 40 - RNA, 21 nt, synthetic construct
  source 1..21: mol_type = other RNA
tcatcctatgaaccaatagca

SEQ ID NO: 41 - RNA, 21 nt, synthetic construct
  source 1..21: mol_type = other RNA
gaactctttcagtgctgacaa

SEQ ID NO: 42 - RNA, 21 nt, synthetic construct
  source 1..21: mol_type = other RNA
ctgaatctctttgttggggta

SEQ ID NO: 43 - RNA, 21 nt, synthetic construct
  source 1..21: mol_type = other RNA
acatgaggacatctagctcaa

SEQ ID NO: 44 - RNA, 21 nt, synthetic construct
  source 1..21: mol_type = other RNA
tattacggtcactattttgga

SEQ ID NO: 45 - RNA, 21 nt, synthetic construct
  source 1..21: mol_type = other RNA
gacaattctctttgggcttga

SEQ ID NO: 46 - RNA, 21 nt, synthetic construct
  source 1..21: mol_type = other RNA
ggcatgagccctaccttcgaa

SEQ ID NO: 47 - RNA, 21 nt, synthetic construct
  source 1..21: mol_type = other RNA
ggcaatgagtgttgtcagtaa

SEQ ID NO: 48 - RNA, 21 nt, synthetic construct
  source 1..21: mol_type = other RNA
ggttggcaagcagctcctaga

SEQ ID NO: 49 - RNA, 21 nt, synthetic construct
  source 1..21: mol_type = other RNA
cctcaccatcttctgcctaaa

SEQ ID NO: 50 - RNA, 21 nt, synthetic construct
  source 1..21: mol_type = other RNA
cacagatgatggagtctttca

SEQ ID NO: 51 - RNA, 21 nt, synthetic construct
  source 1..21: mol_type = other RNA
ctggcatatgttggcacagca

SEQ ID NO: 52 - RNA, 21 nt, synthetic construct
  source 1..21: mol_type = other RNA
tgagtgttgtcagtatcataa

SEQ ID NO: 53 - RNA, 21 nt, synthetic construct
  source 1..21: mol_type = other RNA
tcctatgaaccaatagcaaca

SEQ ID NO: 54 - RNA, 21 nt, synthetic construct
  source 1..21: mol_type = other RNA
agtgttgtcagtatcataaca

SEQ ID NO: 55 - RNA, 21 nt, synthetic construct
  source 1..21: mol_type = other RNA
gaataacaagtctgactgcaa

SEQ ID NO: 56 - RNA, 21 nt, synthetic construct
  source 1..21: mol_type = other RNA
cgtcattaccctggcatatga

SEQ ID NO: 57 - RNA, 21 nt, synthetic construct
  source 1..21: mol_type = other RNA
acactgacagggtcttcacca

SEQ ID NO: 58 - RNA, 21 nt, synthetic construct
  source 1..21: mol_type = other RNA
tgaagccttgataaagataca

SEQ ID NO: 59 - RNA, 21 nt, synthetic construct
  source 1..21: mol_type = other RNA
tatgagagtgtcactagagga

SEQ ID NO: 60 - RNA, 21 nt, synthetic construct
  source 1..21: mol_type = other RNA
acatttcagccactgtcatta

SEQ ID NO: 61 - RNA, 21 nt, synthetic construct
  source 1..21: mol_type = other RNA
tcgattgtgaataacaagtca

SEQ ID NO: 62 - RNA, 21 nt, synthetic construct
  source 1..21: mol_type = other RNA
attacggtcactattttggta

SEQ ID NO: 63 - RNA, 21 nt, synthetic construct
  source 1..21: mol_type = other RNA
  modified_base 10: mod_base = OTHER; note = Abasic nucleotide
tgtcagcagnatacttagata

SEQ ID NO: 64 - RNA, 23 nt, synthetic construct
  source 1..23: mol_type = other RNA
ttcaaagctggtgtagttaaaat

SEQ ID NO: 65 - RNA, 23 nt, synthetic construct
  source 1..23: mol_type = other RNA
ttaagtattctgctgacaagaaa

SEQ ID NO: 66 - RNA, 23 nt, synthetic construct
  source 1..23: mol_type = other RNA
tatctaagtattctgctgacaag

SEQ ID NO: 67 - RNA, 23 nt, synthetic construct
  source 1..23: mol_type = other RNA
tgattagcgatgaagtcactgtg

SEQ ID NO: 68 - RNA, 23 nt, synthetic construct
  source 1..23: mol_type = other RNA
tgcgatgaagtcactgtgctcat

SEQ ID NO: 69 - RNA, 23 nt, synthetic construct
  source 1..23: mol_type = other RNA
tcgtgaactcatttagacaaaat

SEQ ID NO: 70 - RNA, 23 nt, synthetic construct
  source 1..23: mol_type = other RNA
tctttatcaaggcttcaaaggtg

SEQ ID NO: 71 - RNA, 23 nt, synthetic construct
  source 1..23: mol_type = other RNA
tgctattggttcataggatgatt

SEQ ID NO: 72 - RNA, 23 nt, synthetic construct
  source 1..23: mol_type = other RNA
ttgtcagcactgaaagagttcaa

SEQ ID NO: 73 - RNA, 23 nt, synthetic construct
  source 1..23: mol_type = other RNA
taccccaacaaagagattcagtg

SEQ ID NO: 74 - RNA, 23 nt, synthetic construct
  source 1..23: mol_type = other RNA
ttgagctagatgtcctcatgttg

SEQ ID NO: 75 - RNA, 23 nt, synthetic construct
  source 1..23: mol_type = other RNA
tccaaaatagtgaccgtaataaa

SEQ ID NO: 76 - RNA, 23 nt, synthetic construct
  source 1..23: mol_type = other RNA
tcaagcccaaagagaattgtctt

SEQ ID NO: 77 - RNA, 23 nt, synthetic construct
  source 1..23: mol_type = other RNA
ttcgaaggtagggctcatgccat

SEQ ID NO: 78 - RNA, 23 nt, synthetic construct
  source 1..23: mol_type = other RNA
ttactgacaacactcattgccct

SEQ ID NO: 79 - RNA, 23 nt, synthetic construct
  source 1..23: mol_type = other RNA
tctaggagctgcttgccaaccag

SEQ ID NO: 80 - RNA, 23 nt, synthetic construct
  source 1..23: mol_type = other RNA
tttaggcagaagatggtgaggat

SEQ ID NO: 81 - RNA, 23 nt, synthetic construct
  source 1..23: mol_type = other RNA
tgaaagactccatcatctgtgac

SEQ ID NO: 82 - RNA, 23 nt, synthetic construct
  source 1..23: mol_type = other RNA
tgctgtgccaacatatgccaggg

SEQ ID NO: 83 - RNA, 23 nt, synthetic construct
  source 1..23: mol_type = other RNA
ttatgatactgacaacactcatt

SEQ ID NO: 84 - RNA, 23 nt, synthetic construct
  source 1..23: mol_type = other RNA
tgttgctattggttcataggatg

SEQ ID NO: 85 - RNA, 23 nt, synthetic construct
  source 1..23: mol_type = other RNA
tgttatgatactgacaacactca

SEQ ID NO: 86 - RNA, 23 nt, synthetic construct
  source 1..23: mol_type = other RNA
ttgcagtcagacttgttattcac

SEQ ID NO: 87 - RNA, 23 nt, synthetic construct
  source 1..23: mol_type = other RNA
tcatatgccagggtaatgacgct

SEQ ID NO: 88 - RNA, 23 nt, synthetic construct
  source 1..23: mol_type = other RNA
tggtgaagaccctgtcagtgtac

SEQ ID NO: 89 - RNA, 23 nt, synthetic construct
  source 1..23: mol_type = other RNA
tgtatctttatcaaggcttcaaa

SEQ ID NO: 90 - RNA, 23 nt, synthetic construct
  source 1..23: mol_type = other RNA
tcctctagtgacactctcatagg

SEQ ID NO: 91 - RNA, 23 nt, synthetic construct
  source 1..23: mol_type = other RNA
taatgacagtggctgaaatgtct

SEQ ID NO: 92 - RNA, 23 nt, synthetic construct
  source 1..23: mol_type = other RNA
tgacttgttattcacaatcgaca

SEQ ID NO: 93 - RNA, 23 nt, synthetic construct
  source 1..23: mol_type = other RNA
taccaaaatagtgaccgtaataa

SEQ ID NO: 94 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methyluridine with a 3'-phosphorothioate linkage
  modified_base 3: mod_base = um
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluorocytidine
  modified_base 12: mod_base = cm
  modified_base 13: mod_base = OTHER; note = 2'-O-methyladenosine
  modified_base 14: mod_base = gm
  modified_base 15: mod_base = cm
  modified_base 16: mod_base = um
  modified_base 17: mod_base = um
  modified_base 18: mod_base = um
  modified_base 19: mod_base = OTHER; note = 2'-O-methylguanosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
tttaactacaccagctttgaa

SEQ ID NO: 95 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylcytidine with a 3'-phosphorothioate linkage
  modified_base 3: mod_base = um
  modified_base 4: mod_base = um
  modified_base 5: mod_base = gm
  modified_base 6: mod_base = um
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluoroadenosine
  modified_base 12: mod_base = gm
  modified_base 13: mod_base = OTHER; note = 2'-O-methyladenosine
  modified_base 14: mod_base = OTHER; note = 2'-O-methyladenosine
  modified_base 15: mod_base = um
  modified_base 16: mod_base = OTHER; note = 2'-O-methyladenosine
  modified_base 17: mod_base = cm
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
tcttgtcagcagaatacttaa

SEQ ID NO: 96 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = gm
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = OTHER; note = 2'-O-methyladenosine
  modified_base 14: mod_base = cm
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
tgtcagcagaatacttagata

SEQ ID NO: 97 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = um
  modified_base 5: mod_base = gm
  modified_base 6: mod_base = OTHER; note = 2'-O-methyladenosine
  modified_base 7: mod_base = OTHER; note = 2'-fluorocytidine
  modified_base 8: mod_base = um
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluoroadenosine
  modified_base 12: mod_base = um
  modified_base 13: mod_base = cm
  modified_base 14: mod_base = gm
  modified_base 15: mod_base = cm
  modified_base 16: mod_base = um
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyluridine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
cagtgacttcatcgctaatca

SEQ ID NO: 98 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = cm
  modified_base 7: mod_base = OTHER; note = 2'-fluoroadenosine
  modified_base 8: mod_base = gm
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluoroadenosine
  modified_base 12: mod_base = cm
  modified_base 13: mod_base = um
  modified_base 14: mod_base = um
  modified_base 15: mod_base = cm
  modified_base 16: mod_base = OTHER; note = 2'-O-methyladenosine
  modified_base 17: mod_base = um
  modified_base 18: mod_base = cm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
gagcacagtgacttcatcgca

SEQ ID NO: 99 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methyluridine with a 3'-phosphorothioate linkage
  modified_base 3: mod_base = um
  modified_base 4: mod_base = gm
  modified_base 5: mod_base = um
  modified_base 6: mod_base = cm
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluorouridine
  modified_base 12: mod_base = gm
  modified_base 13: mod_base = OTHER; note = 2'-O-methyladenosine
  modified_base 14: mod_base = gm
  modified_base 15: mod_base = um
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
tttgtctaaatgagttcacga

SEQ ID NO: 100 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = um
  modified_base 4: mod_base = um
  modified_base 5: mod_base = um
  modified_base 6: mod_base = gm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = um
  modified_base 14: mod_base = gm
  modified_base 15: mod_base = OTHER; note = 2'-O-methyladenosine
  modified_base 16: mod_base = um
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
cctttgaagccttgataaaga

SEQ ID NO: 101 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = um
  modified_base 5: mod_base = cm
  modified_base 6: mod_base = cm
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluoroadenosine
  modified_base 12: mod_base = OTHER; note = 2'-O-methyladenosine
  modified_base 13: mod_base = cm
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = um
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
tcatcctatgaaccaatagca

SEQ ID NO: 102 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = OTHER; note = 2'-O-methyladenosine
  modified_base 4: mod_base = cm
  modified_base 5: mod_base = um
  modified_base 6: mod_base = cm
  modified_base 7: mod_base = OTHER; note = 2'-fluorouridine
  modified_base 8: mod_base = um
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluoroadenosine
  modified_base 12: mod_base = gm
  modified_base 13: mod_base = um
  modified_base 14: mod_base = gm
  modified_base 15: mod_base = cm
  modified_base 16: mod_base = um
  modified_base 17: mod_base = gm
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
gaactctttcagtgctgacaa

SEQ ID NO: 103 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = um
  modified_base 7: mod_base = OTHER; note = 2'-fluorocytidine
  modified_base 8: mod_base = um
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorouridine
  modified_base 12: mod_base = um
  modified_base 13: mod_base = gm
  modified_base 14: mod_base = um
  modified_base 15: mod_base = um
  modified_base 16: mod_base = gm
  modified_base 17: mod_base = gm
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
ctgaatctctttgttggggta

SEQ ID NO: 104 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = um
  modified_base 5: mod_base = gm
  modified_base 6: mod_base = OTHER; note = 2'-O-methyladenosine
  modified_base 7: mod_base = OTHER; note = 2'-fluoroguanosine
  modified_base 8: mod_base = gm
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluoroadenosine
  modified_base 12: mod_base = um
  modified_base 13: mod_base = cm
  modified_base 14: mod_base = um
  modified_base 15: mod_base = OTHER; note = 2'-O-methyladenosine
  modified_base 16: mod_base = gm
  modified_base 17: mod_base = cm
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
acatgaggacatctagctcaa

SEQ ID NO: 105 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methyladenosine with a 3'-phosphorothioate linkage
  modified_base 3: mod_base = um
  modified_base 4: mod_base = um
  modified_base 5: mod_base = OTHER; note = 2'-O-methyladenosine
  modified_base 6: mod_base = cm
  modified_base 7: mod_base = OTHER; note = 2'-fluoroguanosine
  modified_base 8: mod_base = gm
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluoroadenosine
  modified_base 12: mod_base = cm
  modified_base 13: mod_base = um
  modified_base 14: mod_base = OTHER; note = 2'-O-methyladenosine
  modified_base 15: mod_base = um
  modified_base 16: mod_base = um
  modified_base 17: mod_base = um
  modified_base 18: mod_base = um
  modified_base 19: mod_base = OTHER; note = 2'-O-methylguanosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
tattacggtcactattttgga

SEQ ID NO: 106 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = um
  modified_base 7: mod_base = OTHER; note = 2'-fluorouridine
  modified_base 8: mod_base = cm
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = um
  modified_base 14: mod_base = gm
  modified_base 15: mod_base = gm
  modified_base 16: mod_base = gm
  modified_base 17: mod_base = cm
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
gacaattctctttgggcttga

SEQ ID NO: 107 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = um
  modified_base 6: mod_base = gm
  modified_base 7: mod_base = OTHER; note = 2'-fluoroadenosine
  modified_base 8: mod_base = gm
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = cm
  modified_base 16: mod_base = um
  modified_base 17: mod_base = um
  modified_base 18: mod_base = cm
  modified_base 19: mod_base = OTHER; note = 2'-O-methylguanosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
ggcatgagccctaccttcgaa

SEQ ID NO: 108 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = um
  modified_base 7: mod_base = OTHER; note = 2'-fluoroguanosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = um
  modified_base 13: mod_base = um
  modified_base 14: mod_base = gm
  modified_base 15: mod_base = um
  modified_base 16: mod_base = cm
  modified_base 17: mod_base = OTHER; note = 2'-O-methyladenosine
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
ggcaatgagtgttgtcagtaa

SEQ ID NO: 109 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = um
  modified_base 5: mod_base = gm
  modified_base 6: mod_base = gm
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluorocytidine
  modified_base 12: mod_base = OTHER; note = 2'-O-methyladenosine
  modified_base 13: mod_base = gm
  modified_base 14: mod_base = cm
  modified_base 15: mod_base = um
  modified_base 16: mod_base = cm
  modified_base 17: mod_base = cm
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
ggttggcaagcagctcctaga

SEQ ID NO: 110 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorouridine
  modified_base 12: mod_base = um
  modified_base 13: mod_base = cm
  modified_base 14: mod_base = um
  modified_base 15: mod_base = gm
  modified_base 16: mod_base = cm
  modified_base 17: mod_base = cm
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
cctcaccatcttctgcctaaa

SEQ ID NO: 111 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = OTHER; note = 2'-O-methyladenosine
  modified_base 5: mod_base = gm
  modified_base 6: mod_base = OTHER; note = 2'-O-methyladenosine
  modified_base 7: mod_base = OTHER; note = 2'-fluorouridine
  modified_base 8: mod_base = gm
  modified_base 9: mod_base = OTHER; note = 2'-fluoroadenosine
  modified_base 10: mod_base = OTHER; note = 2'-fluorouridine
  modified_base 11: mod_base = OTHER; note = 2'-fluoroguanosine
  modified_base 12: mod_base = gm
  modified_base 13: mod_base = OTHER; note = 2'-O-methyladenosine
  modified_base 14: mod_base = gm
  modified_base 15: mod_base = um
  modified_base 16: mod_base = cm
  modified_base 17: mod_base = um
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
cacagatgatggagtctttca

SEQ ID NO: 112 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = gm
  modified_base 5: mod_base = cm
  modified_base 6: mod_base = OTHER; note = 2'-O-methyladenosine
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluorouridine
  modified_base 12: mod_base = um
  modified_base 13: mod_base = gm
  modified_base 14: mod_base = gm
  modified_base 15: mod_base = cm
  modified_base 16: mod_base = OTHER; note = 2'-O-methyladenosine
  modified_base 17: mod_base = cm
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
ctggcatatgttggcacagca

SEQ ID NO: 113 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = OTHER; note = 2'-O-methyladenosine
  modified_base 4: mod_base = gm
  modified_base 5: mod_base = um
  modified_base 6: mod_base = gm
  modified_base 7: mod_base = OTHER; note = 2'-fluorouridine
  modified_base 8: mod_base = um
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-O-methyladenosine
  modified_base 13: mod_base = gm
  modified_base 14: mod_base = um
  modified_base 15: mod_base = OTHER; note = 2'-O-methyladenosine
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
tgagtgttgtcagtatcataa

SEQ ID NO: 114 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylcytidine with a 3'-phosphorothioate linkage
  modified_base 3: mod_base = cm
  modified_base 4: mod_base = um
  modified_base 5: mod_base = OTHER; note = 2'-O-methyladenosine
  modified_base 6: mod_base = um
  modified_base 7: mod_base = OTHER; note = 2'-fluoroguanosine
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = OTHER; note = 2'-O-methyladenosine
  modified_base 14: mod_base = um
  modified_base 15: mod_base = OTHER; note = 2'-O-methyladenosine
  modified_base 16: mod_base = gm
  modified_base 17: mod_base = cm
  modified_base 18: mod_base = OTHER; note = 2'-O-methyladenosine
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
tcctatgaaccaatagcaaca

SEQ ID NO: 115 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = gm
  modified_base 5: mod_base = um
  modified_base 6: mod_base = um
  modified_base 7: mod_base = OTHER; note = 2'-fluoroguanosine
  modified_base 8: mod_base = um
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluoroguanosine
  modified_base 12: mod_base = um
  modified_base 13: mod_base = OTHER; note = 2'-O-methyladenosine
  modified_base 14: mod_base = um
  modified_base 15: mod_base = cm
  modified_base 16: mod_base = OTHER; note = 2'-O-methyladenosine
  modified_base 17: mod_base = um
  modified_base 18: mod_base = OTHER; note = 2'-O-methyladenosine
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
agtgttgtcagtatcataaca

SEQ ID NO: 116 - RNA, 21 nt, synthetic construct
  source 1..21: mol_type = other RNA
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = OTHER; note = 2'-O-methyladenosine
  modified_base 4: mod_base = um
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluorouridine
  modified_base 12: mod_base = cm
  modified_base 13: mod_base = um
  modified_base 14: mod_base = gm
  modified_base 15: mod_base = OTHER; note = 2'-O-methyladenosine
  modified_base 16: mod_base = cm
  modified_base 17: mod_base = um
  modified_base 18: mod_base = gm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
gaataacaagtctgactgcaa

SEQ ID NO: 117 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = um
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = gm
  modified_base 14: mod_base = gm
  modified_base 15: mod_base = cm
  modified_base 16: mod_base = OTHER; note = 2'-O-methyladenosine
  modified_base 17: mod_base = um
  modified_base 18: mod_base = OTHER; note = 2'-O-methyladenosine
  modified_base 19: mod_base = OTHER; note = 2'-O-methyluridine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
cgtcattaccctggcatatga

SEQ ID NO: 118 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = cm
  modified_base 5: mod_base = um
  modified_base 6: mod_base = gm
  modified_base 7: mod_base = OTHER; note = 2'-fluoroadenosine
  modified_base 8: mod_base = cm
  modified_base 9: mod_base = OTHER; note = 2'-fluoroadenosine
  modified_base 10: mod_base = OTHER; note = 2'-fluoroguanosine
  modified_base 11: mod_base = OTHER; note = 2'-fluoroguanosine
  modified_base 12: mod_base = gm
  modified_base 13: mod_base = um
  modified_base 14: mod_base = cm
  modified_base 15: mod_base = um
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
acactgacagggtcttcacca

SEQ ID NO: 119 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = cm
  modified_base 7: mod_base = OTHER; note = 2'-fluorocytidine
  modified_base 8: mod_base = um
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluoroadenosine
  modified_base 12: mod_base = um
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methyladenosine
  modified_base 16: mod_base = gm
  modified_base 17: mod_base = OTHER; note = 2'-O-methyladenosine
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
tgaagccttgataaagataca

SEQ ID NO: 120 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methyladenosine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = gm
  modified_base 7: mod_base = OTHER; note = 2'-fluoroadenosine
  modified_base 8: mod_base = gm
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluorouridine
  modified_base 12: mod_base = cm
  modified_base 13: mod_base = OTHER; note = 2'-O-methyladenosine
  modified_base 14: mod_base = cm
  modified_base 15: mod_base = um
  modified_base 16: mod_base = OTHER; note = 2'-O-methyladenosine
  modified_base 17: mod_base = gm
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
tatgagagtgtcactagagga

SEQ ID NO: 121 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = um
  modified_base 5: mod_base = um
  modified_base 6: mod_base = um
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluorocytidine
  modified_base 12: mod_base = OTHER; note = 2'-O-methyladenosine
  modified_base 13: mod_base = cm
  modified_base 14: mod_base = um
  modified_base 15: mod_base = gm
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
acatttcagccactgtcatta

SEQ ID NO: 122 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylcytidine with a 3'-phosphorothioate linkage
  modified_base 3: mod_base = gm
  modified_base 4: mod_base = OTHER; note = 2'-O-methyladenosine
  modified_base 5: mod_base = um
  modified_base 6: mod_base = um
  modified_base 7: mod_base = OTHER; note = 2'-fluoroguanosine
  modified_base 8: mod_base = um
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = OTHER; note = 2'-O-methyladenosine
  modified_base 14: mod_base = OTHER; note = 2'-O-methyladenosine
  modified_base 15: mod_base = cm
  modified_base 16: mod_base = OTHER; note = 2'-O-methyladenosine
  modified_base 17: mod_base = OTHER; note = 2'-O-methyladenosine
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
tcgattgtgaataacaagtca

SEQ ID NO: 123 - RNA, 21 nt, synthetic construct
  source 1..21: mol_type = other RNA
  modified_base 1: mod_base = OTHER; note = 2'-O-methyladenosine with a 3'-phosphorothioate linkage
  modified_base 2: mod_base = OTHER; note = 2'-O-methyluridine with a 3'-phosphorothioate linkage
  modified_base 3: mod_base = um
  modified_base 4: mod_base = OTHER; note = 2'-O-methyladenosine
  modified_base 5: mod_base = cm
  modified_base 6: mod_base = gm
  modified_base 7: mod_base = OTHER; note = 2'-fluoroguanosine
  modified_base 8: mod_base = um
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = um
  modified_base 15: mod_base = um
  modified_base 16: mod_base = um
  modified_base 17: mod_base = um
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
attacggtcactattttggta

SEQ ID NO: 124 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = OTHER; note = 2'-O-methyladenosine
  modified_base 14: mod_base = cm
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
tgtcagcagaatacttagata

SEQ ID NO: 125 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = gm
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluoroguanosine
  modified_base 10: mod_base = OTHER; note = Abasic nucleotide
  modified_base 11: mod_base = OTHER; note = 2'-fluoroadenosine
  modified_base 12: mod_base = um
  modified_base 13: mod_base = OTHER; note = 2'-O-methyladenosine
  modified_base 14: mod_base = cm
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
tgtcagcagnatacttagata

SEQ ID NO: 126 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluoroguanosine
  modified_base 10: mod_base = OTHER; note = Abasic nucleotide
  modified_base 11: mod_base = OTHER; note = 2'-fluoroadenosine
  modified_base 12: mod_base = um
  modified_base 13: mod_base = OTHER; note = 2'-O-methyladenosine
  modified_base 14: mod_base = cm
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
tgtcagcagnatacttagata

SEQ ID NO: 127 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methyluridine with a 3'-phosphorothioate linkage
  modified_base 3: mod_base = um
  modified_base 4: mod_base = gm
  modified_base 5: mod_base = um
  modified_base 6: mod_base = cm
  modified_base 7: mod_base = um
  modified_base 8: mod_base = OTHER; note = 2'-O-methyladenosine
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluorouridine
  modified_base 12: mod_base = gm
  modified_base 13: mod_base = OTHER; note = 2'-O-methyladenosine
  modified_base 14: mod_base = gm
  modified_base 15: mod_base = um
  modified_base 16: mod_base = um
  modified_base 17: mod_base = cm
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
tttgtctaaatgagttcacga

SEQ ID NO: 128 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methyluridine with a 3'-phosphorothioate linkage
  modified_base 3: mod_base = um
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = um
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluorocytidine
  modified_base 12: mod_base = cm
  modified_base 13: mod_base = OTHER; note = 2'-O-methyladenosine
  modified_base 14: mod_base = gm
  modified_base 15: mod_base = cm
  modified_base 16: mod_base = um
  modified_base 17: mod_base = um
  modified_base 18: mod_base = um
  modified_base 19: mod_base = OTHER; note = 2'-O-methylguanosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
tttaactacaccagctttgaa

SEQ ID NO: 129 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylcytidine with a 3'-phosphorothioate linkage
  modified_base 3: mod_base = um
  modified_base 4: mod_base = um
  modified_base 5: mod_base = gm
  modified_base 6: mod_base = um
  modified_base 7: mod_base = cm
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluoroadenosine
  modified_base 12: mod_base = gm
  modified_base 13: mod_base = OTHER; note = 2'-O-methyladenosine
  modified_base 14: mod_base = OTHER; note = 2'-O-methyladenosine
  modified_base 15: mod_base = um
  modified_base 16: mod_base = OTHER; note = 2'-O-methyladenosine
  modified_base 17: mod_base = cm
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
tcttgtcagcagaatacttaa

SEQ ID NO: 130 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = um
  modified_base 5: mod_base = cm
  modified_base 6: mod_base = cm
  modified_base 7: mod_base = um
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluoroadenosine
  modified_base 12: mod_base = OTHER; note = 2'-O-methyladenosine
  modified_base 13: mod_base = cm
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = um
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
tcatcctatgaaccaatagca

SEQ ID NO: 131 - RNA, 21 nt, synthetic construct
  source 1..21: mol_type = other RNA
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = um
  modified_base 4: mod_base = um
  modified_base 5: mod_base = um
  modified_base 6: mod_base = gm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = um
  modified_base 14: mod_base = gm
  modified_base 15: mod_base = OTHER; note = 2'-O-methyladenosine
  modified_base 16: mod_base = um
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
cctttgaagccttgataaaga

SEQ ID NO: 132 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = cm
  modified_base 9: mod_base = um
  modified_base 10: mod_base = gm
  modified_base 11: mod_base = gm
  modified_base 12: mod_base = um
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
ttcaaagctggtgtagttaaaat

SEQ ID NO: 133 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = OTHER; note = 2'-fluorouridine
  modified_base 7: mod_base = OTHER; note = 2'-O-methyladenosine
  modified_base 8: mod_base = um
  modified_base 9: mod_base = um
  modified_base 10: mod_base = cm
  modified_base 11: mod_base = um
  modified_base 12: mod_base = gm
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
ttaagtattctgctgacaagaaa

SEQ ID NO: 134 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = um
  modified_base 4: mod_base = cm
  modified_base 5: mod_base = um
  modified_base 6: mod_base = OTHER; note = 2'-fluoroadenosine
  modified_base 7: mod_base = OTHER; note = 2'-O-methyladenosine
  modified_base 8: mod_base = gm
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 135 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = um
  modified_base 5: mod_base = um
  modified_base 6: mod_base = OTHER; note = 2'-fluoroadenosine
  modified_base 7: mod_base = gm
  modified_base 8: mod_base = cm
  modified_base 9: mod_base = gm
  modified_base 10: mod_base = OTHER; note = 2'-O-methyladenosine
  modified_base 11: mod_base = um
  modified_base 12: mod_base = gm
  modified_base 13: mod_base = OTHER; note = 2'-O-methyladenosine
  modified_base 14: mod_base = OTHER; note = 2'-fluoroadenosine
  modified_base 15: mod_base = gm
  modified_base 16: mod_base = OTHER; note = 2'-fluorouridine
  modified_base 17: mod_base = cm
  modified_base 18: mod_base = OTHER; note = 2'-O-methyladenosine
  modified_base 19: mod_base = cm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = gm
tgattagcgatgaagtcactgtg

SEQ ID NO: 136 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = cm
  modified_base 4: mod_base = gm
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = OTHER; note = 2'-O-methyladenosine
  modified_base 9: mod_base = OTHER; note = 2'-O-methyladenosine
  modified_base 10: mod_base = gm
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluorocytidine
  modified_base 15: mod_base = um
  modified_base 16: mod_base = OTHER; note = 2'-fluoroguanosine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = um
tgcgatgaagtcactgtgctcat

SEQ ID NO: 137 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = gm
  modified_base 4: mod_base = um
  modified_base 5: mod_base = gm
  modified_base 6: mod_base = OTHER; note = 2'-fluoroadenosine
  modified_base 7: mod_base = OTHER; note = 2'-O-methyladenosine
  modified_base 8: mod_base = cm
  modified_base 9: mod_base = um
  modified_base 10: mod_base = cm
  modified_base 11: mod_base = OTHER; note = 2'-O-methyladenosine
  modified_base 12: mod_base = um
  modified_base 13: mod_base = um
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
tcgtgaactcatttagacaaaat

SEQ ID NO: 138 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = um
  modified_base 4: mod_base = um
  modified_base 5: mod_base = um
  modified_base 6: mod_base = OTHER; note = 2'-fluoroadenosine
  modified_base 7: mod_base = um
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = gm
  modified_base 13: mod_base = cm
  modified_base 14: mod_base = OTHER; note = 2'-fluorouridine
  modified_base 15: mod_base = um
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = gm
tctttatcaaggcttcaaaggtg

SEQ ID NO: 139 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = cm
  modified_base 4: mod_base = um
  modified_base 5: mod_base = OTHER; note = 2'-O-methyladenosine
  modified_base 6: mod_base = OTHER; note = 2'-fluorouridine
  modified_base 7: mod_base = um
  modified_base 8: mod_base = gm
  modified_base 9: mod_base = gm
  modified_base 10: mod_base = um
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = um
tgctattggttcataggatgatt

SEQ ID NO: 140 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = gm
  modified_base 4: mod_base = um
  modified_base 5: mod_base = cm
  modified_base 6: mod_base = OTHER; note = 2'-fluoroadenosine
  modified_base 7: mod_base = gm
  modified_base 8: mod_base = cm
  modified_base 9: mod_base = OTHER; note = 2'-O-methyladenosine
  modified_base 10: mod_base = cm
  modified_base 11: mod_base = um
  modified_base 12: mod_base = gm
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = gm
  modified_base 19: mod_base = um
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
ttgtcagcactgaaagagttcaa

SEQ ID NO: 141 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = cm
  modified_base 4: mod_base = cm
  modified_base 5: mod_base = cm
  modified_base 6: mod_base = OTHER; note = 2'-fluorocytidine
  modified_base 7: mod_base = OTHER; note = 2'-O-methyladenosine
  modified_base 8: mod_base = OTHER; note = 2'-O-methyladenosine
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-O-methyladenosine
  modified_base 13: mod_base = gm
  modified_base 14: mod_base = OTHER; note = 2'-fluoroadenosine
  modified_base 15: mod_base = gm
  modified_base 16: mod_base = OTHER; note = 2'-fluoroadenosine
  modified_base 17: mod_base = um
  modified_base 18: mod_base = um
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = gm
taccccaacaaagagattcagtg

SEQ ID NO: 142 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = gm
  modified_base 4: mod_base = OTHER; note = 2'-O-methyladenosine
  modified_base 5: mod_base = gm
  modified_base 6: mod_base = OTHER; note = 2'-fluorocytidine
  modified_base 7: mod_base = um
  modified_base 8: mod_base = OTHER; note = 2'-O-methyladenosine
  modified_base 9: mod_base = gm
  modified_base 10: mod_base = OTHER; note = 2'-O-methyladenosine
  modified_base 11: mod_base = um
  modified_base 12: mod_base = gm
  modified_base 13: mod_base = um
  modified_base 14: mod_base = OTHER; note = 2'-fluorocytidine
  modified_base 15: mod_base = cm
  modified_base 16: mod_base = OTHER; note = 2'-fluorouridine
  modified_base 17: mod_base = cm
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methyluridine with a 3'-phosphorothioate linkage
  modified_base 22: mod_base = OTHER; note = 2'-O-methyluridine with a 3'-phosphorothioate linkage
  modified_base 23: mod_base = gm
ttgagctagatgtcctcatgttg

SEQ ID NO: 143 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = um
  modified_base 9: mod_base = OTHER; note = 2'-O-methyladenosine
  modified_base 10: mod_base = gm
  modified_base 11: mod_base = um
  modified_base 12: mod_base = gm
  modified_base 13: mod_base = OTHER; note = 2'-O-methyladenosine
  modified_base 14: mod_base = OTHER; note = 2'-fluorocytidine
  modified_base 15: mod_base = cm
  modified_base 16: mod_base = OTHER; note = 2'-fluoroguanosine
  modified_base 17: mod_base = um
  modified_base 18: mod_base = OTHER; note = 2'-O-methyladenosine
  modified_base 19: mod_base = OTHER; note = 2'-O-methyladenosine
  modified_base 20: mod_base = um
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tccaaaatagtgaccgtaataaa

SEQ ID NO: 144 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = OTHER; note = 2'-fluorocytidine
  modified_base 7: mod_base = cm
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = gm
  modified_base 13: mod_base = OTHER; note = 2'-O-methyladenosine
  modified_base 14: mod_base = OTHER; note = 2'-fluoroguanosine
  modified_base 15: mod_base = OTHER; note = 2'-O-methyladenosine
  modified_base 16: mod_base = OTHER; note = 2'-fluoroadenosine
  modified_base 17: mod_base = um
  modified_base 18: mod_base = um
  modified_base 19: mod_base = gm
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
tcaagcccaaagagaattgtctt

SEQ ID NO: 145 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = cm
  modified_base 4: mod_base = gm
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = gm
  modified_base 9: mod_base = um
  modified_base 10: mod_base = OTHER; note = 2'-O-methyladenosine
  modified_base 11: mod_base = gm
  modified_base 12: mod_base = gm
  modified_base 13: mod_base = gm
  modified_base 14: mod_base = OTHER; note = 2'-fluorocytidine
  modified_base 15: mod_base = um
  modified_base 16: mod_base = OTHER; note = 2'-fluorocytidine
  modified_base 17: mod_base = OTHER; note = 2'-O-methyladenosine
  modified_base 18: mod_base = um
  modified_base 19: mod_base = gm
  modified_base 20: mod_base = cm
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = um
ttcgaaggtagggctcatgccat

SEQ ID NO: 146 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = cm
  modified_base 5: mod_base = um
  modified_base 6: mod_base = OTHER; note = 2'-fluoroguanosine
  modified_base 7: mod_base = OTHER; note = 2'-O-methyladenosine
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methylcytidine with a 3'-phosphorothioate linkage
  modified_base 22: mod_base = OTHER; note = 2'-O-methylcytidine with a 3'-phosphorothioate linkage
  modified_base 23: mod_base = um
ttactgacaacactcattgccct

SEQ ID NO: 147 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = um
  modified_base 4: mod_base = OTHER; note = 2'-O-methyladenosine
  modified_base 5: mod_base = gm
  modified_base 6: mod_base = OTHER; note = 2'-fluoroguanosine
  modified_base 7: mod_base = OTHER; note = 2'-O-methyladenosine
  modified_base 8: mod_base = gm
  modified_base 9: mod_base = cm
  modified_base 10: mod_base = um
  modified_base 11: mod_base = gm
  modified_base 12: mod_base = cm
  modified_base 13: mod_base = um
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = cm
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = gm
tctaggagctgcttgccaaccag

SEQ ID NO: 148 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = um
  modified_base 4: mod_base = OTHER; note = 2'-O-methyladenosine
  modified_base 5: mod_base = gm
  modified_base 6: mod_base = OTHER; note = 2'-fluoroguanosine
  modified_base 7: mod_base = cm
  modified_base 8: mod_base = OTHER; note = 2'-O-methyladenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = gm
  modified_base 13: mod_base = OTHER; note = 2'-O-methyladenosine
  modified_base 14: mod_base = OTHER; note = 2'-fluorouridine
  modified_base 15: mod_base = gm
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = um
tttaggcagaagatggtgaggat

SEQ ID NO: 149 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyladenosine
  modified_base 8: mod_base = cm
  modified_base 9: mod_base = um
  modified_base 10: mod_base = cm
  modified_base 11: mod_base = cm
  modified_base 12: mod_base = OTHER; note = 2'-O-methyladenosine
  modified_base 13: mod_base = um
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluorouridine
  modified_base 17: mod_base = cm
  modified_base 18: mod_base = um
  modified_base 19: mod_base = gm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = cm
tgaaagactccatcatctgtgac

SEQ ID NO: 150 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = cm
  modified_base 4: mod_base = um
  modified_base 5: mod_base = gm
  modified_base 6: mod_base = OTHER; note = 2'-fluorouridine
  modified_base 7: mod_base = gm
  modified_base 8: mod_base = cm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = gm
  modified_base 18: mod_base = cm
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = gm
tgctgtgccaacatatgccaggg

SEQ ID NO: 151 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = um
  modified_base 5: mod_base = gm
  modified_base 6: mod_base = OTHER; note = 2'-fluoroadenosine
  modified_base 7: mod_base = um
  modified_base 8: mod_base = OTHER; note = 2'-O-methyladenosine
  modified_base 9: mod_base = cm
  modified_base 10: mod_base = um
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluorocytidine
  modified_base 17: mod_base = OTHER; note = 2'-O-methyladenosine
  modified_base 18: mod_base = cm
  modified_base 19: mod_base = um
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = um
ttatgatactgacaacactcatt

SEQ ID NO: 152 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = um
  modified_base 4: mod_base = um
  modified_base 5: mod_base = gm
  modified_base 6: mod_base = OTHER; note = 2'-fluorocytidine
  modified_base 7: mod_base = um
  modified_base 8: mod_base = OTHER; note = 2'-O-methyladenosine
  modified_base 9: mod_base = um
  modified_base 10: mod_base = um
  modified_base 11: mod_base = gm
  modified_base 12: mod_base = gm
  modified_base 13: mod_base = um
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = OTHER; note = 2'-O-methyladenosine
  modified_base 19: mod_base = gm
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = gm
tgttgctattggttcataggatg

SEQ ID NO: 153 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = um
  modified_base 4: mod_base = um
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = OTHER; note = 2'-O-methyladenosine
  modified_base 9: mod_base = um
  modified_base 10: mod_base = OTHER; note = 2'-O-methyladenosine
  modified_base 11: mod_base = cm
  modified_base 12: mod_base = um
  modified_base 13: mod_base = gm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = OTHER; note = 2'-O-methyladenosine
tgttatgatactgacaacactca

SEQ ID NO: 154 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-fluoroguanosine
  modified_base 7: mod_base = um
  modified_base 8: mod_base = cm
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = cm
  modified_base 13: mod_base = um
  modified_base 14: mod_base = OTHER; note = 2'-fluorouridine
  modified_base 15: mod_base = gm
  modified_base 16: mod_base = OTHER; note = 2'-fluorouridine
  modified_base 17: mod_base = um
  modified_base 18: mod_base = OTHER; note = 2'-O-methyladenosine
  modified_base 19: mod_base = um
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = cm
ttgcagtcagacttgttattcac

SEQ ID NO: 155 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = um
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = cm
  modified_base 9: mod_base = cm
  modified_base 10: mod_base = OTHER; note = 2'-O-methyladenosine
  modified_base 11: mod_base = gm
  modified_base 12: mod_base = gm
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroadenosine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methylguanosine with a 3'-phosphorothioate linkage
  modified_base 22: mod_base = OTHER; note = 2'-O-methylcytidine with a 3'-phosphorothioate linkage
  modified_base 23: mod_base = um
tcatatgccagggtaatgacgct

SEQ ID NO: 156 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = gm
  modified_base 4: mod_base = um
  modified_base 5: mod_base = gm
  modified_base 6: mod_base = OTHER; note = 2'-fluoroadenosine
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = cm
  modified_base 11: mod_base = cm
  modified_base 12: mod_base = cm
  modified_base 13: mod_base = um
  modified_base 14: mod_base = OTHER; note = 2'-fluoroguanosine
  modified_base 15: mod_base = um
  modified_base 16: mod_base = OTHER; note = 2'-fluorocytidine
  modified_base 17: mod_base = OTHER; note = 2'-O-methyladenosine
  modified_base 18: mod_base = gm
  modified_base 19: mod_base = um
  modified_base 20: mod_base = gm
  modified_base 21: mod_base = OTHER; note = 2'-O-methyluridine with a 3'-phosphorothioate linkage
  modified_base 22: mod_base = OTHER; note = 2'-O-methyladenosine with a 3'-phosphorothioate linkage
  modified_base 23: mod_base = cm
tggtgaagaccctgtcagtgtac

SEQ ID NO: 157 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = um
  modified_base 4: mod_base = OTHER; note = 2'-O-methyladenosine
  modified_base 5: mod_base = um
  modified_base 6: mod_base = OTHER; note = 2'-fluorocytidine
  modified_base 7: mod_base = um
  modified_base 8: mod_base = um
  modified_base 9: mod_base = um
  modified_base 10: mod_base = OTHER; note = 2'-O-methyladenosine
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluoroadenosine
  modified_base 15: mod_base = gm
  modified_base 16: mod_base = OTHER; note = 2'-fluoroguanosine
  modified_base 17: mod_base = cm
  modified_base 18: mod_base = um
  modified_base 19: mod_base = um
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tgtatctttatcaaggcttcaaa

SEQ ID NO: 158 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = cm
  modified_base 4: mod_base = um
  modified_base 5: mod_base = cm
  modified_base 6: mod_base = OTHER; note = 2'-fluorouridine
  modified_base 7: mod_base = OTHER; note = 2'-O-methyladenosine
  modified_base 8: mod_base = gm
  modified_base 9: mod_base = um
  modified_base 10: mod_base = gm
  modified_base 11: mod_base = OTHER; note = 2'-O-methyladenosine
  modified_base 12: mod_base = cm
  modified_base 13: mod_base = OTHER; note = 2'-O-methyladenosine
  modified_base 14: mod_base = OTHER; note = 2'-fluorocytidine
  modified_base 15: mod_base = um
  modified_base 16: mod_base = OTHER; note = 2'-fluorocytidine
  modified_base 17: mod_base = um
  modified_base 18: mod_base = cm
  modified_base 19: mod_base = OTHER; note = 2'-O-methyladenosine
  modified_base 20: mod_base = um
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
tcctctagtgacactctcatagg

SEQ ID NO: 159 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = OTHER; note = 2'-O-methyladenosine
  modified_base 4: mod_base = um
  modified_base 5: mod_base = gm
  modified_base 6: mod_base = OTHER; note = 2'-fluoroadenosine
  modified_base 7: mod_base = cm
  modified_base 8: mod_base = OTHER; note = 2'-O-methyladenosine
  modified_base 9: mod_base = gm
  modified_base 10: mod_base = um
  modified_base 11: mod_base = gm
  modified_base 12: mod_base = gm
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = um
taatgacagtggctgaaatgtct

SEQ ID NO: 160 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = cm
  modified_base 5: mod_base = um
  modified_base 6: mod_base = OTHER; note = 2'-fluorouridine
  modified_base 7: mod_base = gm
  modified_base 8: mod_base = um
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = OTHER; note = 2'-O-methyladenosine
  modified_base 18: mod_base = um
  modified_base 19: mod_base = cm
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = OTHER; note = 2'-O-methyladenosine
tgacttgttattcacaatcgaca

SEQ ID NO: 161 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = cm
  modified_base 4: mod_base = cm
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methyladenosine
  modified_base 9: mod_base = um
  modified_base 10: mod_base = OTHER; note = 2'-O-methyladenosine
  modified_base 11: mod_base = gm
  modified_base 12: mod_base = um
  modified_base 13: mod_base = gm
  modified_base 14: mod_base = OTHER; note = 2'-fluoroadenosine
  modified_base 15: mod_base = cm
  modified_base 16: mod_base = OTHER; note = 2'-fluorocytidine
  modified_base 17: mod_base = gm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyluridine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
taccaaaatagtgaccgtaataa

SEQ ID NO: 162 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = um
  modified_base 4: mod_base = cm
  modified_base 5: mod_base = um
  modified_base 6: mod_base = OTHER; note = 2'-O-methyladenosine
  modified_base 7: mod_base = OTHER; note = 2'-O-methyladenosine
  modified_base 8: mod_base = gm
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 163 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = um
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 164 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = OTHER; note = 2'-fluorouridine
  modified_base 4: mod_base = cm
  modified_base 5: mod_base = um
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 165 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = um
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluoroguanosine
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 166 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroadenosine with a 3'-phosphorothioate linkage
  modified_base 3: mod_base = OTHER; note = 2'-fluorouridine
  modified_base 4: mod_base = cm
  modified_base 5: mod_base = um
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluoroguanosine
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 167 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = gm
  modified_base 4: mod_base = um
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-fluoroadenosine
  modified_base 8: mod_base = cm
  modified_base 9: mod_base = um
  modified_base 10: mod_base = cm
  modified_base 11: mod_base = OTHER; note = 2'-O-methyladenosine
  modified_base 12: mod_base = um
  modified_base 13: mod_base = um
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
tcgtgaactcatttagacaaaat

SEQ ID NO: 168 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = OTHER; note = 2'-fluoroguanosine
  modified_base 4: mod_base = um
  modified_base 5: mod_base = gm
  modified_base 6: mod_base = OTHER; note = 2'-O-methyladenosine
  modified_base 7: mod_base = OTHER; note = 2'-fluoroadenosine
  modified_base 8: mod_base = cm
  modified_base 9: mod_base = um
  modified_base 10: mod_base = cm
  modified_base 11: mod_base = OTHER; note = 2'-O-methyladenosine
  modified_base 12: mod_base = um
  modified_base 13: mod_base = um
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
tcgtgaactcatttagacaaaat

SEQ ID NO: 169 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = gm
  modified_base 4: mod_base = um
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluorocytidine
  modified_base 9: mod_base = um
  modified_base 10: mod_base = cm
  modified_base 11: mod_base = OTHER; note = 2'-O-methyladenosine
  modified_base 12: mod_base = um
  modified_base 13: mod_base = um
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
tcgtgaactcatttagacaaaat

SEQ ID NO: 170 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-fluoroadenosine
  modified_base 6: mod_base = OTHER; note = 2'-O-methyladenosine
  modified_base 7: mod_base = OTHER; note = 2'-fluoroguanosine
  modified_base 8: mod_base = cm
  modified_base 9: mod_base = um
  modified_base 10: mod_base = gm
  modified_base 11: mod_base = gm
  modified_base 12: mod_base = um
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
ttcaaagctggtgtagttaaaat

SEQ ID NO: 171 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = OTHER; note = 2'-fluorocytidine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = cm
  modified_base 9: mod_base = um
  modified_base 10: mod_base = gm
  modified_base 11: mod_base = gm
  modified_base 12: mod_base = um
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
ttcaaagctggtgtagttaaaat

SEQ ID NO: 172 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-fluoroadenosine
  modified_base 6: mod_base = OTHER; note = 2'-O-methyladenosine
  modified_base 7: mod_base = gm
  modified_base 8: mod_base = OTHER; note = 2'-fluorocytidine
  modified_base 9: mod_base = um
  modified_base 10: mod_base = gm
  modified_base 11: mod_base = gm
  modified_base 12: mod_base = um
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
ttcaaagctggtgtagttaaaat

SEQ ID NO: 173 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-fluoroguanosine
  modified_base 6: mod_base = um
  modified_base 7: mod_base = OTHER; note = 2'-fluoroadenosine
  modified_base 8: mod_base = um
  modified_base 9: mod_base = um
  modified_base 10: mod_base = cm
  modified_base 11: mod_base = um
  modified_base 12: mod_base = gm
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
ttaagtattctgctgacaagaaa

SEQ ID NO: 174 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = OTHER; note = 2'-fluoroadenosine
  modified_base 4: mod_base = OTHER; note = 2'-O-methyladenosine
  modified_base 5: mod_base = gm
  modified_base 6: mod_base = um
  modified_base 7: mod_base = OTHER; note = 2'-fluoroadenosine
  modified_base 8: mod_base = um
  modified_base 9: mod_base = um
  modified_base 10: mod_base = cm
  modified_base 11: mod_base = um
  modified_base 12: mod_base = gm
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
ttaagtattctgctgacaagaaa

SEQ ID NO: 175 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-fluoroguanosine
  modified_base 6: mod_base = um
  modified_base 7: mod_base = OTHER; note = 2'-O-methyladenosine
  modified_base 8: mod_base = OTHER; note = 2'-fluorouridine
  modified_base 9: mod_base = um
  modified_base 10: mod_base = cm
  modified_base 11: mod_base = um
  modified_base 12: mod_base = gm
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
ttaagtattctgctgacaagaaa

SEQ ID NO: 176 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = cm
  modified_base 4: mod_base = um
  modified_base 5: mod_base = OTHER; note = 2'-fluoroadenosine
  modified_base 6: mod_base = um
  modified_base 7: mod_base = OTHER; note = 2'-fluorouridine
  modified_base 8: mod_base = gm
  modified_base 9: mod_base = gm
  modified_base 10: mod_base = um
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = um
tgctattggttcataggatgatt

SEQ ID NO: 177 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = OTHER; note = 2'-fluorocytidine
  modified_base 4: mod_base = um
  modified_base 5: mod_base = OTHER; note = 2'-O-methyladenosine
  modified_base 6: mod_base = um
  modified_base 7: mod_base = OTHER; note = 2'-fluorouridine
  modified_base 8: mod_base = gm
  modified_base 9: mod_base = gm
  modified_base 10: mod_base = um
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = um
tgctattggttcataggatgatt

SEQ ID NO: 178 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluoroguanosine with a 3'-phosphorothioate linkage
  modified_base 3: mod_base = cm
  modified_base 4: mod_base = um
  modified_base 5: mod_base = OTHER; note = 2'-fluoroadenosine
  modified_base 6: mod_base = um
  modified_base 7: mod_base = um
  modified_base 8: mod_base = OTHER; note = 2'-fluoroguanosine
  modified_base 9: mod_base = gm
  modified_base 10: mod_base = um
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = um
tgctattggttcataggatgatt

SEQ ID NO: 179 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = um
  modified_base 4: mod_base = um
  modified_base 5: mod_base = OTHER; note = 2'-fluorouridine
  modified_base 6: mod_base = OTHER; note = 2'-O-methyladenosine
  modified_base 7: mod_base = OTHER; note = 2'-fluorouridine
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = gm
  modified_base 13: mod_base = cm
  modified_base 14: mod_base = OTHER; note = 2'-fluorouridine
  modified_base 15: mod_base = um
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = gm
tctttatcaaggcttcaaaggtg

SEQ ID NO: 180 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = OTHER; note = 2'-fluorouridine
  modified_base 4: mod_base = um
  modified_base 5: mod_base = um
  modified_base 6: mod_base = OTHER; note = 2'-O-methyladenosine
  modified_base 7: mod_base = OTHER; note = 2'-fluorouridine
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = gm
  modified_base 13: mod_base = cm
  modified_base 14: mod_base = OTHER; note = 2'-fluorouridine
  modified_base 15: mod_base = um
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = gm
tctttatcaaggcttcaaaggtg

SEQ ID NO: 181 - RNA, 23 nt, synthetic construct
  source 1..23: mol_type = other RNA
  modified_base 1: mod_base = OTHER; note = 2'-O-methyluridine with a 3'-phosphorothioate linkage and a 5'-phosphate substitution
  modified_base 2: mod_base = OTHER; note = 2'-fluorocytidine with a 3'-phosphorothioate linkage
  modified_base 3: mod_base = um
  modified_base 4: mod_base = um
  modified_base 5: mod_base = OTHER; note = 2'-fluorouridine
  modified_base 6: mod_base = OTHER; note = 2'-O-methyladenosine
  modified_base 7: mod_base = um
  modified_base 8: mod_base = OTHER; note = 2'-fluorocytidine
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = gm
  modified_base 13: mod_base = cm
  modified_base 14: mod_base = OTHER; note = 2'-fluorouridine
  modified_base 15: mod_base = um
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = gm
tctttatcaaggcttcaaaggtg

SEQ ID NO: 182 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = cm
  modified_base 9: mod_base = um
  modified_base 10: mod_base = gm
  modified_base 11: mod_base = gm
  modified_base 12: mod_base = um
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
ttcaaagctggtgtagttaaaat

SEQ ID NO: 183 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = OTHER; note = 2'-fluorouridine
  modified_base 7: mod_base = OTHER; note = 2'-O-methyladenosine
  modified_base 8: mod_base = um
  modified_base 9: mod_base = um
  modified_base 10: mod_base = cm
  modified_base 11: mod_base = um
  modified_base 12: mod_base = gm
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
ttaagtattctgctgacaagaaa

SEQ ID NO: 184 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = um
  modified_base 4: mod_base = cm
  modified_base 5: mod_base = um
  modified_base 6: mod_base = OTHER; note = 2'-fluoroadenosine
  modified_base 7: mod_base = OTHER; note = 2'-O-methyladenosine
  modified_base 8: mod_base = gm
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 185 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = um
  modified_base 5: mod_base = um
  modified_base 6: mod_base = OTHER; note = 2'-fluoroadenosine
  modified_base 7: mod_base = gm
  modified_base 8: mod_base = cm
  modified_base 9: mod_base = gm
  modified_base 10: mod_base = OTHER; note = 2'-O-methyladenosine
  modified_base 11: mod_base = um
  modified_base 12: mod_base = gm
  modified_base 13: mod_base = OTHER; note = 2'-O-methyladenosine
  modified_base 14: mod_base = OTHER; note = 2'-fluoroadenosine
  modified_base 15: mod_base = gm
  modified_base 16: mod_base = OTHER; note = 2'-fluorouridine
  modified_base 17: mod_base = cm
  modified_base 18: mod_base = OTHER; note = 2'-O-methyladenosine
  modified_base 19: mod_base = cm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = gm
tgattagcgatgaagtcactgtg

SEQ ID NO: 186 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = cm
  modified_base 4: mod_base = gm
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = OTHER; note = 2'-O-methyladenosine
  modified_base 9: mod_base = OTHER; note = 2'-O-methyladenosine
  modified_base 10: mod_base = gm
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluorocytidine
  modified_base 15: mod_base = um
  modified_base 16: mod_base = OTHER; note = 2'-fluoroguanosine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = um
tgcgatgaagtcactgtgctcat

SEQ ID NO: 187 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = gm
  modified_base 4: mod_base = um
  modified_base 5: mod_base = gm
  modified_base 6: mod_base = OTHER; note = 2'-fluoroadenosine
  modified_base 7: mod_base = OTHER; note = 2'-O-methyladenosine
  modified_base 8: mod_base = cm
  modified_base 9: mod_base = um
  modified_base 10: mod_base = cm
  modified_base 11: mod_base = OTHER; note = 2'-O-methyladenosine
  modified_base 12: mod_base = um
  modified_base 13: mod_base = um
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
tcgtgaactcatttagacaaaat

SEQ ID NO: 188 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = um
  modified_base 4: mod_base = um
  modified_base 5: mod_base = um
  modified_base 6: mod_base = OTHER; note = 2'-fluoroadenosine
  modified_base 7: mod_base = um
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = gm
  modified_base 13: mod_base = cm
  modified_base 14: mod_base = OTHER; note = 2'-fluorouridine
  modified_base 15: mod_base = um
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = gm
tctttatcaaggcttcaaaggtg

SEQ ID NO: 189 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = cm
  modified_base 4: mod_base = um
  modified_base 5: mod_base = OTHER; note = 2'-O-methyladenosine
  modified_base 6: mod_base = OTHER; note = 2'-fluorouridine
  modified_base 7: mod_base = um
  modified_base 8: mod_base = gm
  modified_base 9: mod_base = gm
  modified_base 10: mod_base = um
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = um
tgctattggttcataggatgatt

SEQ ID NO: 190 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = gm
  modified_base 4: mod_base = um
  modified_base 5: mod_base = cm
  modified_base 6: mod_base = OTHER; note = 2'-fluoroadenosine
  modified_base 7: mod_base = gm
  modified_base 8: mod_base = cm
  modified_base 9: mod_base = OTHER; note = 2'-O-methyladenosine
  modified_base 10: mod_base = cm
  modified_base 11: mod_base = um
  modified_base 12: mod_base = gm
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = gm
  modified_base 19: mod_base = um
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
ttgtcagcactgaaagagttcaa

SEQ ID NO: 191 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = cm
  modified_base 4: mod_base = cm
  modified_base 5: mod_base = cm
  modified_base 6: mod_base = OTHER; note = 2'-fluorocytidine
  modified_base 7: mod_base = OTHER; note = 2'-O-methyladenosine
  modified_base 8: mod_base = OTHER; note = 2'-O-methyladenosine
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-O-methyladenosine
  modified_base 13: mod_base = gm
  modified_base 14: mod_base = OTHER; note = 2'-fluoroadenosine
  modified_base 15: mod_base = gm
  modified_base 16: mod_base = OTHER; note = 2'-fluoroadenosine
  modified_base 17: mod_base = um
  modified_base 18: mod_base = um
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = gm
taccccaacaaagagattcagtg

SEQ ID NO: 192 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = gm
  modified_base 4: mod_base = OTHER; note = 2'-O-methyladenosine
  modified_base 5: mod_base = gm
  modified_base 6: mod_base = OTHER; note = 2'-fluorocytidine
  modified_base 7: mod_base = um
  modified_base 8: mod_base = OTHER; note = 2'-O-methyladenosine
  modified_base 9: mod_base = gm
  modified_base 10: mod_base = OTHER; note = 2'-O-methyladenosine
  modified_base 11: mod_base = um
  modified_base 12: mod_base = gm
  modified_base 13: mod_base = um
  modified_base 14: mod_base = OTHER; note = 2'-fluorocytidine
  modified_base 15: mod_base = cm
  modified_base 16: mod_base = OTHER; note = 2'-fluorouridine
  modified_base 17: mod_base = cm
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methyluridine with a 3'-phosphorothioate linkage
  modified_base 22: mod_base = OTHER; note = 2'-O-methyluridine with a 3'-phosphorothioate linkage
  modified_base 23: mod_base = gm
ttgagctagatgtcctcatgttg

SEQ ID NO: 193 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = um
  modified_base 9: mod_base = OTHER; note = 2'-O-methyladenosine
  modified_base 10: mod_base = gm
  modified_base 11: mod_base = um
  modified_base 12: mod_base = gm
  modified_base 13: mod_base = OTHER; note = 2'-O-methyladenosine
  modified_base 14: mod_base = OTHER; note = 2'-fluorocytidine
  modified_base 15: mod_base = cm
  modified_base 16: mod_base = OTHER; note = 2'-fluoroguanosine
  modified_base 17: mod_base = um
  modified_base 18: mod_base = OTHER; note = 2'-O-methyladenosine
  modified_base 19: mod_base = OTHER; note = 2'-O-methyladenosine
  modified_base 20: mod_base = um
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tccaaaatagtgaccgtaataaa

SEQ ID NO: 194 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = OTHER; note = 2'-fluorocytidine
  modified_base 7: mod_base = cm
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = gm
  modified_base 13: mod_base = OTHER; note = 2'-O-methyladenosine
  modified_base 14: mod_base = OTHER; note = 2'-fluoroguanosine
  modified_base 15: mod_base = OTHER; note = 2'-O-methyladenosine
  modified_base 16: mod_base = OTHER; note = 2'-fluoroadenosine
  modified_base 17: mod_base = um
  modified_base 18: mod_base = um
  modified_base 19: mod_base = gm
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
tcaagcccaaagagaattgtctt

SEQ ID NO: 195 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = cm
  modified_base 4: mod_base = gm
  modified_base 5: mod_base = OTHER; note = 2'-O-methyladenosine
  modified_base 6: mod_base = OTHER; note = 2'-fluoroadenosine
  modified_base 7: mod_base = gm
  modified_base 8: mod_base = gm
  modified_base 9: mod_base = um
  modified_base 10: mod_base = OTHER; note = 2'-O-methyladenosine
  modified_base 11: mod_base = gm
  modified_base 12: mod_base = gm
  modified_base 13: mod_base = gm
  modified_base 14: mod_base = OTHER; note = 2'-fluorocytidine
  modified_base 15: mod_base = um
  modified_base 16: mod_base = OTHER; note = 2'-fluorocytidine
  modified_base 17: mod_base = OTHER; note = 2'-O-methyladenosine
  modified_base 18: mod_base = um
  modified_base 19: mod_base = gm
  modified_base 20: mod_base = cm
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = um
ttcgaaggtagggctcatgccat

SEQ ID NO: 196 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = cm
  modified_base 5: mod_base = um
  modified_base 6: mod_base = OTHER; note = 2'-fluoroguanosine
  modified_base 7: mod_base = OTHER; note = 2'-O-methyladenosine
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methylcytidine with a 3'-phosphorothioate linkage
  modified_base 22: mod_base = OTHER; note = 2'-O-methylcytidine with a 3'-phosphorothioate linkage
  modified_base 23: mod_base = um
ttactgacaacactcattgccct

SEQ ID NO: 197 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = um
  modified_base 4: mod_base = OTHER; note = 2'-O-methyladenosine
  modified_base 5: mod_base = gm
  modified_base 6: mod_base = OTHER; note = 2'-fluoroguanosine
  modified_base 7: mod_base = OTHER; note = 2'-O-methyladenosine
  modified_base 8: mod_base = gm
  modified_base 9: mod_base = cm
  modified_base 10: mod_base = um
  modified_base 11: mod_base = gm
  modified_base 12: mod_base = cm
  modified_base 13: mod_base = um
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = cm
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = gm
tctaggagctgcttgccaaccag

SEQ ID NO: 198 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = um
  modified_base 4: mod_base = OTHER; note = 2'-O-methyladenosine
  modified_base 5: mod_base = gm
  modified_base 6: mod_base = OTHER; note = 2'-fluoroguanosine
  modified_base 7: mod_base = cm
  modified_base 8: mod_base = OTHER; note = 2'-O-methyladenosine
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = gm
  modified_base 13: mod_base = OTHER; note = 2'-O-methyladenosine
  modified_base 14: mod_base = OTHER; note = 2'-fluorouridine
  modified_base 15: mod_base = gm
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = um
tttaggcagaagatggtgaggat

SEQ ID NO: 199 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyladenosine
  modified_base 8: mod_base = cm
  modified_base 9: mod_base = um
  modified_base 10: mod_base = cm
  modified_base 11: mod_base = cm
  modified_base 12: mod_base = OTHER; note = 2'-O-methyladenosine
  modified_base 13: mod_base = um
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluorouridine
  modified_base 17: mod_base = cm
  modified_base 18: mod_base = um
  modified_base 19: mod_base = gm
  modified_base 20: mod_base = um
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = cm
tgaaagactccatcatctgtgac

SEQ ID NO: 200 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = cm
  modified_base 4: mod_base = um
  modified_base 5: mod_base = gm
  modified_base 6: mod_base = OTHER; note = 2'-fluorouridine
  modified_base 7: mod_base = gm
  modified_base 8: mod_base = cm
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = cm
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = gm
  modified_base 18: mod_base = cm
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = gm
tgctgtgccaacatatgccaggg

SEQ ID NO: 201 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = um
  modified_base 5: mod_base = gm
  modified_base 6: mod_base = OTHER; note = 2'-fluoroadenosine
  modified_base 7: mod_base = um
  modified_base 8: mod_base = OTHER; note = 2'-O-methyladenosine
  modified_base 9: mod_base = cm
  modified_base 10: mod_base = um
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluorocytidine
  modified_base 17: mod_base = OTHER; note = 2'-O-methyladenosine
  modified_base 18: mod_base = cm
  modified_base 19: mod_base = um
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = um
ttatgatactgacaacactcatt

SEQ ID NO: 202 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = um
  modified_base 4: mod_base = um
  modified_base 5: mod_base = gm
  modified_base 6: mod_base = OTHER; note = 2'-fluorocytidine
  modified_base 7: mod_base = um
  modified_base 8: mod_base = OTHER; note = 2'-O-methyladenosine
  modified_base 9: mod_base = um
  modified_base 10: mod_base = um
  modified_base 11: mod_base = gm
  modified_base 12: mod_base = gm
  modified_base 13: mod_base = um
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = OTHER; note = 2'-O-methyladenosine
  modified_base 19: mod_base = gm
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = gm
tgttgctattggttcataggatg

SEQ ID NO: 203 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = um
  modified_base 4: mod_base = um
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = OTHER; note = 2'-O-methyladenosine
  modified_base 9: mod_base = um
  modified_base 10: mod_base = OTHER; note = 2'-O-methyladenosine
  modified_base 11: mod_base = cm
  modified_base 12: mod_base = um
  modified_base 13: mod_base = gm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = OTHER; note = 2'-O-methyladenosine
tgttatgatactgacaacactca

SEQ ID NO: 204 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-fluoroguanosine
  modified_base 7: mod_base = um
  modified_base 8: mod_base = cm
  modified_base 9: mod_base = OTHER; note = 2'-O-methyladenosine
  modified_base 10: mod_base = gm
  modified_base 11: mod_base = OTHER; note = 2'-O-methyladenosine
  modified_base 12: mod_base = cm
  modified_base 13: mod_base = um
  modified_base 14: mod_base = OTHER; note = 2'-fluorouridine
  modified_base 15: mod_base = gm
  modified_base 16: mod_base = OTHER; note = 2'-fluorouridine
  modified_base 17: mod_base = um
  modified_base 18: mod_base = OTHER; note = 2'-O-methyladenosine
  modified_base 19: mod_base = um
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyladenosine with a 3'-phosphorothioate linkage
  modified_base 23: mod_base = cm
ttgcagtcagacttgttattcac

SEQ ID NO: 205 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = um
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = cm
  modified_base 9: mod_base = cm
  modified_base 10: mod_base = OTHER; note = 2'-O-methyladenosine
  modified_base 11: mod_base = gm
  modified_base 12: mod_base = gm
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroadenosine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methylguanosine with a 3'-phosphorothioate linkage
  modified_base 22: mod_base = OTHER; note = 2'-O-methylcytidine with a 3'-phosphorothioate linkage
  modified_base 23: mod_base = um
tcatatgccagggtaatgacgct

SEQ ID NO: 206 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = gm
  modified_base 4: mod_base = um
  modified_base 5: mod_base = gm
  modified_base 6: mod_base = OTHER; note = 2'-fluoroadenosine
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = cm
  modified_base 11: mod_base = cm
  modified_base 12: mod_base = cm
  modified_base 13: mod_base = um
  modified_base 14: mod_base = OTHER; note = 2'-fluoroguanosine
  modified_base 15: mod_base = um
  modified_base 16: mod_base = OTHER; note = 2'-fluorocytidine
  modified_base 17: mod_base = OTHER; note = 2'-O-methyladenosine
  modified_base 18: mod_base = gm
  modified_base 19: mod_base = um
  modified_base 20: mod_base = gm
  modified_base 21: mod_base = OTHER; note = 2'-O-methyluridine with a 3'-phosphorothioate linkage
  modified_base 22: mod_base = OTHER; note = 2'-O-methyladenosine with a 3'-phosphorothioate linkage
  modified_base 23: mod_base = cm
tggtgaagaccctgtcagtgtac

SEQ ID NO: 207 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = um
  modified_base 4: mod_base = OTHER; note = 2'-O-methyladenosine
  modified_base 5: mod_base = um
  modified_base 6: mod_base = OTHER; note = 2'-fluorocytidine
  modified_base 7: mod_base = um
  modified_base 8: mod_base = um
  modified_base 9: mod_base = um
  modified_base 10: mod_base = OTHER; note = 2'-O-methyladenosine
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluoroadenosine
  modified_base 15: mod_base = gm
  modified_base 16: mod_base = OTHER; note = 2'-fluoroguanosine
  modified_base 17: mod_base = cm
  modified_base 18: mod_base = um
  modified_base 19: mod_base = um
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
tgtatctttatcaaggcttcaaa

SEQ ID NO: 208 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = cm
  modified_base 4: mod_base = um
  modified_base 5: mod_base = cm
  modified_base 6: mod_base = OTHER; note = 2'-fluorouridine
  modified_base 7: mod_base = OTHER; note = 2'-O-methyladenosine
  modified_base 8: mod_base = gm
  modified_base 9: mod_base = um
  modified_base 10: mod_base = gm
  modified_base 11: mod_base = OTHER; note = 2'-O-methyladenosine
  modified_base 12: mod_base = cm
  modified_base 13: mod_base = OTHER; note = 2'-O-methyladenosine
  modified_base 14: mod_base = OTHER; note = 2'-fluorocytidine
  modified_base 15: mod_base = um
  modified_base 16: mod_base = OTHER; note = 2'-fluorocytidine
  modified_base 17: mod_base = um
  modified_base 18: mod_base = cm
  modified_base 19: mod_base = OTHER; note = 2'-O-methyladenosine
  modified_base 20: mod_base = um
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
tcctctagtgacactctcatagg

SEQ ID NO: 209 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = OTHER; note = 2'-O-methyladenosine
  modified_base 4: mod_base = um
  modified_base 5: mod_base = gm
  modified_base 6: mod_base = OTHER; note = 2'-fluoroadenosine
  modified_base 7: mod_base = cm
  modified_base 8: mod_base = OTHER; note = 2'-O-methyladenosine
  modified_base 9: mod_base = gm
  modified_base 10: mod_base = um
  modified_base 11: mod_base = gm
  modified_base 12: mod_base = gm
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methyluridine with a 3'-phosphorothioate linkage
  modified_base 22: mod_base = OTHER; note = 2'-O-methylcytidine with a 3'-phosphorothioate linkage
  modified_base 23: mod_base = um
taatgacagtggctgaaatgtct

SEQ ID NO: 210 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = OTHER; note = 2'-O-methyladenosine
  modified_base 4: mod_base = cm
  modified_base 5: mod_base = um
  modified_base 6: mod_base = OTHER; note = 2'-fluorouridine
  modified_base 7: mod_base = gm
  modified_base 8: mod_base = um
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = OTHER; note = 2'-O-methyladenosine
  modified_base 18: mod_base = um
  modified_base 19: mod_base = cm
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = OTHER; note = 2'-O-methyladenosine
tgacttgttattcacaatcgaca

SEQ ID NO: 211 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = cm
  modified_base 4: mod_base = cm
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methyladenosine
  modified_base 9: mod_base = um
  modified_base 10: mod_base = OTHER; note = 2'-O-methyladenosine
  modified_base 11: mod_base = gm
  modified_base 12: mod_base = um
  modified_base 13: mod_base = gm
  modified_base 14: mod_base = OTHER; note = 2'-fluoroadenosine
  modified_base 15: mod_base = cm
  modified_base 16: mod_base = OTHER; note = 2'-fluorocytidine
  modified_base 17: mod_base = gm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyluridine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
taccaaaatagtgaccgtaataa

SEQ ID NO: 212 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = um
  modified_base 4: mod_base = cm
  modified_base 5: mod_base = um
  modified_base 6: mod_base = OTHER; note = 2'-O-methyladenosine
  modified_base 7: mod_base = OTHER; note = 2'-O-methyladenosine
  modified_base 8: mod_base = gm
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 213 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = um
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 214 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = OTHER; note = 2'-fluorouridine
  modified_base 4: mod_base = cm
  modified_base 5: mod_base = um
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 215 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = um
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluoroguanosine
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 216 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = OTHER; note = 2'-fluorouridine
  modified_base 4: mod_base = cm
  modified_base 5: mod_base = um
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluoroguanosine
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 217 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = gm
  modified_base 4: mod_base = um
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-fluoroadenosine
  modified_base 8: mod_base = cm
  modified_base 9: mod_base = um
  modified_base 10: mod_base = cm
  modified_base 11: mod_base = OTHER; note = 2'-O-methyladenosine
  modified_base 12: mod_base = um
  modified_base 13: mod_base = um
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
tcgtgaactcatttagacaaaat

SEQ ID NO: 218 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-fluoroguanosine
  modified_base 4: mod_base = um
  modified_base 5: mod_base = gm
  modified_base 6: mod_base = OTHER; note = 2'-O-methyladenosine
  modified_base 7: mod_base = OTHER; note = 2'-fluoroadenosine
  modified_base 8: mod_base = cm
  modified_base 9: mod_base = um
  modified_base 10: mod_base = cm
  modified_base 11: mod_base = OTHER; note = 2'-O-methyladenosine
  modified_base 12: mod_base = um
  modified_base 13: mod_base = um
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
tcgtgaactcatttagacaaaat

SEQ ID NO: 219 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = gm
  modified_base 4: mod_base = um
  modified_base 5: mod_base = OTHER; note = 2'-fluoroguanosine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluorocytidine
  modified_base 9: mod_base = um
  modified_base 10: mod_base = cm
  modified_base 11: mod_base = OTHER; note = 2'-O-methyladenosine
  modified_base 12: mod_base = um
  modified_base 13: mod_base = um
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = cm
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
tcgtgaactcatttagacaaaat

SEQ ID NO: 220 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-fluoroadenosine
  modified_base 6: mod_base = OTHER; note = 2'-O-methyladenosine
  modified_base 7: mod_base = OTHER; note = 2'-fluoroguanosine
  modified_base 8: mod_base = cm
  modified_base 9: mod_base = um
  modified_base 10: mod_base = gm
  modified_base 11: mod_base = gm
  modified_base 12: mod_base = um
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
ttcaaagctggtgtagttaaaat

SEQ ID NO: 221 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = OTHER; note = 2'-fluorocytidine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = cm
  modified_base 9: mod_base = um
  modified_base 10: mod_base = gm
  modified_base 11: mod_base = gm
  modified_base 12: mod_base = um
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
ttcaaagctggtgtagttaaaat

SEQ ID NO: 222 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-fluoroadenosine
  modified_base 6: mod_base = OTHER; note = 2'-O-methyladenosine
  modified_base 7: mod_base = gm
  modified_base 8: mod_base = OTHER; note = 2'-fluorocytidine
  modified_base 9: mod_base = um
  modified_base 10: mod_base = gm
  modified_base 11: mod_base = gm
  modified_base 12: mod_base = um
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = um
ttcaaagctggtgtagttaaaat

SEQ ID NO: 223 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-fluoroguanosine
  modified_base 6: mod_base = um
  modified_base 7: mod_base = OTHER; note = 2'-fluoroadenosine
  modified_base 8: mod_base = um
  modified_base 9: mod_base = um
  modified_base 10: mod_base = cm
  modified_base 11: mod_base = um
  modified_base 12: mod_base = gm
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
ttaagtattctgctgacaagaaa

SEQ ID NO: 224 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = OTHER; note = 2'-fluoroadenosine
  modified_base 4: mod_base = OTHER; note = 2'-O-methyladenosine
  modified_base 5: mod_base = gm
  modified_base 6: mod_base = um
  modified_base 7: mod_base = OTHER; note = 2'-fluoroadenosine
  modified_base 8: mod_base = um
  modified_base 9: mod_base = um
  modified_base 10: mod_base = cm
  modified_base 11: mod_base = um
  modified_base 12: mod_base = gm
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
ttaagtattctgctgacaagaaa

SEQ ID NO: 225 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-fluoroguanosine
  modified_base 6: mod_base = um
  modified_base 7: mod_base = OTHER; note = 2'-O-methyladenosine
  modified_base 8: mod_base = OTHER; note = 2'-fluorouridine
  modified_base 9: mod_base = um
  modified_base 10: mod_base = cm
  modified_base 11: mod_base = um
  modified_base 12: mod_base = gm
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = cm
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = OTHER; note = 2'-O-methyladenosine
ttaagtattctgctgacaagaaa

SEQ ID NO: 226 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = cm
  modified_base 4: mod_base = um
  modified_base 5: mod_base = OTHER; note = 2'-fluoroadenosine
  modified_base 6: mod_base = um
  modified_base 7: mod_base = OTHER; note = 2'-fluorouridine
  modified_base 8: mod_base = gm
  modified_base 9: mod_base = gm
  modified_base 10: mod_base = um
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = um
tgctattggttcataggatgatt

SEQ ID NO: 227 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = OTHER; note = 2'-fluorocytidine
  modified_base 4: mod_base = um
  modified_base 5: mod_base = OTHER; note = 2'-O-methyladenosine
  modified_base 6: mod_base = um
  modified_base 7: mod_base = OTHER; note = 2'-fluorouridine
  modified_base 8: mod_base = gm
  modified_base 9: mod_base = gm
  modified_base 10: mod_base = um
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = um
tgctattggttcataggatgatt

SEQ ID NO: 228 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = cm
  modified_base 4: mod_base = um
  modified_base 5: mod_base = OTHER; note = 2'-fluoroadenosine
  modified_base 6: mod_base = um
  modified_base 7: mod_base = um
  modified_base 8: mod_base = OTHER; note = 2'-fluoroguanosine
  modified_base 9: mod_base = gm
  modified_base 10: mod_base = um
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = OTHER; note = 2'-O-methyladenosine
  modified_base 19: mod_base = um
  modified_base 20: mod_base = gm
  modified_base 21: mod_base = OTHER; note = 2'-O-methyladenosine with a 3'-phosphorothioate linkage
  modified_base 22: mod_base = OTHER; note = 2'-O-methyluridine with a 3'-phosphorothioate linkage
  modified_base 23: mod_base = um
tgctattggttcataggatgatt

SEQ ID NO: 229 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = um
  modified_base 4: mod_base = um
  modified_base 5: mod_base = OTHER; note = 2'-fluorouridine
  modified_base 6: mod_base = OTHER; note = 2'-O-methyladenosine
  modified_base 7: mod_base = OTHER; note = 2'-fluorouridine
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = gm
  modified_base 13: mod_base = cm
  modified_base 14: mod_base = OTHER; note = 2'-fluorouridine
  modified_base 15: mod_base = um
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = gm
tctttatcaaggcttcaaaggtg

SEQ ID NO: 230 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-fluorouridine
  modified_base 4: mod_base = um
  modified_base 5: mod_base = um
  modified_base 6: mod_base = OTHER; note = 2'-O-methyladenosine
  modified_base 7: mod_base = OTHER; note = 2'-fluorouridine
  modified_base 8: mod_base = cm
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = gm
  modified_base 13: mod_base = cm
  modified_base 14: mod_base = OTHER; note = 2'-fluorouridine
  modified_base 15: mod_base = um
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = gm
tctttatcaaggcttcaaaggtg

SEQ ID NO: 231 - RNA, 23 nt, synthetic construct
  source 1..23: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = um
  modified_base 4: mod_base = um
  modified_base 5: mod_base = OTHER; note = 2'-fluorouridine
  modified_base 6: mod_base = OTHER; note = 2'-O-methyladenosine
  modified_base 7: mod_base = um
  modified_base 8: mod_base = OTHER; note = 2'-fluorocytidine
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = gm
  modified_base 13: mod_base = cm
  modified_base 14: mod_base = OTHER; note = 2'-fluorouridine
  modified_base 15: mod_base = um
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyluridine with a 3'-phosphorothioate linkage
  modified_base 23: mod_base = gm
tctttatcaaggcttcaaaggtg

SEQ ID NO: 232 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = gm
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = OTHER; note = 2'-O-C16H33 adenosine
  modified_base 14: mod_base = cm
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
tgtcagcagaatacttagata

SEQ ID NO: 233 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphate substitution
  modified_base 2: mod_base = OTHER; note = 2'-fluoroadenosine with a 3'-phosphorothioate linkage
  modified_base 3: mod_base = um
  modified_base 4: mod_base = cm
  modified_base 5: mod_base = um
  modified_base 6: mod_base = OTHER; note = 2'-fluoroadenosine
  modified_base 7: mod_base = OTHER; note = 2'-O-methyladenosine
  modified_base 8: mod_base = gm
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 234 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-C3H6-NH(CO)-C2H4-SS-C12H25 uridine
  modified_base 13: mod_base = OTHER; note = 2'-O-methyladenosine
  modified_base 14: mod_base = cm
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
tgtcagcagaatacttagata

SEQ ID NO: 235 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphate substitution
  modified_base 2: mod_base = OTHER; note = 2'-fluoroadenosine with a 3'-phosphorothioate linkage
  modified_base 3: mod_base = um
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluoroguanosine
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 236 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = gm
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-C16H33 uridine
  modified_base 13: mod_base = OTHER; note = 2'-O-methyladenosine
  modified_base 14: mod_base = cm
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
tgtcagcagaatacttagata

SEQ ID NO: 237 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = gm
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = OTHER; note = 2'-O-C16H33 uridine
  modified_base 14: mod_base = cm
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
tgtcagcagaattcttagata

SEQ ID NO: 238 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-C16H33 uridine
  modified_base 13: mod_base = OTHER; note = 2'-O-methyladenosine
  modified_base 14: mod_base = cm
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
tgtcagcagaatacttagata

SEQ ID NO: 239 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphate substitution
  modified_base 2: mod_base = OTHER; note = 2'-fluoroadenosine with a 3'-phosphorothioate linkage
  modified_base 3: mod_base = um
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag

SEQ ID NO: 240 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = OTHER; note = 2'-O-C16H33 adenosine
  modified_base 14: mod_base = cm
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
tgtcagcagaatacttagata

SEQ ID NO: 241 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = gm
  modified_base 7: mod_base = cm
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = OTHER; note = 2'-O-C3H6-NH(CO)-C2H4-SS-C12H25 uridine
  modified_base 14: mod_base = cm
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
tgtcagcagaattcttagata

SEQ ID NO: 242 - RNA, 23 nt, synthetic construct
  source 1..23: mol_type = other RNA
  modified_base 1: mod_base = OTHER; note = 2'-O-methyluridine with a 3'-phosphorothioate linkage and a 5'-vinylphosphate substitution
  modified_base 2: mod_base = OTHER; note = 2'-fluoroadenosine with a 3'-phosphorothioate linkage
  modified_base 3: mod_base = um
  modified_base 4: mod_base = cm
  modified_base 5: mod_base = um
  modified_base 6: mod_base = OTHER; note = 2'-O-methyladenosine
  modified_base 7: mod_base = OTHER; note = 2'-O-methyladenosine
  modified_base 8: mod_base = gm
  modified_base 9: mod_base = um
  modified_base 10: mod_base = OTHER; note = 2'-O-methyladenosine
  modified_base 11: mod_base = um
  modified_base 12: mod_base = um
  modified_base 13: mod_base = cm
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = um
  modified_base 18: mod_base = gm
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methyladenosine with a 3'-phosphorothioate linkage
  modified_base 23: mod_base = gm
tatctaagtattctgctgacaag